(19) United States
(12) Patent Application Publication
(10) Pub. No.: US 2023/0102520 A1
(43) Pub. Date: Mar. 30, 2023

(54) INHIBITORS OF LOW MOLECULAR WEIGHT PROTEIN TYROSINE PHOSPHATASE (LMPTP) AND USES THEREOF

(71) Applicant: Sanford Burnham Prebys Medical Discovery Institute, La Jolla, CA (US)

(72) Inventors: Anthony B. PINKERTON, Rancho Santa Fe, CA (US); Robert J. ARDECKY, Encinitas, CA (US); Jiwen ZOU, San Diego, CA (US)

(21) Appl. No.: 17/624,052

(22) PCT Filed: Jul. 2, 2020
(86) PCT No.: PCT/US2020/040710

§ 371 Date: Dec. 30, 2021

Related U.S. Application Data
(60) Provisional application No. 62/870,207, filed on Jul. 3, 2019.

(51) Int. Cl. C07D 473/34; C07D 519/00; A61K 9/08; A61K 9/20; A61K 9/48; A61K 9/06; A61K 9/00

(52) U.S. Cl. CPC C07D 473/34; C07D 519/00; A61K 9/08; A61K 9/2054; A61K 9/4825; A61K 9/06; A61K 9/0053; A61K 9/0014

(57) ABSTRACT

Protein tyrosine phosphatases (PTPs) are key regulators of metabolism and insulin signaling. As a negative regulator of insulin signaling, the low molecular weight protein tyrosine phosphatase (LMPTP) is a target for insulin resistance and related conditions. Described herein are compounds capable of modulating the level of activity of LMPTP, compositions, and methods of using these compounds and compositions.

### CROSS REFERENCE

This application claims the benefit of U.S. Provisional Application No. 62/870,207, filed Jul. 3, 2019, which is incorporated by reference in its entirety.

### STATEMENT AS TO FEDERALLY SPONSORED RESEARCH

This invention was made with the support of the United States government under Research Project Grant R01 DK106233 awarded by the National Institutes of Health. The government has certain rights in the invention

### FIELD OF THE INVENTION

Described herein are inhibitors of low molecular weight protein tyrosine phosphatase (LMPTP), methods of making such compounds, pharmaceutical compositions and medicaments comprising such compounds, and methods of using such compounds in the treatment of conditions, diseases, or disorders associated with LMPTP activity.

### BACKGROUND OF THE INVENTION

Obesity is frequently complicated by a combination of metabolic and cardiovascular anomalies, called the metabolic syndrome, which significantly increases morbidity and mortality of affected individuals. Insulin resistance is an important component of the metabolic syndrome. Protein tyrosine phosphatases (PTPs), including low molecular weight protein tyrosine phosphatase (LMPTP) regulate insulin signaling. LMPTP is highly expressed in liver, muscle, adipocytes, heart and other tissues. Genetic association studies in humans support a negative role for LMPTP in insulin resistance and the metabolic complications of obesity.

### BRIEF SUMMARY OF THE INVENTION

Described herein are compounds capable of modulating the level of activity of low molecular weight protein tyrosine phosphatase (LMPTP) and compositions, and methods of using these compounds and compositions.

In one aspect, described herein is a compound that has the structure of Formula (I), or a pharmaceutically acceptable salt, or solvate thereof:

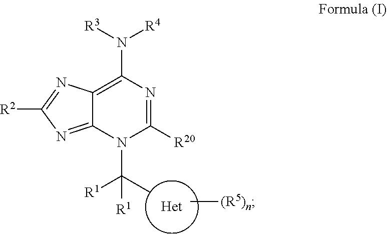

wherein:
Ring Het is heteroaryl;
each R¹ is independently hydrogen, —F, or —CH₃;
R² is halogen, —CN, —OH, —OR^{a}, —SH, —SR^{a}, C₁-C₆ alkyl, C₂-C₆ alkenyl, C₂-C₆ alkynyl, or C₁-C₆ heteroalkyl; wherein alkyl, alkenyl, alkynyl and C₁-C₆ heteroalkyl is unsubstituted or substituted with one, two, or three R⁶;
each R⁶ is independently halogen, —CN, —OH, —OR^{a}, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —NR^{b}C(═O)R^{a};
or R² is

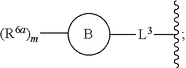

L³ is absent or C₁-C₆ alkylene;
Ring B is phenyl, heteroaryl, cycloalkyl, or heterocycloalkyl;
each R^{6a} is independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, C₁-C₆ heteroalkyl, or cycloalkyl which is unsubstituted or substituted with one, two, or three halogen, C₁-C₆ alkyl, or C₁-C₆ haloalkyl;
or two R^{6a} groups join together with the intervening atoms of ring B that connect the two R^{6a} groups to form a ring that is unsubstituted or substituted with one, two, or three halogen, —CN, —OH, C₁-C₆ alkyl, C₁-C₆ alkoxy, C₁-C₆ haloalkyl or C₁-C₆ haloalkoxy;
m is 0, 1, 2, 3, 4, or 5;
R³ is hydrogen, C₁-C₆ alkyl, C₂-C₆ alkenyl, C₂-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are independently unsubstituted or substituted with one, two, or three R¹⁰;
R⁴ is hydrogen, C₁-C₆ alkyl, C₂-C₆ alkenyl, C₂-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are independently unsubstituted or substituted with one, two, or three R¹¹;
or R⁴ is -L¹-L²-R⁷;
   L¹ is —C(═O)—, —S(═O)—, —S(═O)₂—, or C₁-C₄ alkylene;
   L² is absent or —CH₂—;
   R⁷ is C₁-C₆ alkyl, C₂-C₆ alkenyl, C₂-C₆ alkynyl, cycloalkyl, heterocycloalkyl, aryl, monocyclic heteroaryl that contains 1-4 N atoms and 0-2 O or S atoms, monocyclic heteroaryl that contains 0-4 N atoms and 1 S atom, or bicyclic heteroaryl that contains 0-4 N atoms and 0-2 O or S atoms; wherein each alkyl, alkenyl, alkynyl, cycloalkyl, heterocycloalkyl, aryl, and heteroaryl is unsubstituted or substituted with one, two, or three R⁸;
   each R⁸ is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, C₁-C₆ heteroalkyl, or cycloalkyl which is unsubstituted or substituted with one, two, or three halogen, C₁-C₆ alkyl, or C₁-C₆ haloalkyl;
or R³ and R⁴ are taken together with the nitrogen atom to which they are attached to form a heterocycloalkyl which is unsubstituted or substituted with one, two, or three R¹²;
each R⁵ is independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, C₁-C₆ heteroalkyl, or cycloalkyl which is unsubstituted or substituted with one, two, or three halogen, C₁-C₆ alkyl, or C₁-C₆ haloalkyl;
n is 0, 1, 2, 3, 4, or 5;
each R¹⁰, R¹¹, and R¹² is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(O)R^{a}, —NR^{b}C(═O)OR^{b}, C₁-C₆ alkyl, C₁-C₆ haloalkyl, C₁-C₆ hydroxyalkyl, C₁-C₆ heteroalkyl, or cycloalkyl which is unsubstituted or substituted with one, two, or three halogen, C₁-C₆ alkyl, or C₁-C₆ haloalkyl;
R²⁰ is hydrogen, halogen, —CN, —OH, —OR^{a}, —SH, —SR^{a}, C₁-C₆ alkyl, C₁-C₆ haloalkyl, C₁-C₆ hydroxyalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl; and
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl;
or two R^{b} groups on a nitrogen atom are taken together with the nitrogen atom to which they are attached to form a heterocycloalkyl which is unsubstituted or substituted with one, two, or three halogen, C₁-C₆ alkyl, or C₁-C₆ haloalkyl;
provided that the compound is not 3-[(3,5-dichloro(4-pyridyl))methyl]-8-phenyl-3-hydropurine-6-ylamine.

Any combination of the groups described above or below for the various variables is contemplated herein. Throughout the specification, groups and substituents thereof are chosen by one skilled in the field to provide stable moieties and compounds.

In one aspect, provided herein is a pharmaceutical composition comprising a compound disclosed herein, or a pharmaceutically acceptable salt, or solvate thereof, and at least one pharmaceutically acceptable excipient.

In some embodiments, the compound disclosed herein, or a pharmaceutically acceptable salt thereof, is formulated for administration to a mammal by intravenous administration, subcutaneous administration, oral administration, inhalation, nasal administration, dermal administration, or ophthalmic administration. In some embodiments, the compound disclosed herein, or a pharmaceutically acceptable salt thereof, is in the form of a tablet, a pill, a capsule, a liquid, a suspension, a gel, a dispersion, a solution, an emulsion, an ointment, or a lotion.

In one aspect, described herein is a method of treating a disease or condition in a mammal that would benefit by inhibition of low molecular weight protein tyrosine phosphatase (LMPTP) activity comprising administering to the mammal a compound disclosed herein, or a pharmaceutically acceptable salt or solvate thereof.

In some embodiments of the method of treating a disease or condition in a mammal that would benefit by inhibition of low molecular weight protein tyrosine phosphatase (LMPTP) activity, the mammal has type 2 diabetes, cardiovascular disease, coronary artery disease, hyperlipidemia, lipodystrophy, insulin resistance, rheumatic disease, atherosclerosis, myocardial infarction, stroke, high blood pressure (hypertension), obesity, elevated fasting plasma glucose, high serum triglycerides, elevated blood cholesterol, heart failure, metabolic syndrome, or a combination thereof.

In some embodiments of the method of treating a disease or condition in a mammal that would benefit by inhibition of low molecular weight protein tyrosine phosphatase (LMPTP) activity, the mammal has an impaired glucose tolerance.

In some embodiments of the method of treating a disease or condition in a mammal that would benefit by inhibition of low molecular weight protein tyrosine phosphatase (LMPTP) activity, the mammal is pre-diabetic.

In some embodiments of the method of treating a disease or condition in a mammal that would benefit by inhibition of low molecular weight protein tyrosine phosphatase (LMPTP) activity, the mammal is obese.

In some embodiments of the method of treating a disease or condition in a mammal that would benefit by inhibition of low molecular weight protein tyrosine phosphatase (LMPTP) activity, the compound modulates glucose and lipid metabolism.

In some embodiments of the method of treating a disease or condition in a mammal that would benefit by inhibition of low molecular weight protein tyrosine phosphatase (LMPTP) activity, the method further comprises administering an additional therapeutic agent to the mammal. In some embodiments of the method of treating a disease or condition in a mammal that would benefit by inhibition of low molecular weight protein tyrosine phosphatase (LMPTP) activity, the additional therapeutic agent is a peroxisome proliferator activated receptor (PPAR) agonist (gamma, dual, or pan), a dipeptidyl peptidase (IV) inhibitor, a glucagon-like peptide-1 (GLP-I) analog, insulin or an insulin analog, an insulin secretagogue, a sodium glucose co-transporter 2 (SGLT2) inhibitor, a human amylin analog, a biguanide, a glucophage, an alpha-glucosidase inhibitor, a meglitinide, a thiazolidinedione, a sulfonylurea, or any combination thereof. In some embodiments of the method of treating a disease or condition in a mammal that would benefit by inhibition of low molecular weight protein tyrosine phosphatase (LMPTP) activity, the additional therapeutic agent is an angiotensin-converting enzyme (ACE) inhibitor, angiotensin II receptor blocker (ARB), beta-blocker, diuretic, calcium channel blocker, inhibitor of renin-angiotensin system (RAS), blood-thinning medication, a statin, a fibrate, or any combination thereof.

In another aspect, described herein is a method of treating a metabolic disease or condition in a mammal comprising administering to the mammal a compound disclosed herein, or a pharmaceutically acceptable salt or solvate thereof.

In some embodiments of the method of treating a metabolic disease or condition, the metabolic disease or condition is mediated by low molecular weight protein tyrosine phosphatase (LMPTP) activity.

In some embodiments of the method of treating a metabolic disease or condition, the mammal has type 2 diabetes, cardiovascular disease, coronary artery disease, hyperlipidemia, lipodystrophy, insulin resistance, rheumatic disease, atherosclerosis, myocardial infarction, stroke, high blood pressure (hypertension), obesity, elevated fasting plasma glucose, high serum triglycerides, elevated blood cholesterol, heart failure, metabolic syndrome, or a combination thereof.

In some embodiments of a method of treating a metabolic disease or condition, the mammal has an impaired glucose tolerance.

In some embodiments of a method of treating a metabolic disease or condition, the mammal is pre-diabetic.

In some embodiments of a method of treating a metabolic disease or condition, the mammal is obese.

In some embodiments of a method of treating a metabolic disease or condition, the compound modulates glucose and lipid metabolism.

In some embodiments of a method of treating a metabolic disease or condition, the method further comprises administering an additional therapeutic agent to the mammal. In some embodiments of the method of treating a metabolic disease or condition, the additional therapeutic agent is a peroxisome proliferator activated receptor (PPAR) agonist (gamma, dual, or pan), a dipeptidyl peptidase (IV) inhibitor, a glucagon-like peptide-1 (GLP-I) analog, insulin or an insulin analog, an insulin secretagogue, a sodium glucose co-transporter 2 (SGLT2) inhibitor, a human amylin analog, a biguanide, a glucophage, an alpha-glucosidase inhibitor, a meglitinide, a thiazolidinedione, a sulfonylurea, or any combination thereof. In some embodiments of the method of treating a metabolic disease or condition, the additional therapeutic agent is an angiotensin-converting enzyme (ACE) inhibitor, angiotensin II receptor blocker (ARB), beta-blocker, diuretic, calcium channel blocker, inhibitor of renin-angiotensin system (RAS), blood-thinning medication, a statin, a fibrate, or any combination thereof.

In another aspect, described herein is a method of inhibiting low molecular weight protein tyrosine phosphatase (LMPTP) activity in a mammal comprising administering to the mammal a compound disclosed herein, or a pharmaceutically acceptable salt or solvate thereof.

In some embodiments of the method of inhibiting low molecular weight protein tyrosine phosphatase (LMPTP) activity, the mammal has type 2 diabetes, cardiovascular disease, coronary artery disease, hyperlipidemia, lipodystrophy, insulin resistance, rheumatic disease, atherosclerosis, myocardial infarction, stroke, high blood pressure (hypertension), obesity, elevated fasting plasma glucose, high serum triglycerides, elevated blood cholesterol, heart failure, metabolic syndrome, or a combination thereof.

In some embodiments of a method of inhibiting low molecular weight protein tyrosine phosphatase (LMPTP) activity, the mammal has an impaired glucose tolerance.

In some embodiments of a method of inhibiting low molecular weight protein tyrosine phosphatase (LMPTP) activity, the mammal is pre-diabetic.

In some embodiments of a method of inhibiting low molecular weight protein tyrosine phosphatase (LMPTP) activity, the mammal is obese.

In some embodiments of a method of inhibiting low molecular weight protein tyrosine phosphatase (LMPTP) activity, the compound modulates glucose and lipid metabolism.

In some embodiments of a method of inhibiting low molecular weight protein tyrosine phosphatase (LMPTP) activity, the method further comprises administering an additional therapeutic agent to the mammal. In some embodiments of a method of inhibiting low molecular weight protein tyrosine phosphatase (LMPTP) activity, the additional therapeutic agent is a peroxisome proliferator activated receptor (PPAR) agonist (gamma, dual, or pan), a dipeptidyl peptidase (IV) inhibitor, a glucagon-like peptide-1 (GLP-I) analog, insulin or an insulin analog, an insulin secretagogue, a sodium glucose co-transporter 2 (SGLT2) inhibitor, a human amylin analog, a biguanide, a glucophage, an alpha-glucosidase inhibitor, a meglitinide, a thiazolidinedione, a sulfonylurea, or any combination thereof. In some embodiments of a method of inhibiting low molecular weight protein tyrosine phosphatase (LMPTP) activity, the additional therapeutic agent is an angiotensin-converting enzyme (ACE) inhibitor, angiotensin II receptor blocker (ARB), beta-blocker, diuretic, calcium channel blocker, inhibitor of renin-angiotensin system (RAS), blood-thinning medication, a statin, a fibrate, or any combination thereof.

### DETAILED DESCRIPTION

Protein phosphorylation represents a key post-translational modification that is critical to the control of many cellular functions. The reversible phosphorylation of tyrosine residues of proteins is a significant regulatory event in eukaryotes compared to other protein phosphorylation processes and is crucially important for the regulation and progression of various cellular signaling cascades, especially those induced by receptor activation mechanisms. The appropriate functioning of these signaling pathways is controlled by the concerted and dynamic activities of protein tyrosine kinases (PTKs) and phosphotyrosine protein phosphatases (also known as protein tyrosine phosphatases, or PTPs) which play vital roles in numerous fundamental physiological cellular processes, such as growth, differentiation, survival, migration, metabolism, cell-cell communication and adhesion, immune response, and gene transcription.

PTPs are implicated in the pathogenesis of human diseases, including diabetes, obesity, cancer, inflammation, autoimmune, and cardiovascular diseases.

Low molecular weight PTPs (LMPTPs) have emerged as attractive targets for the pharmacological control of postreceptor events involved in the development of metabolic and neoplastic pathologies as well as for therapeutic intervention in infectious diseases.

LMPTP is a small (18 kD) cytosolic enzyme that is expressed ubiquitously but has particularly high expression in adipocytes. As a result of an alternative mRNA splicing mechanism, LMPTP is usually found as two isozymes, called LMPTP-A and -B (the rodent isoforms are called respectively LMPTP-IF1 and -IF2). In humans the total enzymatic activity of LMPTP is variable and is determined by a common genetic polymorphism.

LMPTPs have been identified and isolated from a wide variety of prokaryotic and eukaryotic organisms, such as bacteria, yeasts, and mammalians. LMPTPs from different organisms generally display a high degree of homology, especially in their tertiary structure.

Human LMPTPs exert sophisticated control over cell growth and differentiation through the modulation of signaling pathways induced by several growth factors and kinases. The enzyme also negatively regulates the metabolic responses to insulin, and the sensitivity of specific tissues to the hormone is consequently enhanced as a result of the LMPTP suppression.

LMPTP is an inhibitor of insulin signaling. In cell lines LMPTP is able to inhibit both the metabolic and growth-inducing effects of insulin. Also in vitro the phosphatase dephosphorylates peptides derived from the phosphorylated IGF-1 receptor and insulin receptor (IR). Increased insulin signaling was observed in the adipose tissue of obese mice treated with anti-LMPTP antisense oligonucleotides (ASO). It was shown that LMPTP can also easily be co-precipitated with the IR. Multiple lines of evidence suggest that LMPTP plays an important role in the metabolic syndrome. The first line of evidence comes from human genetic studies. The ACP1 gene is located in one of the candidate genome regions for obesity on chromosome 2p25 and is currently included in the obesity gene map. Carriers of ACP1 alleles associated with low enzymatic activity tend to have lower non-fasting glucose levels and are protected from obesity-associated lipid anomalies. Strong in vivo evidence suggesting that inhibition of LMPTP decreases the insulin resistance associated with obesity, by treating mice with anti-LMPTP ASOs. Leptin-deficient or diet-induced obese mice treated with specific anti-LMPTP ASOs showed a marked improvement of lipid profiles, and of glucose and insulin tolerance, in the absence of significant side effects.

Obesity is frequently complicated by a constellation of metabolic and cardiovascular anomalies, called the metabolic syndrome, which significantly increases morbidity and mortality of affected individuals. Insulin resistance is an important component of the metabolic syndrome. PTPs that regulate insulin signaling are targets for insulin resistance syndromes. One of the PTPs, the low molecular weight protein tyrosine phosphatase (LMPTP), is encoded by the ACP1 gene. LMPTP is highly expressed in adipocytes. There is strong in vitro and in vivo evidence that LMPTP is a negative regulator of insulin signaling. Genetic association studies in humans support a negative role for LMPTP in insulin resistance and the metabolic complications of obesity. In vivo, partial knock-down of LMPTP expression by specific antisense oligonucleotides (ASOs) led to improved glycemic and lipid profiles and decreased insulin resistance in diet-induced obese C57BL/6 mice. Interestingly, anti-LMPTP ASOs did not induce any metabolic phenotype in lean mice. LMPTP is considered to play a critical negative role in adipocyte insulin signaling, while it is less important in liver and muscle, where it can be at least partially compensated for by PTP1B, a critical negative regulator of insulin signaling in liver and skeletal muscle, and/or other prominent PTPs. Inhibition of LMPTP can significantly reduce obesity associated insulin resistance and decrease the severity of the metabolic syndrome in obesity.

It has been estimated that every year in the U.S. more than 70 billion dollars are spent for the treatment of obesity-related conditions and almost 300,000 deaths/year can be attributed to the complications of obesity. Obese patients often show multiple metabolic and cardiovascular anomalies known as “the metabolic syndrome”, including glucose intolerance, hyperlipidemia (especially high triglycerides with low HDL), and hypertension.

Obesity-induced insulin resistance is believed to be a central pathogenic factor in the metabolic syndrome. Obese patients are routinely treated with oral hypoglycemic agents, however even combinations of multiple agents are often insufficient to ensure adequate glycemic control, requiring the addition of parenteral insulin to the regimen. Reduced signal transduction at several levels after engagement of the insulin receptor (IR) has been observed in multiple insulin resistance syndromes, including the metabolic syndrome.

The IR is a protein tyrosine kinase, and tyrosine phosphorylation plays an important role in insulin signal transduction. Modification of the activity of the IR and/or tyrosine phosphorylation of IR targets are viewed as a promising way to reduce insulin resistance.

Provided herein are methods for improving insulin sensitivity in a subject comprising administering to the subject a LMPTP inhibitor; and thereby improving insulin sensitivity in the subject. In certain embodiments, the subject has insulin resistance. In some embodiments, the individual with insulin resistance has fasting insulin levels of at least 20 μU/mL. In some embodiments, the individual with insulin resistance has fasting insulin levels that exceed 100 μU/mL. In some embodiments, the LMPTP inhibitor treats a metabolic disorder by improving insulin resistance. In some embodiments, the LMPTP inhibitor treats a metabolic disorder by improving insulin sensitivity. In certain embodiments, the methods comprise selecting a subject having insulin resistance.

Provided herein are methods for treating metabolic disorders with a LMPTP inhibitor. The LMPTP inhibitor can treat, delay or prevent the onset of a metabolic disorder, wherein such metabolic disorders include, but are not limited to, metabolic syndrome, elevated blood glucose levels, insulin resistance, glucose intolerance, type 2 diabetes, type 1 diabetes, pre-diabetes, non-alcoholic fatty liver disease, nonalcoholic steatohepatitis, and obesity.

Insulin resistance may be detected using a procedure known as the hyperinsulinemic euglycemic clamp, which measures the amount of glucose necessary to compensate for an increased insulin level without causing hypoglycemia. In some embodiments, the methods disclosed herein comprise administering a LMPTP inhibitor to a subject with insulin resistance. In some embodiments, the LMPTP inhibitor improves insulin sensitivity. In some embodiments, the LMPTP inhibitor treats a metabolic disorder. In some embodiments, the LMPTP inhibitor treats a metabolic disorder by improving insulin sensitivity. In some embodiments, the LMPTP inhibitor delays or prevents the onset of the metabolic disorder by improving insulin sensitivity.

In some embodiments, described herein is a method of improving glucose tolerance in an individual comprising administering a LMPTP inhibitor to the subject with impaired glucose tolerance. In some embodiments, the individual has a metabolic disorder and the metabolic disorder is treated by improving glucose tolerance. In some embodiments, the LMPTP inhibitor delays or prevents the onset of a metabolic disorder in an individual by improving glucose tolerance.

In some embodiments, described herein is a method of treatment of a metabolic disorder in a subject that is overweight or obese. In some embodiments, a LMPTP inhibitor is used to treat obesity in a subject. In some embodiments, the LMPTP inhibitor decreases adipose tissue expansion in the subject that is overweight or obese. In some embodiments, the metabolic disorder is treated by decreasing adipose tissue expansion.

In some embodiments, administration of a LMPTP inhibitor to a subject delays or prevents the onset of a metabolic disorder by decreasing adipose tissue expansion. In some embodiments, the subject is at risk for developing a metabolic disorder.

### Compounds

Described herein are small molecule LMPTP inhibitors. In one aspect, described herein is a compound of Formula (I), or a pharmaceutically acceptable salt or solvate thereof:

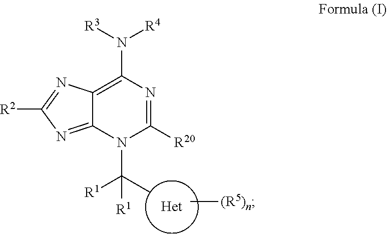

wherein:
Ring Het is heteroaryl;
each R¹ is independently hydrogen, —F, or —CH₃;
R² is halogen, —CN, —OH, —OR^{a}, —SH, —SR^{a}, C₁-C₆ alkyl, C₂-C₆ alkenyl, C₂-C₆ alkynyl, or C₁-C₆ heteroalkyl; wherein alkyl, alkenyl, alkynyl and C₁-C₆ heteroalkyl is unsubstituted or substituted with one, two, or three R⁶;
   each R⁶ is independently halogen, —CN, —OH, —OR^{a}, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —C(═O)N(R^{b})₂, —NR^{b}C(═O)R^{a};
or R² is

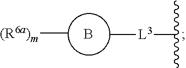

L³ is absent or C₁-C₆ alkylene;
Ring B is phenyl, heteroaryl, cycloalkyl, or heterocycloalkyl;
each R^{6a} is independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(O)R^{a}, —NR^{b}C(═O)OR^{b}, C₁-C₆ alkyl, C₁-C₆ haloalkyl, C₁-C₆ hydroxyalkyl, C₁-C₆ heteroalkyl, or cycloalkyl which is unsubstituted or substituted with one, two, or three halogen, C₁-C₆ alkyl, or C₁-C₆ haloalkyl;
or two R^{6a} groups join together with the intervening atoms of ring B that connect the two R^{6a} groups to form a ring that is unsubstituted or substituted with one, two, or three halogen, —CN, —OH, C₁-C₆ alkyl, C₁-C₆ alkoxy, C₁-C₆ haloalkyl or C₁-C₆ haloalkoxy;
m is 0, 1, 2, 3, 4, or 5;
R³ is hydrogen, C₁-C₆ alkyl, C₂-C₆ alkenyl, C₂-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are independently unsubstituted or substituted with one, two, or three R¹⁰;
R⁴ is hydrogen, C₁-C₆ alkyl, C₂-C₆ alkenyl, C₂-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are independently unsubstituted or substituted with one, two, or three R¹¹;
or R⁴ is -L¹-L²-R⁷;
   L¹ is —C(═O)—, —S(═O)—, —S(═O)₂—, or C₁-C₄ alkylene;
   L² is absent or —CH₂—;
   R⁷ is C₁-C₆ alkyl, C₂-C₆ alkenyl, C₂-C₆ alkynyl, cycloalkyl, heterocycloalkyl, aryl, monocyclic heteroaryl that contains 1-4 N atoms and 0-2 O or S atoms, monocyclic heteroaryl that contains 0-4 N atoms and 1 S atom, or bicyclic heteroaryl that contains 0-4 N atoms and 0-2 O or S atoms; wherein each alkyl, alkenyl, alkynyl, cycloalkyl, heterocycloalkyl, aryl, and heteroaryl is unsubstituted or substituted with one, two, or three R⁸;
each R⁸ is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, C₁-C₆ heteroalkyl, or cycloalkyl which is unsubstituted or substituted with one, two, or three halogen, C₁-C₆ alkyl, or C₁-C₆ haloalkyl;
or R³ and R⁴ are taken together with the nitrogen atom to which they are attached to form a heterocycloalkyl which is unsubstituted or substituted with one, two, or three R¹²;
each R⁵ is independently hydrogen, halogen, —CN, —OH, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, C₁-C₆ heteroalkyl, or cycloalkyl which is unsubstituted or substituted with one, two, or three halogen, C₁-C₆ alkyl, or C₁-C₆ haloalkyl;
n is 0, 1, 2, 3, 4, or 5;
each R¹⁰, R¹¹, and R¹² is independently halogen, —CN, —OH, —SH, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, C₁-C₆ heteroalkyl, or cycloalkyl which is unsubstituted or substituted with one, two, or three halogen, C₁-C₆ alkyl, or C₁-C₆ haloalkyl;
R²⁰ is hydrogen, halogen, —CN, —OH, —SH, —SR^{a}, C₁-C₆ alkyl, C₁-C₆ haloalkyl, C₁-C₆ hydroxyalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl; and
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl;
or two R^{b} groups on a nitrogen atom are taken together with the nitrogen atom to which they are attached to form a heterocycloalkyl which is unsubstituted or substituted with one, two, or three halogen, C₁-C₆ alkyl, or C₁-C₆ haloalkyl.

For any and all of the embodiments, substituents are selected from among a subset of the listed alternatives. For example, in some embodiments, each R¹ is hydrogen, —F, or —CH₃. In some embodiments, each R¹ is hydrogen. In some embodiments, each R¹ is —F. In some embodiments, each R¹ is —CH₃. In some embodiments, one R¹ is —CH₃ and the other R¹ is hydrogen.

In some embodiments, the compound of Formula (I) is not 3-[(3,5-dichloro(4-pyridyl))methyl]-8-phenyl-3-hydropurine-6-ylamine. In some embodiments, at least one R¹ is not hydrogen if R² is phenyl, R³ is hydrogen, R⁴ is hydrogen, R²⁰ is hydrogen, and Ring Het is pyrid-4-yl. In some embodiments, at least one R¹ is not hydrogen if R² is phenyl, R³ is hydrogen, R⁴ is hydrogen, R²⁰ is hydrogen, and

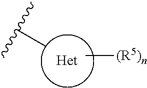

is (3,5-dichloro(pyrid-4-yl). In some embodiments, at least one R^{6a} group is not hydrogen if Ring B is phenyl.

In some embodiments, R² is

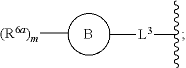

L³ is absent or C₁-C₆ alkylene; Ring B is phenyl, heteroaryl, cycloalkyl, or heterocycloalkyl; each R^{6a} is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(O)R^{a}, —NR^{b}C(O)OR^{b}, C₁-C₆ alkyl, C₁-C₆ haloalkyl, C₁-C₆ hydroxyalkyl, C₁-C₆ heteroalkyl, or cycloalkyl which is unsubstituted or substituted with one, two, or three halogen, C₁-C₆ alkyl, or C₁-C₆ haloalkyl; or two R^{6a} groups join together with the intervening atoms of Ring B that connect the two R^{6a} groups to form a ring that is unsubstituted or substituted with one, two, or three halogen, —CN, —OH, C₁-C₆ alkyl, C₁-C₆ alkoxy, C₁-C₆ haloalkyl or C₁-C₆ haloalkoxy; m is 1, 2, 3, 4, or 5.

In some embodiments, Ring B is heteroaryl, cycloalkyl, or heterocycloalkyl. In some embodiments, R² is not phenyl.

In some embodiments, described herein is a compound of Formula (I), or a pharmaceutically acceptable salt or solvate thereof, wherein:
Ring Het is a monocyclic heteroaryl;
each R¹ is hydrogen, —F, or —CH₃;
R² is halogen, —CN, —OR^{a}, C₁-C₆ alkyl, or C₁-C₆ heteroalkyl;
or R² is

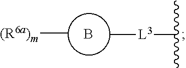

L³ is absent or —CH₂—;
Ring B is phenyl, or monocyclic heteroaryl;
each R^{6a} is independently hydrogen, —F, —Cl, —CN, —OMe, —OCF₃, —CH₃, or —CF₃;
m is 0, 1, or 2;
R³ is hydrogen, or C₁-C₆ alkyl;
R⁴ is hydrogen, C₁-C₆ alkyl, cycloalkyl, or phenyl; wherein the alkyl, cycloalkyl, or phenyl is unsubstituted or substituted with one, two, or three R¹¹;
or R⁴ is -L¹-L²-R⁷;
   L¹ is —C(═O)—, —S(═O)₂—, or —CH₂—;
   L² is absent or —CH₂—;
   R⁷ is C₁-C₆ alkyl, aryl, heteroaryl that contains 1-4 N atoms and 0-2 O or S atoms, or heteroaryl that contains 0-4 N atoms and 1 S atom; wherein each alkyl, aryl, and heteroaryl is unsubstituted or substituted with one, two, or three R⁸;
   each R⁸ is independently —F, —Cl, —CN, —OMe, or methyl;
or R³ and R⁴ are taken together with the nitrogen atom to which they are attached to form a heterocycloalkyl;
each R⁵ is independently hydrogen, —F, —Cl, —Br, —CN, —OMe, —CH₃, or —CF₃;
n is 1-3;
each R¹¹ is independently halogen, —CN, —OH, —OR^{a}, —NO₂, —N(R^{b})₂, —C(═O)R^{a}, —C(═O)OR^{b}, —C(═O)N(R^{b})₂, C₁-C₆ alkyl, or C₁-C₆ haloalkyl;
R²⁰ is hydrogen or C₁-C₆ alkyl;
each R^{a} is independently C₁-C₆ alkyl, C₁-C₆ heteroalkyl, cycloalkyl, heterocycloalkyl, aryl, or heteroaryl; wherein the alkyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl; and
each R^{b} is independently hydrogen, C₁-C₆ alkyl, C₁-C₆ heteroalkyl, cycloalkyl, heterocycloalkyl, aryl, or heteroaryl; wherein the alkyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl;
or two R^{b} groups on a nitrogen atom are taken together with the nitrogen atom to which they are attached to form a heterocycloalkyl which is unsubstituted or substituted with one, two, or three halogen, C₁-C₆ alkyl, or C₁-C₆ haloalkyl.

In some embodiments, described herein is a compound of Formula (I), or a pharmaceutically acceptable salt or solvate thereof, wherein:
Ring Het is a 5- or 6-membered heteroaryl;
each R¹ is hydrogen;
R² is halogen, —CN, —OR^{a}, C₁-C₆ alkyl or C₁-C₆ heteroalkyl;
or R² is

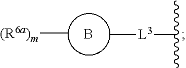

L³ is absent or —CH₂—;
Ring B is phenyl, or monocyclic heteroaryl;
each R^{6a} is independently hydrogen, —F, —Cl, —CN, —OMe, —OCF₃, —CH₃, or —CF₃;
m is 0 or 1;
R³ is hydrogen, or C₁-C₆ alkyl;
R⁴ is hydrogen, C₁-C₆ alkyl, cycloalkyl, or phenyl; wherein the alkyl, cycloalkyl, or phenyl is unsubstituted or substituted with one, two, or three R¹¹;
or R⁴ is -L¹-L²-R⁷;
   L¹ is —C(═O)—, —S(═O)₂—, or —CH₂—;
   L² is absent or —CH₂—;
   R⁷ is C₁-C₆ alkyl, aryl, heteroaryl that contains 1-4 N atoms and 0-2 O or S atoms, or heteroaryl that contains 0-4 N atoms and 1 S atom; wherein each alkyl, aryl, and heteroaryl is unsubstituted or substituted with one, two, or three R⁸;
   each R⁸ is independently —F, —Cl, —CN, —OMe, or methyl;
or R³ and R⁴ are taken together with the nitrogen atom to which they are attached to form a heterocycloalkyl;
each R⁵ is independently hydrogen, —F, —Cl, —Br, —CN, —OMe, —CH₃, or —CF₃;
n is 1-3;
each R¹¹ is independently halogen, —CN, —OH, —OR^{a}, —NO₂, —N(R^{b})₂, —C(═O)R^{a}, —C(═O)OR^{b}, —C(═O)N(R^{b})₂, C₁-C₆ alkyl, or C₁-C₆ haloalkyl;
R²⁰ is hydrogen;
each R^{a} is independently C₁-C₆ alkyl, C₁-C₆ heteroalkyl, cycloalkyl, heterocycloalkyl, aryl, or heteroaryl; wherein the alkyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl; and
each R^{b} is independently hydrogen, C₁-C₆ alkyl, C₁-C₆ heteroalkyl, cycloalkyl, heterocycloalkyl, aryl, or heteroaryl; wherein the alkyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl;
or two R^{b} groups on a nitrogen atom are taken together with the nitrogen atom to which they are attached to form a heterocycloalkyl which is unsubstituted or substituted with one, two, or three halogen, C₁-C₆ alkyl, or C₁-C₆ haloalkyl.

In some embodiments, the compound of Formula (I) has the following structure:

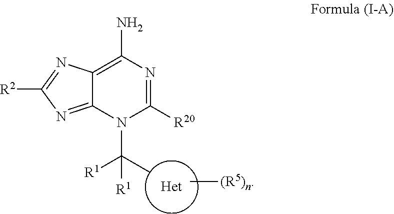

In some embodiments, the compound of Formula (I) has the structure of Formula (I-A), or a pharmaceutically acceptable salt or solvate thereof:

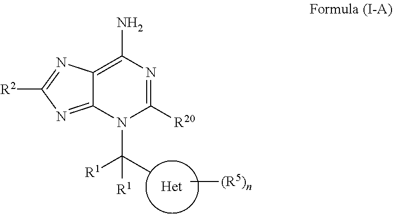

wherein:
Ring Het is a 5-membered or 6-membered heteroaryl;
each R¹ is hydrogen;
R² is

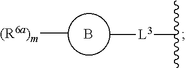

L³ is absent or C₁-C₆ alkylene;
Ring B is phenyl, monocyclic heteroaryl, cycloalkyl, or heterocycloalkyl;
each R^{6a} is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, C₁-C₆ heteroalkyl, or cycloalkyl which is unsubstituted or substituted with one, two, or three halogen, C₁-C₆ alkyl, or C₁-C₆ haloalkyl;
or two R^{6a} groups join together with the intervening atoms of ring B that connect the two R^{6a} groups to form a ring that is unsubstituted or substituted with one, two, or three halogen, —CN, —OH, C₁-C₆ alkyl, C₁-C₆ alkoxy, C₁-C₆ haloalkyl or C₁-C₆ haloalkoxy;
m is 1, 2, 3, 4, or 5;
each R⁵ is independently hydrogen, halogen, —CN, —OH, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, C₁-C₆ heteroalkyl, or cycloalkyl which is unsubstituted or substituted with one, two, or three halogen, C₁-C₆ alkyl, or C₁-C₆ haloalkyl;
n is 0, 1, 2, 3, 4, or 5;
R²⁰ is hydrogen, halogen, C₁-C₆ alkyl, C₁-C₆ haloalkyl, or C₁-C₆ heteroalkyl;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl; and
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl;
or two R^{b} groups on a nitrogen atom are taken together with the nitrogen atom to which they are attached to form a heterocycloalkyl which is unsubstituted or substituted with one, two, or three halogen, C₁-C₆ alkyl, or C₁-C₆ haloalkyl.

In some embodiments, L³ is absent, or —CH₂—; Ring B is phenyl, pyridinyl, pyrazinyl, pyrimidinyl, pyridazinyl, triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl; Ring Het is pyridinyl, pyrazinyl, pyrimidinyl, pyridazinyl, triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl; and R²⁰ is hydrogen;

In some embodiments, Ring B is phenyl or pyridinyl; and Ring Het is pyridinyl or pyrazinyl.

In some embodiments, each R^{6a} is independently halogen, —CN, —OH, —OR^{a}, —N(R^{b})₂, —C₁-C₆ alkyl, or C₁-C₆ haloalkyl. In some embodiments, each R^{6a} is independently hydrogen, —F, —Cl, —Br, —CH₃, —NH₂, or —CF₃.

In some embodiments, each R⁵ is independently hydrogen, —F, —Cl, —Br, —I, —CN, —OMe, —OCF₃, —OEt, —OiPr, —NH₂, —NMe₂, —C(═O)Me, —C(═O)OH, —C(═O)OMe, —C(═O)OEt, —C(═O)NH₂, —C(═O)NMe₂, —CH₃, —CH₂CH₃, —CH₂CH₂CH₃, —CH(CH₃)₂, or —CF₃. In some embodiments, each R⁵ is independently hydrogen, halogen, —CN, —OH, —OR^{a}, or —N(R^{b})₂. In some embodiments, each R⁵ is independently hydrogen, —F, —Cl, —Br, —I, —OMe, —CH₃, or —CF₃. In some embodiments, each R⁵ is independently hydrogen, —Br, or —Cl.

In some embodiments, R²⁰ is hydrogen, halogen, —CN, —OR^{a}, —SR^{a}, C₁-C₆ alkyl, C₁-C₆ haloalkyl, or C₁-C₆ heteroalkyl. In some embodiments, R²⁰ is hydrogen, —F, —Cl, —Br, —CN, C₁-C₆ alkyl, or C₁-C₆ haloalkyl. In some embodiments, R²⁰ is hydrogen, —F, —Cl, or —CN. In some embodiments, R²⁰ is hydrogen.

In some embodiments, each R¹ is hydrogen; and R²⁰ is hydrogen, halogen, —CN, —OR^{a}, —SR^{a}, C₁-C₆ alkyl, C₁-C₆ haloalkyl, or C₁-C₆ heteroalkyl.

In some embodiments, each R¹ is hydrogen; and R²⁰ is hydrogen.

In some embodiments, R³ is hydrogen, C₁-C₆ alkyl, cycloalkyl, heterocycloalkyl, aryl, or heteroaryl; wherein alkyl, cycloalkyl, heterocycloalkyl, aryl and heteroaryl are independently unsubstituted or substituted with one, two, or three R¹⁰. In some embodiments, R³ is hydrogen, C₁-C₆ alkyl, aryl, or heteroaryl; wherein alkyl, aryl and heteroaryl are independently unsubstituted or substituted with one, two, or three R¹⁰. In some embodiments, R³ is hydrogen, or C₁-C₆ alkyl which is unsubstituted or substituted with one, two, or three R¹⁰. In some embodiments, R³ is hydrogen or C₁-C₆ alkyl. In some embodiments, R³ is hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, or —C(CH₃)₃. In some embodiments, R³ is hydrogen, —CH₃, —CH₂CH₃, —CH₂CH₂CH₃, —CH(CH₃)₂, or —C(CH₃)₃. In some embodiments, R³ is hydrogen, or —CH₃. In some embodiments, R³ is hydrogen. In some embodiments, R³ is —CH₃.

In some embodiments, each R¹⁰ is independently halogen, —CN, —OH, —OR^{a}, —NO₂, —N(R^{b})₂, —C(═O)R^{a}, —C(═O)OR^{b}, —C(═O)N(R^{b})₂, C₁-C₆ alkyl, or C₁-C₆ haloalkyl. In some embodiments, each R¹⁰ is independently —F, —Cl, —Br, —OH, —CN, —OMe, —OBn, —OCF₃, —OCHF₂, —NO₂, —NH₂, —NMe₂, piperidinyl, morpholinyl, —C(═O)Me, —C(═O)OH, —C(═O)OMe, —C(═O)NH₂, —C(═O)NMe₂, methyl, ethyl, propyl, tert-butyl, or —CF₃.

In some embodiments, R⁴ is hydrogen, C₁-C₆ alkyl, cycloalkyl, heterocycloalkyl, aryl, or heteroaryl; wherein alkyl, cycloalkyl, heterocycloalkyl, aryl and heteroaryl are independently unsubstituted or substituted with one, two, or three R¹¹; or R⁴ is -L¹-L²-R⁷.

In some embodiments, R⁴ is hydrogen, C₁-C₆ alkyl, cycloalkyl, heterocycloalkyl, aryl, or heteroaryl; wherein alkyl, cycloalkyl, heterocycloalkyl, aryl and heteroaryl are independently unsubstituted or substituted with one, two, or three R¹¹. In some embodiments, R⁴ is C₁-C₆ alkyl, cycloalkyl, or aryl; wherein the alkyl, cycloalkyl, and aryl is unsubstituted or substituted with one, two, or three R¹¹. In some embodiments, R⁴ is C₁-C₆ alkyl, cycloalkyl, or phenyl; wherein the alkyl, cycloalkyl, and phenyl is unsubstituted or substituted with one, two, or three R¹¹.

In some embodiments, R⁴ is C₁-C₆ alkyl which is unsubstituted or substituted with one, two, or three R¹¹. In some embodiments, R⁴ is C₁-C₆ alkyl which is unsubstituted or substituted with one R¹¹. In some embodiments, R⁴ is C₁-C₆ alkyl which is unsubstituted or substituted with two R¹¹. In some embodiments, R⁴ is C₁-C₆ alkyl which is unsubstituted or substituted with three R¹¹. In some embodiments, R⁴ is methyl, ethyl, propyl, butyl, or pentyl; wherein the methyl, ethyl, propyl, butyl, or pentyl is unsubstituted or substituted with one R¹¹.

In some embodiments, R⁴ is cycloalkyl which is unsubstituted or substituted with one, two, or three R¹¹. In some embodiments, R⁴ is cycloalkyl which is unsubstituted or substituted with one R¹¹. In some embodiments, R⁴ is cycloalkyl which is unsubstituted or substituted with two R¹¹. In some embodiments, R⁴ is cycloalkyl which is unsubstituted or substituted with three R¹¹. In some embodiments, R⁴ is cyclopropyl, cyclobutyl, cyclopentyl, or cyclohexyl. In some embodiments, R⁴ is cyclopropyl.

In some embodiments, R⁴ is phenyl which is unsubstituted or substituted with one, two, or three R¹¹. In some embodiments, R⁴ is phenyl which is unsubstituted or substituted with one R¹¹. In some embodiments, R⁴ is phenyl which is unsubstituted or substituted with two R¹¹. In some embodiments, R⁴ is phenyl which is unsubstituted or substituted with three R¹¹.

In some embodiments, each R¹¹ is independently halogen, —CN, —OH, —OR^{a}, —NO₂, —N(R^{b})₂, —C(═O)R^{a}, —C(═O)OR^{b}, —C(═O)N(R^{b})₂, C₁-C₆ alkyl, or C₁-C₆ haloalkyl. In some embodiments, each R¹¹ is independently —F, —Cl, —Br, —OH, —CN, —OMe, —OBn, —OCF₃, —OCHF₂, —NO₂, —NH₂, —NMe₂, piperidinyl, morpholinyl, —C(═O)Me, —C(═O)OH, —C(═O)OMe, —C(═O)NH₂, —C(═O)NMe₂, methyl, ethyl, propyl, tert-butyl, or —CF₃.

In some embodiments, R⁴ is -L¹-L²-R⁷.

In some embodiments, L¹ is —C(═O)—. In some embodiments, L¹ is —S(═O)—. In some embodiments, L¹ is —S(═O)₂—. In some embodiments, L¹ is C₁-C₄ alkylene. In some embodiments, L¹ is C₁-C₂ alkylene. In some embodiments, L¹ is —CH₂—. In some embodiments, L¹ is —C(═O)—, —S(═O)₂—, or —CH₂—.

In some embodiments, L² is absent. In some embodiments, L² is —CH₂—.

In some embodiments, L¹ is —CH₂— and L² is absent. In some embodiments, L¹ is —C(═O)— and L² is absent. In some embodiments, L¹ is —S(═O)₂— and L² is absent. In some embodiments, L¹ is —S(═O)₂— and L² is —CH₂—.

In some embodiments, R⁷ is C₁-C₆ alkyl, C₂-C₆ alkenyl, C₂-C₆ alkynyl, cycloalkyl, heterocycloalkyl, aryl, monocyclic heteroaryl that contains 1-4 N atoms and 0-2 O or S atoms, monocyclic heteroaryl that contains 0-4 N atoms and 1 S atom, or bicyclic heteroaryl that contains 0-4 N atoms and 0-2 O or S atoms; wherein each alkyl, alkenyl, alkynyl, cycloalkyl, heterocycloalkyl, aryl and heteroaryl is unsubstituted or substituted with one, two, or three R⁸. In some embodiments, R⁷ is C₁-C₆ alkyl, cycloalkyl, heterocycloalkyl, aryl, heteroaryl that contains 1-4 N atoms and 0-2 O or S atoms, or heteroaryl that contains 0-4 N atoms and 1 S atom; wherein each alkyl, cycloalkyl, heterocycloalkyl, aryl and heteroaryl is unsubstituted or substituted with one, two, or three R⁸. In some embodiments, R⁷ is C₁-C₆ alkyl, aryl, heteroaryl that contains 1-4 N atoms and 0-2 O or S atoms, or heteroaryl that contains 0-4 N atoms and 1 S atom; wherein each alkyl, aryl, and heteroaryl is unsubstituted or substituted with one, two, or three R⁸. In some embodiments, R⁷ is methyl, ethyl, phenyl, pyridyl, oxazolyl, or thienyl; wherein the phenyl, pyridyl, oxazolyl, and thienyl are unsubstituted, or substituted with one, two, or three R⁸. In some embodiments, R⁷ is methyl, ethyl, phenyl, pyridyl, oxazolyl, or thienyl. In some embodiments, R⁷ is methyl, ethyl, phenyl, pyridyl, oxazolyl, or thienyl; wherein the phenyl, pyridyl, oxazolyl, and thienyl are substituted with one R⁸. In some embodiments, R⁷ is methyl, ethyl, phenyl, pyridyl, oxazolyl, or thienyl; wherein the phenyl, pyridyl, oxazolyl, and thienyl are substituted with two R⁸. In some embodiments, R⁷ is methyl, ethyl, phenyl, pyridyl, oxazolyl, or thienyl; wherein the phenyl, pyridyl, oxazolyl, and thienyl are substituted with three R⁸.

In some embodiments, each R⁸ is independently halogen, —CN, —OH, —OR^{a}, —NO₂, —N(R^{b})₂, —C(═O)R^{a}, —OC(═O)R^{a}, —C(═O)OR^{b}, —C(═O)N(R^{b})₂, C₁-C₆ alkyl, or C₁-C₆haloalkyl. In some embodiments, each R⁸ is independently halogen, —CN, —OH, —OR^{a}, —NO₂, —N(R^{b})₂, —C(═O)R^{a}, —C(═O)OR^{b}, —C(═O)N(R^{b})₂, C₁-C₆ alkyl, or C₁-C₆haloalkyl. In some embodiments, each R⁸ is independently —F, —Cl, —CN, —OMe, or methyl.

In some embodiments, R³ is hydrogen. In some embodiments, R⁴ is hydrogen. In some embodiments, R³ is hydrogen; and R⁴ is hydrogen.

In some embodiments, R³ and R⁴ are taken together with the nitrogen atom to which they are attached to form a heterocycloalkyl which is unsubstituted or substituted with one, two, or three R¹². In some embodiments, R³ and R⁴ are taken together with the nitrogen atom to which they are attached to form a 3- to 7-membered heterocycloalkyl which is unsubstituted or substituted with one, two, or three R¹². In some embodiments, R³ and R⁴ are taken together with the nitrogen atom to which they are attached to form a 5- or 6-membered heterocycloalkyl which is unsubstituted or substituted with one, two, or three R¹². In some embodiments, R³ and R⁴ are taken together with the nitrogen atom to which they are attached to form a pyrrolidine, piperidine, or morpholine which is unsubstituted or substituted with one, two, or three R¹². In some embodiments, R³ and R⁴ are taken together with the nitrogen atom to which they are attached to form a pyrrolidine, piperidine, or morpholine.

In some embodiments, each R¹² is independently halogen, —CN, —OH, —OR^{a}, —N(R^{b})₂, —C(═O)R^{a}, —C(═O)OR^{b}, —C(═O)N(R^{b})₂, C₁-C₆ alkyl, or C₁-C₆ haloalkyl.

In some embodiments, Ring Het is a monocyclic heteroaryl.

In some embodiments, Ring Het is a 5- or 6-membered heteroaryl.

In some embodiments, Ring Het is pyridinyl, pyrazinyl, pyrimidinyl, pyridazinyl, triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl.

In some embodiments, Ring Het is a 6-membered heteroaryl. In some embodiments, Ring Het is a 6-membered heteroaryl that is pyridinyl, pyrazinyl, pyrimidinyl, or pyridazinyl. In some embodiments, Ring Het is a 6-membered heteroaryl that is pyridinyl, pyrazinyl, or pyrimidinyl. In some embodiments, Ring Het is a 6-membered heteroaryl that is pyridinyl. In some embodiments, Ring Het is a 6-membered heteroaryl that is pyrazinyl. In some embodiments, Ring Het is a 6-membered heteroaryl that is pyrimidinyl. In some embodiments, Ring Het is a 6-membered heteroaryl that is pyridazinyl.

In some embodiments,

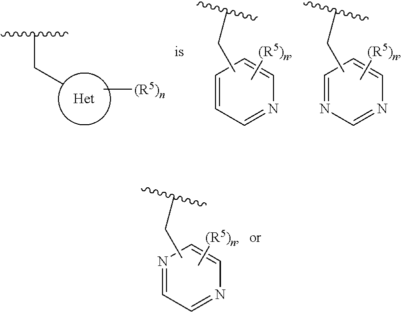

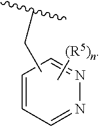

In some embodiments,

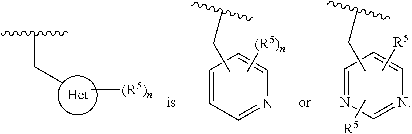

In some embodiments,

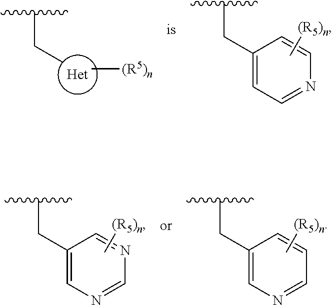

In some embodiments,

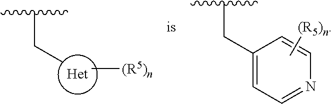

In some embodiments,

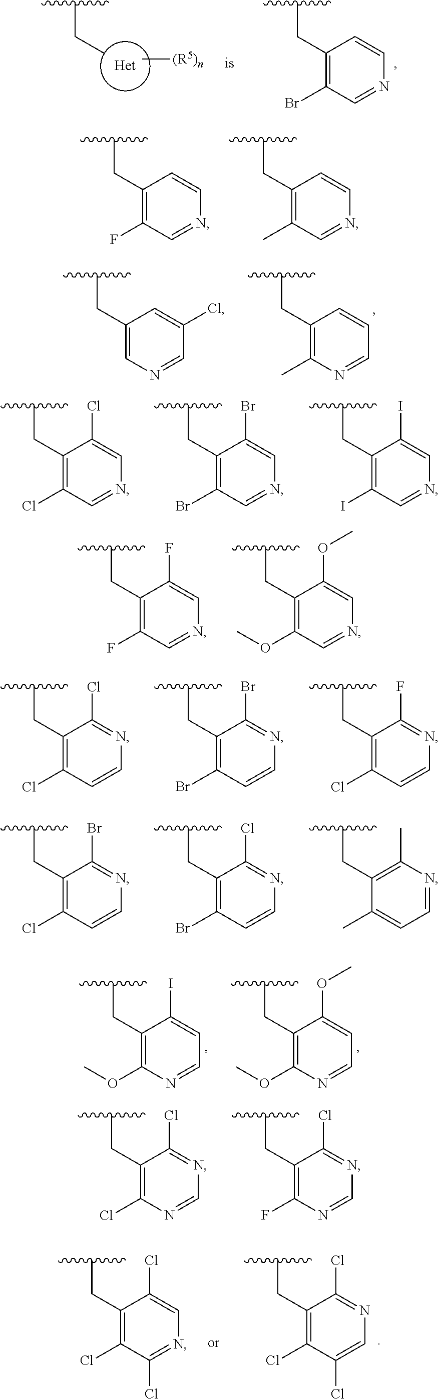

In some embodiments,

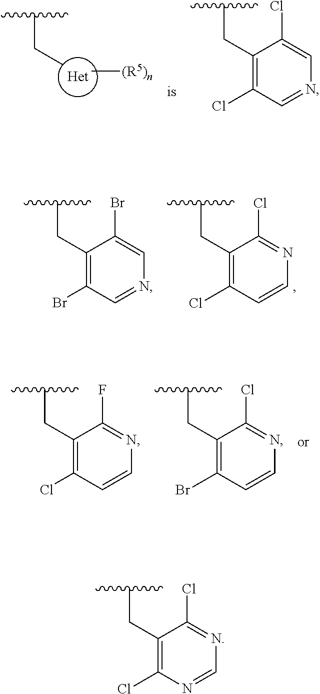

In some embodiments,

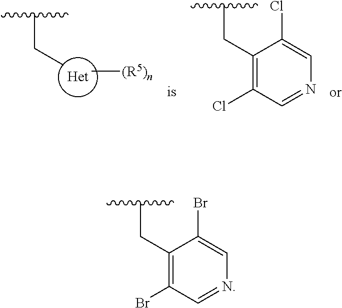

In some embodiments, the compound of Formula (I) or Formula (I-A) has the structure of Formula (II), or a pharmaceutically acceptable salt or solvate thereof:

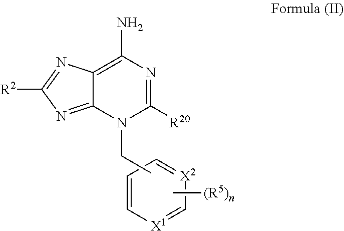

wherein:
X¹ is CH or N; and
X² is CH or N.

In some embodiments, the compound of Formula (II) has the structure of Formula (M-A) or Formula (III-B), or a pharmaceutically acceptable salt or solvate thereof:

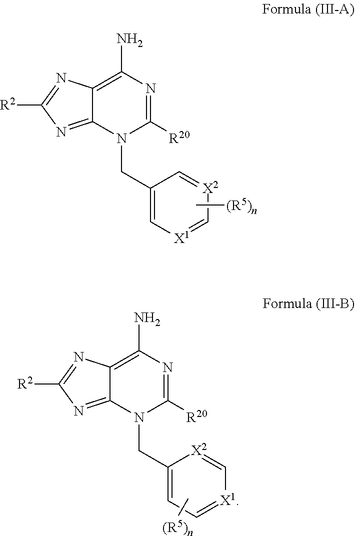

In some embodiments, the compound of Formula (II) has the structure of Formula (IV-A), (IV-B), or (IV-C), or a pharmaceutically acceptable salt or solvate thereof:

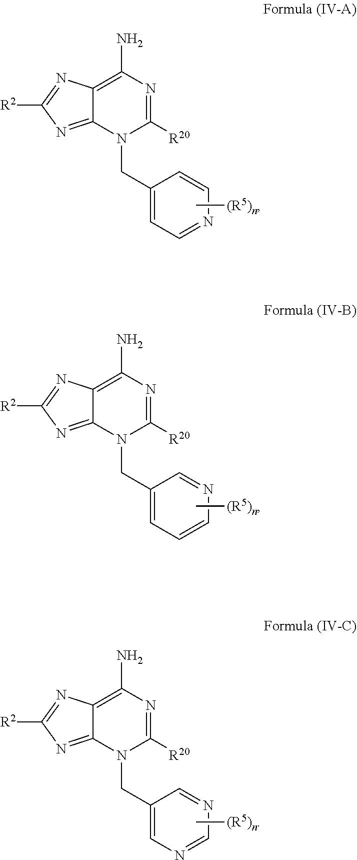

In some embodiments, Ring Het is a 5-membered heteroaryl. In some embodiments, Ring Het is a 5-membered heteroaryl that is triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl. In some embodiments, Ring Het is a 5-membered heteroaryl that is imidazolyl, pyrazolyl, thiophenyl, thiazolyl, isothiazolyl, triazolyl, or thiadiazolyl. In some embodiments, Ring Het is a 5-membered heteroaryl that is imidazolyl, thiophenyl, triazolyl, or thiadiazolyl. In some embodiments, Ring Het is a 5-membered heteroaryl that is imidazolyl. In some embodiments, Ring Het is a 5-membered heteroaryl that is thiophenyl. In some embodiments, Ring Het is a 5-membered heteroaryl that is triazolyl. In some embodiments, Ring Het is a 5-membered heteroaryl that is thiadiazolyl.

In some embodiments,

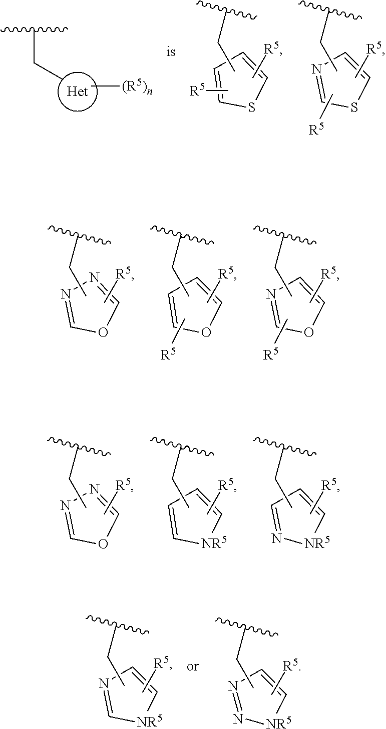

In some embodiments,

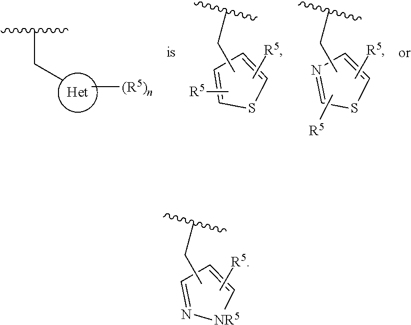

In some embodiments,

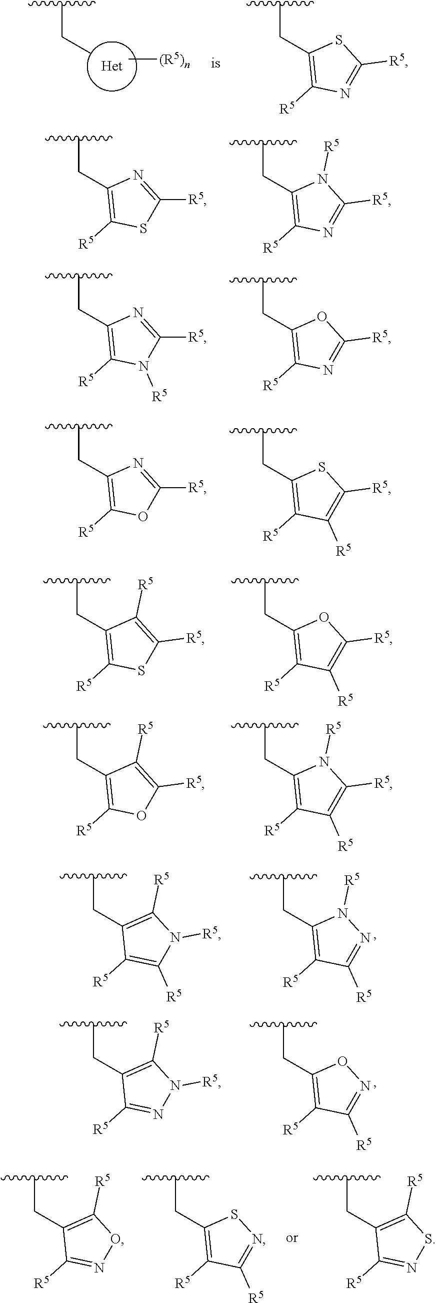

In some embodiments,

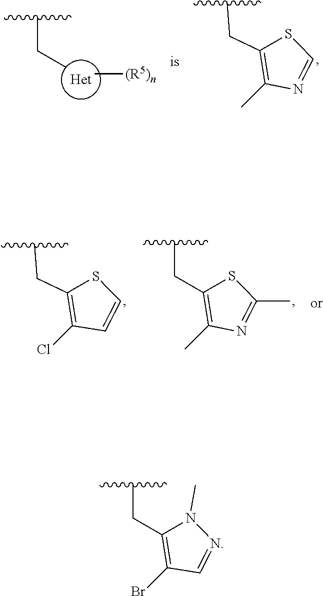

In some embodiments,

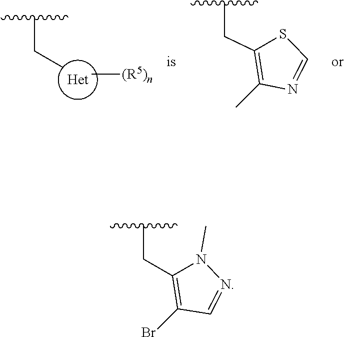

In some embodiments,

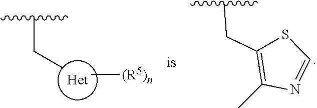

In some embodiments,

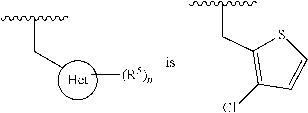

In some embodiments,

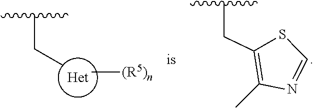

In some embodiments,

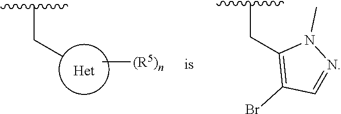

In some embodiments, the compound of Formula (I) or Formula (I-A) has the structure of Formula (V), or a pharmaceutically acceptable salt or solvate thereof:

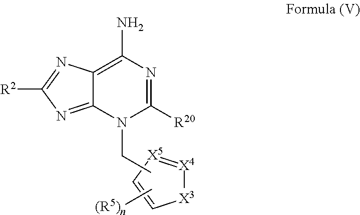

wherein,
X³ is CR⁵, CH, N, S, or O;
X⁴ is CR⁵, CH, or N; and
X⁵ is CR⁵, CH, or N.

In some embodiments, the compound of Formula (I) or Formula (I-A) has the structure of Formula (V-A) of Formula (V-B), or a pharmaceutically acceptable salt or solvate thereof:

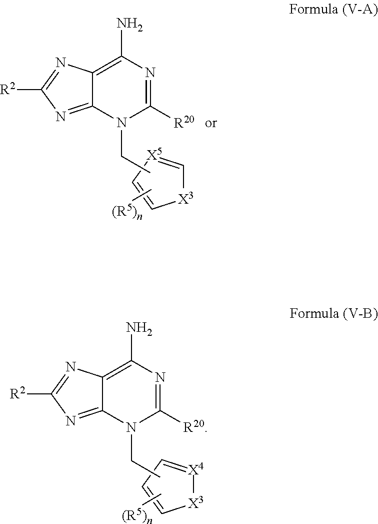

In some embodiments, the compound of Formula (I) or Formula (I-A) has the structure of Formula (VI-A), (VI-B), (VI-C), or (VI-D), or a pharmaceutically acceptable salt or solvate thereof:

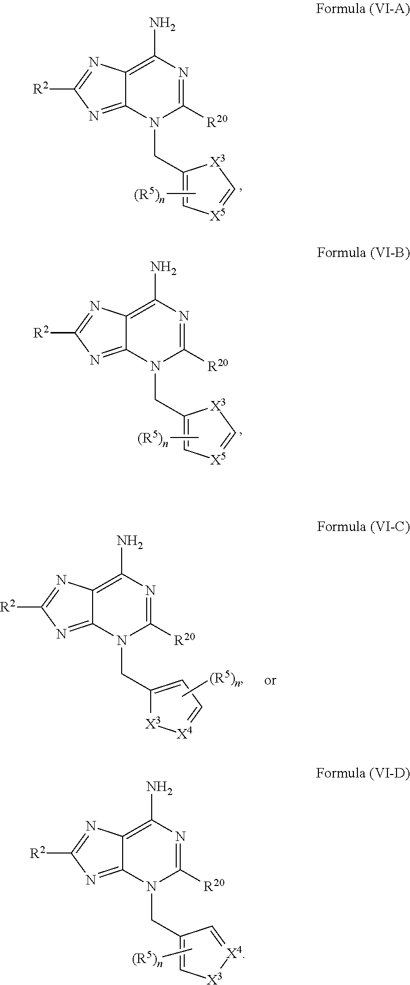

In some embodiments, the compound of Formula (I) or Formula (I-A) has the structure of Formula (VA-1), (VA-2), (VA-3), or (VA-4); or a pharmaceutically acceptable salt or solvate thereof:

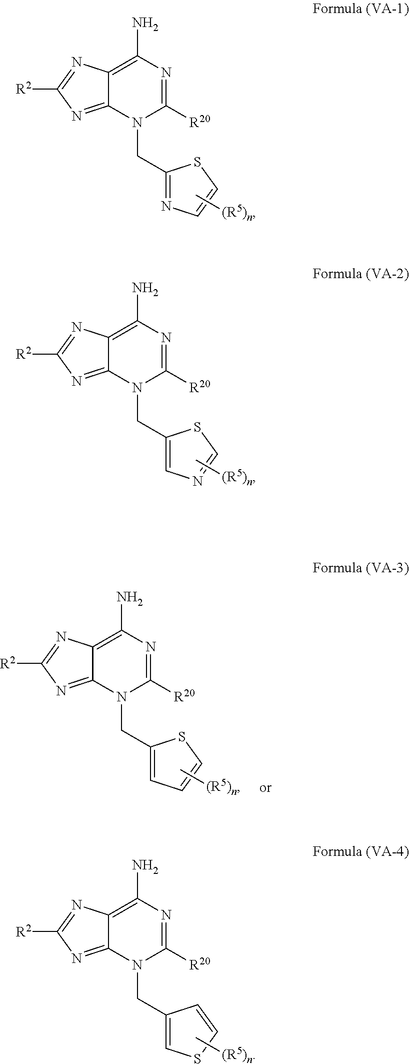

In some embodiments, the compound of Formula (I) or Formula (I-A) has the structure of Formula (VB-1) or Formula (VB-2), or a pharmaceutically acceptable salt or solvate thereof:

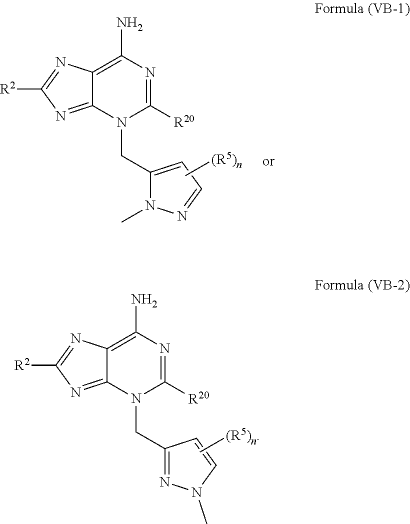

In some embodiments, R² is halogen, —CN, —OR^{a}, C₁-C₆ alkyl, or C₁-C₆ heteroalkyl; wherein alkyl and heteroalkyl is unsubstituted or substituted with one, two, or three R⁶; or R² is

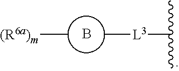

In some embodiments, R² is halogen, —CN, —OH, —OR^{a}, —SH, —SR^{a}, C₁-C₆ alkyl, C₂-C₆ alkenyl, C₂-C₆ alkynyl, or C₁-C₆ heteroalkyl; wherein alkyl, alkenyl, alkynyl and C₁-C₆ heteroalkyl is unsubstituted or substituted with one, two, or three R⁶; and each R⁶ is independently halogen, —CN, —OH, —OR^{a}, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —C(═O)N(R^{b})₂, —NR^{b}C(═O)R^{a}. In some embodiments, R² is halogen, —CN, —OR^{a}, C₁-C₆ alkyl or C₁-C₆ heteroalkyl; wherein alkyl and heteroalkyl is unsubstituted or substituted with one, two, or three R⁶; and each R⁶ is independently halogen, —OH, —OR^{a}, —N(R^{b})₂, —C(═O)R^{a}, —C(═O)OR^{b}, —OC(═O)OR^{b}, —C(═O)N(R^{b})₂, —NR^{b}C(═O)R^{a}. In some embodiments, R² is halogen, —CN, —OR^{a}, C₁-C₆ alkyl or C₁-C₆ heteroalkyl. In some embodiments, R² is —F, —Cl, —Br, or C₁-C₆ alkyl. In some embodiments, R² is —Cl, —Br, or methyl.

In some embodiments, R² is halogen, —OR^{a}, C₁-C₆ alkyl, or C₁-C₆ heteroalkyl; wherein alkyl and heteroalkyl is unsubstituted or substituted with one, two, or three R⁶; and each R⁶ is independently halogen, —OH, —OR^{a}, —SH, —SR^{a}, —S(═O)R^{a}, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}; or R² is

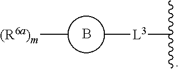

In some embodiments, R² is

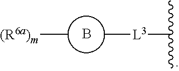

In some embodiments, Ring B is phenyl or heteroaryl. In some embodiments, Ring B is phenyl or monocyclic heteroaryl. In some embodiments, Ring B is phenyl, pyridinyl, pyrazinyl, pyrimidinyl, pyridazinyl, triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl. In some embodiments, Ring B is phenyl or a 6-membered monocyclic heteroaryl. In some embodiments, Ring B is phenyl, pyridinyl, pyrazinyl, pyrimidinyl, pyridazinyl, or triazinyl. In some embodiments, Ring B is phenyl or pyridinyl. In some embodiments, Ring B is phenyl. In some embodiments, Ring B is pyridinyl. In some embodiments, Ring B is a 5-membered monocyclic heteroaryl. In some embodiments, Ring B is furanyl, imidazolyl, pyrazolyl, oxazolyl, isoxazolyl, thiophenyl, thiazolyl, or isothiazolyl. In some embodiments, Ring B is furanyl.

In some embodiments, Ring B is cycloalkyl or heterocycloalkyl. In some embodiments, Ring B is cycloalkyl. In some embodiments, Ring B is C₃-C₈ cycloalkyl, C₃-C₆ cycloalkyl, C₃-C₅ cycloalkyl, or C₃-C₄ cycloalkyl. In some embodiments, Ring B is monocyclic cycloalkyl. In some embodiments, Ring B is cyclopropyl, cyclobutyl, cyclopentyl, cyclohexyl, cycloheptyl, or cyclooctyl. In some embodiments, Ring B is polycyclic cycloalkyl. In some embodiments, Ring B is polycyclic cycloalkyl that is spiro, fused, or bridged.

In some embodiments, Ring B is heterocycloalkyl. In some embodiments, Ring B is monocyclic heterocycloalkyl. In some embodiments, Ring B is polycyclic heterocycloalkyl. In some embodiments, Ring B is aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1 oxo thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, or 2-oxo-1,3-dioxol-4-yl.

In some embodiments, Ring B is a 6-membered aryl or heteroaryl wherein R² is:

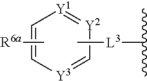

wherein: Y¹ is CH, CR^{6a}, or N; Y² is CH, CR^{6a}, or N and Y³ is CH, CR^{6a}, or N. In some embodiments, Y¹, Y², and Y³ are each N. In some embodiments, Y¹, Y², and Y³ are each CH or CR^{6a}. In some embodiments, Y¹ and Y² are CH or CR^{6a}; and Y³ is N. In some embodiments, Y¹ and Y² are N; and Y³ is CH or CR^{6a}. In some embodiments, Y¹ and Y³ are N; and Y² is CH or CR^{6a}. In some embodiments, Y¹ is CH or CR^{6a}; and Y² and Y³ are N.

In some embodiments, L³ is absent. In some embodiments, L³ is C₁-C₆ alkylene. In some embodiments, L³ is C₁-C₄ alkylene. In some embodiments, L³ is C₁-C₂ alkylene. In some embodiments, L³ is —CH₂—. In some embodiments, L³ is absent or —CH₂—.

In some embodiments, the compound of Formula (I) or Formula (I-A) has the structure of Formula (II-1), or a pharmaceutically acceptable salt or solvate thereof:

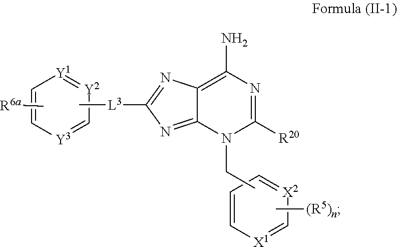

wherein:
X¹ is CH, CR⁵, or N;
X² is CH, CR⁵, or N;
Y¹ is CH, CR^{6a}, or N;
Y² is CH, CR^{6a}, or N; and
Y³ is CH, CR^{6a}, or N.

In some embodiments of a compound of Formula (II-1), is a compound of Formula (IIIA-1) or (IIIB-1); or a pharmaceutically acceptable salt thereof:

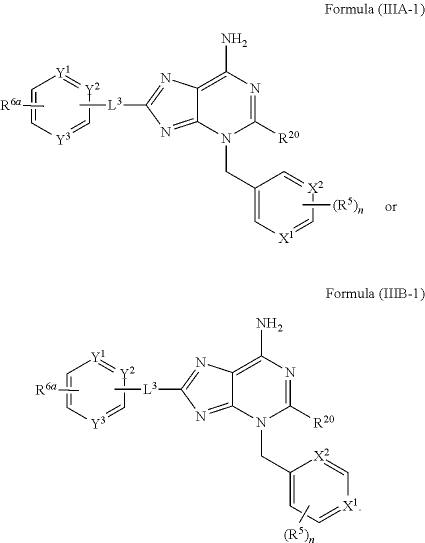

In some embodiments, the compound of Formula (I) or Formula (I-A) has the structure of Formula (V-1), or a pharmaceutically acceptable salt or solvate thereof:

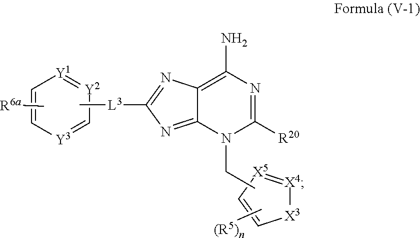

wherein:
X³ is CR⁵, CH, N, S, or O;
X⁴ is CR⁵, CH, or N;
X⁵ is CR⁵, CH, or N;
Y¹ is CH, CR^{6a}, or N;
Y² is CH, CR^{6a}, or N; and
Y³ is CH, CR^{6a}, or N.

In some embodiments, L³ is absent, or —CH₂—.

In some embodiments, each R^{6a} is independently hydrogen, halogen, —CN, —OH, —OR^{a}, —N(R^{b})₂, —C₁-C₆ alkyl, or C₁-C₆ haloalkyl.

In some embodiments, each R^{6a} is independently hydrogen, —F, —Cl, —Br, —CH₃, —NH₂, or —CF₃.

In some embodiments, when two R^{6a} groups join together with the intervening atoms of Ring B that connect the two R^{6a} groups to form a ring then the formed ring is non-aromatic or aromatic, wherein the formed ring is unsubstituted or substituted with one, two, or three halogen, —CN, —OH, C₁-C₆ alkyl, C₁-C₆ alkoxy, C₁-C₆ haloalkyl, or C₁-C₆ haloalkoxy. In some embodiments, the formed ring is fused with Ring B.

In some embodiments, when two R^{6a} groups join together with the intervening atoms of Ring B that connect the two R^{6a} groups to form a ring then the formed ring is non-aromatic, wherein the formed ring is unsubstituted or substituted with one, two, or three halogen, —CN, —OH, C₁-C₆ alkyl, C₁-C₆ alkoxy, C₁-C₆ haloalkyl, or C₁-C₆ haloalkoxy. In some embodiments, when two R^{6a} groups join together with the intervening atoms of Ring B that connect the two R^{6a} groups to form a ring then the formed ring is a non-aromatic cycloalkyl or heterocycloalkyl, wherein the formed ring is unsubstituted or substituted with one, two, or three halogen, —CN, —OH, C₁-C₆ alkyl, C₁-C₆ alkoxy, C₁-C₆ haloalkyl, or C₁-C₆ haloalkoxy.

In some embodiments, when two R^{6a} groups join together with the intervening atoms of Ring B that connect the two R^{6a} groups to form a ring then the formed ring is aromatic, wherein the formed ring is unsubstituted or substituted with one, two, or three halogen, —CN, —OH, C₁-C₆ alkyl, C₁-C₆ alkoxy, C₁-C₆ haloalkyl, or C₁-C₆ haloalkoxy.

In some embodiments, two R^{6a} groups join together with the intervening atoms of Ring B that connect the two R^{6a} groups to form a ring that is a cycloalkyl or heterocycloalkyl, wherein the formed ring is unsubstituted or substituted with one, two, or three halogen, —CN, —OH, C₁-C₆ alkyl, C₁-C₆ alkoxy, C₁-C₆ haloalkyl, or C₁-C₆ haloalkoxy. In some embodiments, two R^{6a} groups join together with the intervening atoms of Ring B that connect the two R^{6a} groups to form a ring that is a cycloalkyl, wherein the formed ring is unsubstituted or substituted with one, two, or three halogen, —CN, —OH, C₁-C₆ alkyl, C₁-C₆ alkoxy, C₁-C₆ haloalkyl or C₁-C₆ haloalkoxy. In some embodiments, two R^{6a} groups join together with the intervening atoms of Ring B that connect the two R^{6a} groups to form a ring that is a heterocycloalkyl, wherein the formed ring is unsubstituted or substituted with one, two, or three halogen, —CN, —OH, C₁-C₆ alkyl, C₁-C₆ alkoxy, C₁-C₆ haloalkyl, or C₁-C₆ haloalkoxy.

In some embodiments of a compound of Formula (I), (I-A), (V), or (V-1), is a compound of Formula (VI-A1), (VI-B1), (VI-C1), or (VI-D1); or a pharmaceutically acceptable salt thereof:

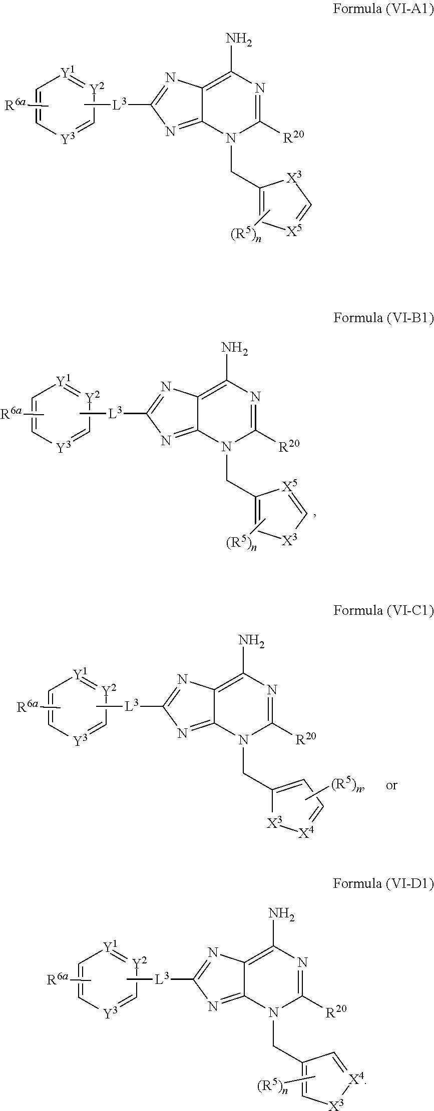

In some embodiments, of a compound of Formula (I) or Formula (I-A), or Formula (II), is a compound of Formula (VIIa) or (VIIb), or a pharmaceutically acceptable salt or solvate thereof:

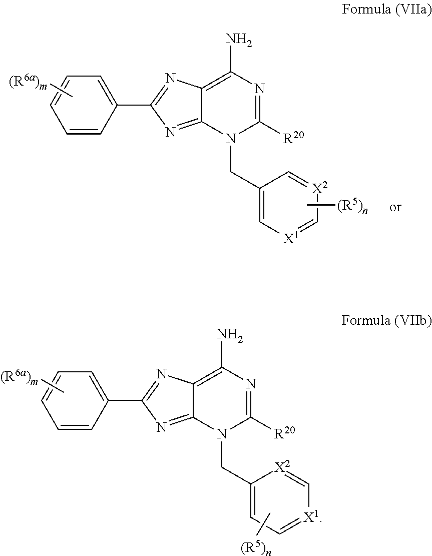

In some embodiments, of a compound of Formula (I), (I-A) or (II), is a compound of Formula (Villa) or (VIIIc); or a pharmaceutically acceptable salt or solvate thereof:

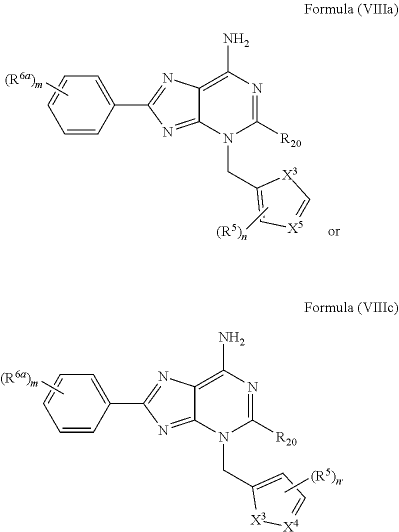

In some embodiments, each R^{6a} is independently hydrogen, halogen, —CN, —OH, —N(R^{b})₂, —C(═O)R^{a}, —C(═O)OR^{b}, —C(═O)N(R^{b})₂, C₁-C₆ alkyl, C₁-C₆ haloalkyl, or cycloalkyl which is unsubstituted or substituted with one, two, or three halogen, C₁-C₆ alkyl, or C₁-C₆ haloalkyl. In some embodiments, each R^{6a} is independently hydrogen, —F, —Cl, —Br, —CN, —OMe, —OCF₃, —OEt, —OiPr, —NH₂, —NMe₂, —C(═O)Me, —C(═O)OH, —C(═O)OMe, —C(═O)OEt, —C(═O)NH₂, —C(═O)NMe₂, —CH₃, —CH₂CH₃, —CH₂CH₂CH₃, —CH(CH₃)₂, —CF₃, —CHF₂, or —CH₂F. In some embodiments, each R^{6a} is independently hydrogen, —F, —Cl, —CN, —OMe, —OCF₃, —CH₃, or —CF₃. In some embodiments, each R^{6a} is independently hydrogen, —F, —Cl, —CN, —OMe, or —CH₃. In some embodiments, each R^{6a} is independently hydrogen, or —CH₃.

In some embodiments, two R^{6a} groups join together with the intervening atoms to form an unsubstituted or substituted cycloalkyl or unsubstituted or substituted heterocycloalkyl. In some embodiments, two R^{6a} groups join together with the intervening atoms to form an unsubstituted or substituted cycloalkyl or unsubstituted or substituted heterocycloalkyl.

In some embodiments, two R^{6a} groups join together with the intervening atoms to form an unsubstituted or substituted cycloalkyl.

In some embodiments, two R^{6a} groups join together with the intervening atoms to form an unsubstituted or substituted heterocycloalkyl. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring.

In some embodiments, m is 0. In some embodiments, m is 1. In some embodiments, m is 2. In some embodiments, m is 3. In some embodiments, m is 4. In some embodiments, m is 5. In some embodiments, m is 0 to 1, 0 to 2, 0 to 3, 0 to 4, 0 to 5, 1 to 2, 1 to 3, 1 to 4, 1 to 5, 2 to 3, 2 to 4, 2 to 5, 3 to 4, 3 to 5, or 4 to 5. In some embodiments, m is 0, 1, 2, 3, 4, or 5.

In some embodiments,

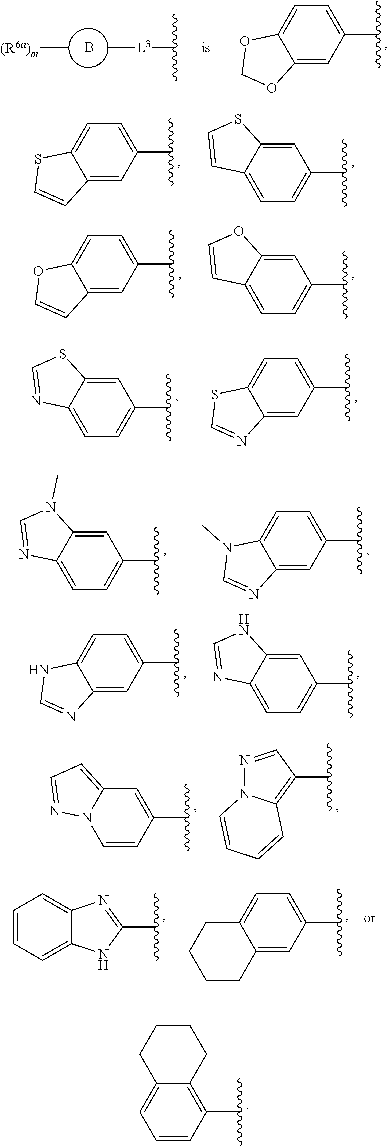

In some embodiments,

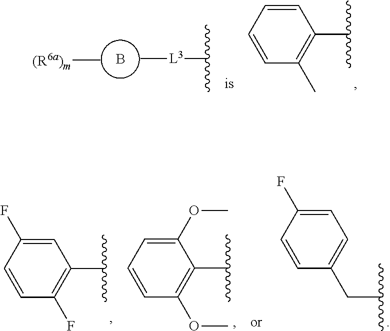

In some embodiments,

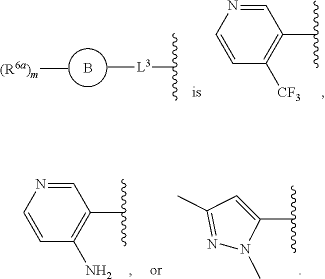

In some embodiments,

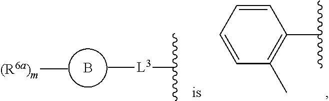

In some embodiments,

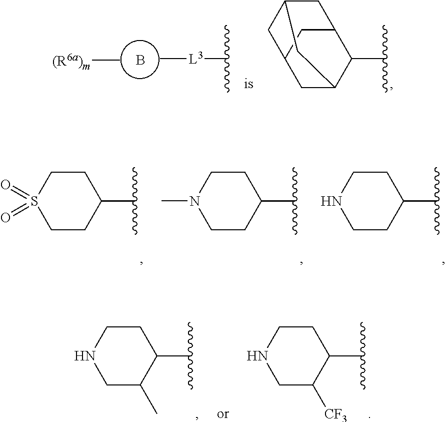

In some embodiments,

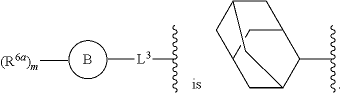

In some embodiments, each R⁵ is independently hydrogen, halogen, —CN, —OH, —OR^{a}, —N(R^{b})₂, —C(═O)R^{a}, —C(═O)OR^{b}, —C(═O)N(R^{b})₂, C₁-C₆ alkyl, C₁-C₆ haloalkyl, or cycloalkyl which is unsubstituted or substituted with one, two, or three halogen, C₁-C₆ alkyl, or C₁-C₆ haloalkyl. In some embodiments, each R⁵ is independently hydrogen, —F, —Cl, —Br, —CN, —OMe, —OCF₃, —OEt, —OiPr, —NH₂, —NMe₂, —C(═O)Me, —C(═O)OH, —C(═O)OMe, —C(═O)OEt, —C(═O)NH₂, —C(═O)NMe₂, —CH₃, —CH₂CH₃, —CH₂CH₂CH₃, —CH(CH₃)₂, —CF₃, —CHF₂, or —CH₂F. In some embodiments, each R⁵ is independently hydrogen, —F, —Cl, —Br, —CN, —OMe, —CH₃, or —CF₃. In some embodiments, each R⁵ is independently hydrogen, or —CH₃. In some embodiments, each R⁵ is independently hydrogen, —F, or —Cl. In some embodiments, each R⁵ is independently hydrogen, or —F. In some embodiments, each R⁵ is independently hydrogen, or —Cl.

In some embodiments, each R⁵ is independently hydrogen, —F, —Cl, —Br, —I, —CN, —OMe, —OCF₃, —OEt, —OiPr, —NH₂, —NMe₂, —C(═O)Me, —C(═O)OH, —C(═O)OMe, —C(═O)OEt, —C(═O)NH₂, —C(═O)NMe₂, —CH₃, —CH₂CH₃, —CH₂CH₂CH₃, —CH(CH₃)₂, or —CF₃. In some embodiments, each R⁵ is independently hydrogen, —F, —Cl, —Br, —I, —OMe, —CH₃, or —CF₃. In some embodiments, each R⁵ is independently hydrogen, —Br or

In some embodiments, each R⁵ is independently hydrogen, halogen, —CN, —OH, —OR^{a}, —N(R^{b})₂.

In some embodiments, n is 0. In some embodiments, n is 1. In some embodiments, n is 2. In some embodiments, n is 3. In some embodiments, n is 4. In some embodiments, n is 5. In some embodiments, n is 0 to 5. In some embodiments, n is 1 to 3. In some embodiments, n is 0 to 1, 0 to 2, 0 to 3, 0 to 4, 0 to 5, 1 to 2, 1 to 3, 1 to 4, 1 to 5, 2 to 3, 2 to 4, 2 to 5, 3 to 4, 3 to 5, or 4 to 5. In some embodiments, n is 0, 1, 2, 3, 4, or 5.

In some embodiments, n is 1 to 3; and each R⁵ is independently hydrogen, —F or —Cl. In some embodiments, n is 2; and each R⁵ is independently hydrogen, or

In some embodiments, each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl. In some embodiments, each R^{a} is independently C₁-C₆ alkyl, C₁-C₆ heteroalkyl, cycloalkyl, heterocycloalkyl, aryl, or heteroaryl; wherein the alkyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl. In some embodiments each R^{a} is independently C₁-C₆ alkyl or C₁-C₆ heteroalkyl; wherein the alkyl or heteroalkyl is independently unsubstituted or substituted with one, two, or three halogen, —OH, C₁-C₆ alkyl, or C₁-C₆ haloalkyl. In some embodiments, each R^{a} is independently C₁-C₆ alkyl; wherein the alkyl is unsubstituted or substituted with one, two, or three halogen.

In some embodiments, each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl. In some embodiments, each R^{b} is independently hydrogen, C₁-C₆ alkyl, C₁-C₆ heteroalkyl, cycloalkyl, heterocycloalkyl, aryl, or heteroaryl; wherein the alkyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl. In some embodiments, each R^{b} is independently hydrogen, C₁-C₆ alkyl or C₁-C₆ heteroalkyl; wherein the alkyl or heteroalkyl is independently unsubstituted or substituted with one, two, or three halogen, —OH, C₁-C₆ alkyl, or C₁-C₆ haloalkyl. In some embodiments, each R^{b} is independently C₁-C₆ alkyl; wherein the alkyl is unsubstituted or substituted with one, two, or three halogen. In some embodiments, each R^{b} is hydrogen.

In some embodiments, two R^{b} groups on a nitrogen atom are taken together with the nitrogen atom to which they are attached to form a heterocycloalkyl which is unsubstituted or substituted with one, two, or three halogen, C₁-C₆ alkyl, or C₁-C₆ haloalkyl. In some embodiments, two R^{b} groups on a nitrogen atom are taken together with the nitrogen atom to which they are attached to form a 3- to 7-membered heterocycloalkyl which is unsubstituted or substituted with one, two, or three halogen, C₁-C₆ alkyl, or C₁-C₆ haloalkyl. In some embodiments, two R^{b} groups on a nitrogen atom are taken together with the nitrogen atom to which they are attached to form a 5- or 6-membered heterocycloalkyl which is unsubstituted or substituted with one, two, or three halogen, C₁-C₆ alkyl, or C₁-C₆ haloalkyl. In some embodiments, two R^{b} groups on a nitrogen atom are taken together with the nitrogen atom to which they are attached to form pyrrolidine, piperidine, or morpholine which is unsubstituted or substituted with one, two, or three halogen, C₁-C₆ alkyl, or C₁-C₆ haloalkyl. In some embodiments, two R^{b} groups on a nitrogen atom are taken together with the nitrogen atom to which they are attached to form pyrrolidine, piperidine, or morpholine.

Any combination of the groups described above for the various variables is contemplated herein. Throughout the specification, groups and substituents thereof are chosen by one skilled in the field to provide stable moieties and compounds.

In some embodiments, compounds of Formula (I) include, but are not limited to:
3-[(3,5-dichloro(4-pyridyl))methyl]-8-(1,3-dimethylpyrazol-5-yl)-3-hydropurine-6-ylamine;
8-benzimidazol-2-yl-3-[(3,5-dichloro(4-pyridyl))methyl]-3-hydropurine-6-ylamine;
3-[(3,5-dichloro(4-pyridyl))methyl]-8-(2,5-difluorophenyl)-3-hydropurine-6-ylamine;
3-[(3,5-dichloro(4-pyridyl))methyl]-8-[4-(trifluoromethyl)(3-pyridyl)]-3-hydropurine-6-ylamine;
3-[(2,4-dichloro(3-pyridyl))methyl]-8-phenyl-3-hydropurine-6-ylamine;
({6-amino-3-[(3,5-dichloro(4-pyridyl))methyl](3-hydropurin-8-yl)}methyl)diethylamine;
8-(4-amino(3-pyridyl))-3-[(3,5-dichloro(4-pyridyl))methyl]-3-hydropurine-6-ylamine;
3-[(3,5-dichloro(4-pyridyl))methyl]-8(2-5,6,7,8-tetrahydronaphthyl)-3-hydropurine-6-ylamine;
8-(2H-benzo[d]1,3-dioxolen-5-yl)-3-[(3,5-dichloro(4-pyridyl))methyl]-3-hydropurine-6-ylamine;
3-[(3,5-dichloro(4-pyridyl))methyl]-8-[(4-fluorophenyl)methyl]-3-hydropurine-6-ylamine;
8-adamantan-2-yl-3-[(3,5-dichloro(4-pyridyl))methyl]-3-hydropurine-6-ylamine;
3-[(2,4-dichloro(3-pyridyl))methyl]-8-(1,3-dimethylpyrazol-5-yl)-3-hydropurine-6-ylamine;
8-adamantan-2-yl-3-[(2,4-dichloro(3-pyridyl))methyl]-3-hydropurine-6-ylamine;
3-[(2,4-dichloro(3-pyridyl))methyl]-8(2-5,6,7,8-tetrahydronaphthyl)-3-hydropurine-6-ylamine;
3-[(2,4-dichloro(3-pyridyl))methyl]-8-[(4-fluorophenyl)methyl]-3-hydropurine-6-ylamine;
8-(2H-benzo[d]1,3-dioxolen-5-yl)-3-[(2,4-dichloro(3-pyridyl))methyl]-3-hydropurine-6-ylamine;
8-benzothiazol-6-yl-3-[(2,4-dichloro(3-pyridyl))methyl]-3-hydropurine-6-ylamine;
8-benzo[b]furan-5-yl-3-[(3,5-dichloro(4-pyridyl))methyl]-3-hydropurine-6-ylamine;
3-[(3,5-dichloro(4-pyridyl))methyl]-8-(8-hydropyrazolo[1,5-a]pyridin-3-yl)-3-hydropurine-6-ylamine;
3-[(3,5-dichloro(4-pyridyl))methyl]-8-(1-methylbenzimidazol-6-yl)-3-hydropurine-6-ylamine;
3-[(3,5-dichloro(4-pyridyl))methyl]-8-(1-methylbenzimidazol-5-yl)-3-hydropurine-6-ylamine;
8-benzo[3,4-b]furan-6-yl-3-[(3,5-dichloro(4-pyridyl))methyl]-3-hydropurine-6-ylamine;
8-benzo[b]thiophen-5-yl-3-[(3,5-dichloro(4-pyridyl))methyl]-3-hydropurine-6-ylamine;
8-benzimidazol-5-yl-3-[(3,5-dichloro(4-pyridyl))methyl]-3-hydropurine-6-ylamine;
3-[(3,5-dichloro(4-pyridyl))methyl]-8-(8-hydropyrazolo[1,5-a]pyridin-5-yl)-3-hydropurine-6-ylamine;
3-[(3,5-dibromo(4-pyridyl))methyl]-8-(2-methylphenyl)-3-hydropurine-6-ylamine;
3-[(4-chloro-2-fluoro(3-pyridyl))methyl]-8-(2-methylphenyl)-3-hydropurine-6-ylamine;
3-[(4-methyl(1,3-thiazol-5-yl))methyl]-8-(2-methylphenyl)-3-hydropurine-6-ylamine;
3-[(3-chloro(2-thienyl))methyl]-8-(2-methylphenyl)-3-hydropurine-6-ylamine;
3-[(3-chloro(4-pyridyl))methyl]-8-(2-methylphenyl)-3-hydropurine-6-ylamine;
3-[(4,6-dichloropyrimidin-5-yl)methyl]-8-(2-methylphenyl)-3-hydropurine-6-ylamine;
3-[(4-bromo-2-chloro(3-pyridyl))methyl]-8-(2-methylphenyl)-3-hydropurine-6-ylamine;
3-[(4-bromo-1-methylpyrazol-5-yl)methyl]-8-(2-methylphenyl)-3-hydropurine-6-ylamine;
3-[(2,4-dichloro(3-pyridyl))methyl]-8-(2-methylphenyl)-3-hydropurine-6-ylamine;
3-[(3,5-dibromo(4-pyridyl))methyl]-8-phenyl-3-hydropurine-6-ylamine;
3-[(4-chloro-2-fluoro(3-pyridyl))methyl]-8-phenyl-3-hydropurine-6-ylamine;
3-[(4-methyl(1,3-thiazol-5-yl))methyl]-8-phenyl-3-hydropurine-6-ylamine;
3-[(3-chloro(2-thienyl))methyl]-8-phenyl-3-hydropurine-6-ylamine;
3-[(4,6-dichloropyrimidin-5-yl)methyl]-8-phenyl-3-hydropurine-6-ylamine;
3-[(4-bromo-2-chloro(3-pyridyl))methyl]-8-phenyl-3-hydropurine-6-ylamine;
3-[(4-bromo-1-methylpyrazol-5-yl)methyl]-8-phenyl-3-hydropurine-6-ylamine;
3-[(2,4-dimethyl(1,3-thiazol-5-yl))methyl]-8-(2-methylphenyl)-3-hydropurine-6-ylamine;
3-[(3-fluoro(4-pyridyl))methyl]-8-(2-methylphenyl)-3-hydropurine-6-ylamine;
3-[(5-chloro(3-pyridyl))methyl]-8-(2-methylphenyl)-3-hydropurine-6-ylamine;
3-[(2-methyl (3-pyridyl))methyl]-8-(2-methylphenyl)-3-hydropurine-6-ylamine;
3-[(3,5-dibromo(4-pyridyl))methyl]-8-(8-hydropyrazolo[1,5-a]pyridin-3-yl)-3-hydropurine-6-ylamine;
3-[(3,5-dibromo(4-pyridyl))methyl]-8-(2-5,6,7,8-tetrahydronaphthyl)-3-hydropurine-6-ylamine;
3-[(3,5-dibromo(4-pyridyl))methyl]-2-methyl-8-(2-methylphenyl)-3-hydropurine-6-ylamine;
3-[(3,5-dichloro(4-pyridyl))methyl]-2-methyl-8-(2-methylphenyl)-3-hydropurine-6-ylamine;
3-[(4-bromo-1-methylpyrazol-5-yl)methyl]-2-methyl-8-(2-methylphenyl)-3-hydropurine-6-ylamine;
3-[(2,4-dichloro(3-pyridyl))methyl]-2-methyl-8-(2-methylphenyl)-3-hydropurine-6-ylamine;
3-[(3,5-dibromo(4-pyridyl))methyl]-8-(5,6,7,8-tetrahydronaphthyl)-3-hydropurine-6-ylamine;
3-[(3,5-dichloro(4-pyridyl))methyl]-8-(5,6,7,8-tetrahydronaphthyl)-3-hydropurine-6-ylamine;
3-[(3,5-diiodo(4-pyridyl))methyl]-8-(2-methylphenyl)-3-hydropurine-6-ylamine;
3-[(4-iodo-2-methoxy(3-pyridyl))methyl]-8-(2-methylphenyl)-3-hydropurine-6-ylamine;
8-(2-methylphenyl)-3-[(2,3,5-trichloro(4-pyridyl))methyl]-3-hydropurine-6-ylamine;
3-[(4-iodo-2-methoxy(3-pyridyl))methyl]-8-phenyl-3-hydropurine-6-ylamine;
8-(2,6-dimethoxyphenyl)-3-[(2,3,5-trichloro(4-pyridyl))methyl]-3-hydropurine-6-ylamine;
3-[(2,4-dimethyl(3-pyridyl))methyl]-8-(2-methylphenyl)-3-hydropurine-6-ylamine;
3-[(2,4-dimethyl(3-pyridyl))methyl]-8-phenyl-3-hydropurine-6-ylamine;
3-[(2,4-dimethyl(3-pyridyl))methyl]-8-phenyl-3-hydropurine-6-ylamine;
3-[(3,5-dimethoxy(4-pyridyl))methyl]-8-(2-methylphenyl)-3-hydropurine-6-ylamine;
3-[(2-amino-4,6-dichloropyrimidin-5-yl)methyl]-8-(2-methylphenyl)-3-hydropurine-6-ylamine;
8-(2,6-dimethoxyphenyl)-3-[(2,4,5-trichloro(3-pyridyl))methyl]-3-hydropurine-6-ylamine; or
3-[(2,4-dimethoxy(3-pyridyl))methyl]-8-(2-methylphenyl)-3-hydropurine-6-ylamine;
   or a pharmaceutically acceptable salt or solvate thereof.

Any combination of the groups described above for the various variables is contemplated herein. Throughout the specification, groups and substituents thereof are chosen by one skilled in the field to provide stable moieties and compounds.

### Further Forms of Compounds

In some aspects, a compound disclosed herein possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In one aspect, prodrugs are designed to alter the metabolic stability or the transport characteristics of a drug, to mask side effects or toxicity, to improve the flavor of a drug or to alter other characteristics or properties of a drug. By virtue of knowledge of pharmacokinetic, pharmacodynamic processes and drug metabolism in vivo, once a pharmaceutically active compound is known, the design of prodrugs of the compound is possible. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Rooseboom et al., Pharmacological Reviews, 56:53-102, 2004; Aesop Cho, “Recent Advances in Oral Prodrug Discovery”, Annual Reports in Medicinal Chemistry, Vol. 41, 395-407, 2006; T. Higuchi and V. Stella, Pro-drugs as Novel Delivery Systems, Vol. 14 of the A.C.S. Symposium Series).

In some embodiments, some of the herein-described compounds may be a prodrug for another derivative or active compound.

In some embodiments, sites on the aromatic ring portion of compounds described herein are susceptible to various metabolic reactions Therefore incorporation of appropriate substituents on the aromatic ring structures will reduce, minimize or eliminate this metabolic pathway. In specific embodiments, the appropriate substituent to decrease or eliminate the susceptibility of the aromatic ring to metabolic reactions is, by way of example only, a halogen, or an alkyl group.

In another embodiment, the compounds described herein are labeled isotopically (e.g., with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine, chlorine, and iodine such as, for example, ²H, ³H, ¹³C, ¹⁴C, ¹⁵N, ¹⁸O, ¹⁷O, ³⁵S, ¹⁸F, ³⁶Cl, and ¹²⁵I. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

“Pharmaceutically acceptable” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound disclosed herein with acids. Pharmaceutically acceptable salts are also obtained by reacting a compound disclosed herein with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g., lithium, sodium, potassium), an alkaline earth ion (e.g., magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Methods of Synthesis

In some embodiments, the syntheses of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure).

In one aspect, the compounds described herein are synthesized as exemplified in Scheme 1.

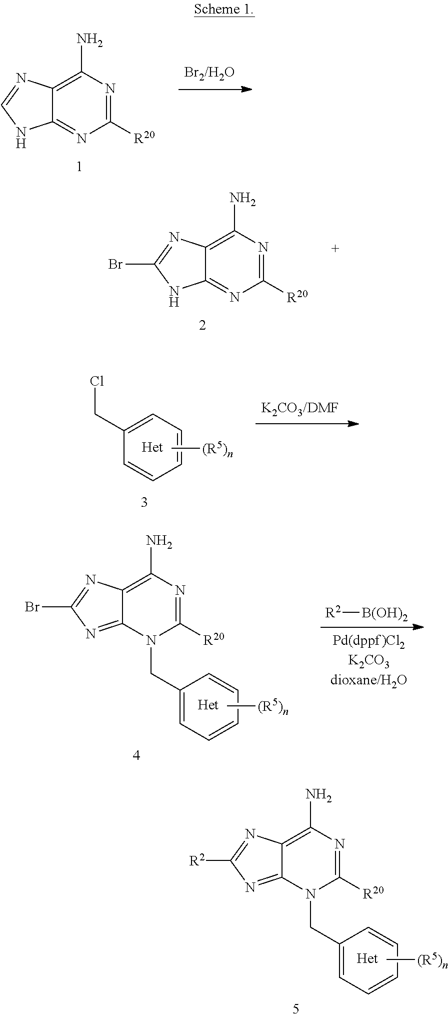

In some embodiments, a 9H-purin-6-amine derivative 1 is reacted with bromine to give compound 2. In some embodiments, the 9H-purin-6-amine derivative is 9H-purin-6-amine (i.e., R²⁰ is H). In some embodiments, compound 2 is reacted with heteroarylmethyl chlorides 3 and potassium carbonate to give compound 4. In some embodiments, compound 4 is coupled with a suitable boronic acid (R²—B(OH)₂) using a suitable palladium catalyst to give compound 5.

In another aspect, the compounds described herein are synthesized as exemplified in Scheme 2.

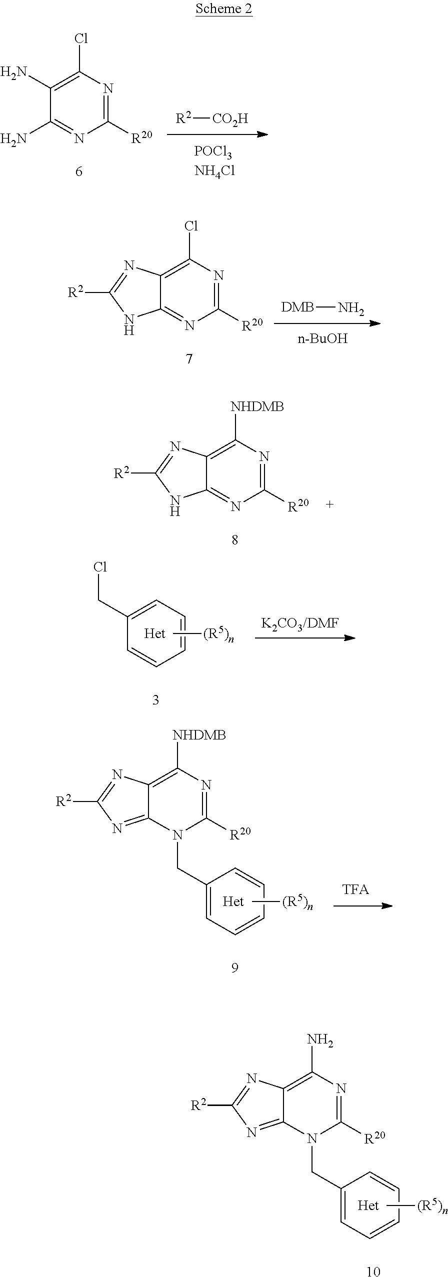

In some embodiments, a 6-chloropyrimidine-4,5-diamine derivative 6 is condensed with a carboxylic acid to give purine derivative 7. In some embodiments, the 6-chloropyrimidine-4,5-diamine derivative 6 is 6-chloropyrimidine-4,5-diamine (i.e., R²⁰ is H). In some embodiments, compound 7 is reacted with dimethoxybenzylamine to give compound 8. In some embodiments, compound 8 is reacted with heteroarylmethyl chlorides 3 under basic conditions to give compound 9. In some embodiments, compound 9 is then deprotected using TFA to give desired compound 10.

In some embodiments, the —NH₂ group of the compounds depicted in Scheme 1 or Scheme 2 is further modified. For example, in some embodiment the —NH₂ group is reacted with, but not limited to, an alkylating reagent such as an alkyl halide. In other embodiments, the —NH₂ group is reacted with, but not limited to, a carboxylic acid and coupling reagent such as EDC, DCC, BOP, HATU or the like, or a carboxylic acid activated ester or an acid halide, alkylchloroformate, arylchloroformate, benzylchloroformate, alkylisocyanate, benzylisocyanate, arylisocyanate, alkyl sulfonyl chloride, arylsulfonyl chloride, heteroarylsulfonyl chloride, or the like.

It is understood that other analogous procedures and reagents could be used, and that these Schemes are only meant as non-limiting examples.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed invention.

The terms below, as used herein, have the following meanings, unless indicated otherwise:

“Oxo” refers to the ═O substituent.

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. Unless stated otherwise specifically in the specification, an alkyl group may be optionally substituted as described below.

“Alkylene” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

“Heteroalkyl” refers to an alkyl radical as described above where one or more carbon atoms of the alkyl is replaced with a O, N (i.e., NH, N-alkyl) or S atom. “Heteroalkylene” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Alkylamino” refers to a radical of the formula —NHR or —NRR where each R is, independently, an alkyl radical as defined above. Unless stated otherwise specifically in the specification, an alkylamino group may be optionally substituted as described below.

The term “aromatic” refers to a planar ring having a delocalized π-electron system containing 4n+2π electrons, where n is an integer. Aromatics can be optionally substituted. The term “aromatic” includes both aryl groups (e.g., phenyl, naphthalenyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to:

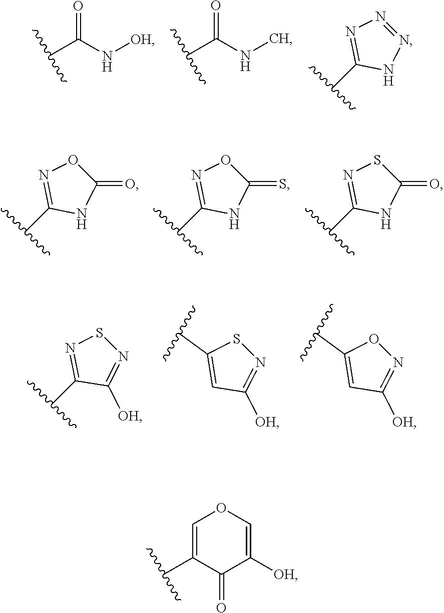

and the like.

“Cycloalkyl” refers to a monocyclic or polycyclic non-aromatic radical, wherein each of the atoms forming the ring (i.e., skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. In some embodiments, a cycloalkyl is a C₃-C₆cycloalkyl. In some embodiments, the cycloalkyl is monocyclic, bicyclic or polycyclic. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, bicyclo[1.1.1]pentyl, bicyclo[3.3.0]octane, bicyclo[4.3.0]nonane, bicyclo[2.1.1]hexane, bicyclo[2.2.1]heptane, bicyclo[2.2.2]octane, bicyclo[3.2.2]nonane, bicyclo[3.3.2]decane, norbornyl, decalinyl and adamantyl. In some embodiments, the cycloalkyl is monocyclic. Monocyclic cycicoalkyl radicals include, for example, cyclopropyl, cyclobutyl, cyclopentyl, cyclohexyl, cycloheptyl, and cyclooctyl. In some embodiments, the monocyclic cycicoalkyl is cyclopropyl, cyclobutyl, cyclopentyl or cyclohexyl. In some embodiments, the cycloalkyl is bicyclic. Bicyclic cycloalkyl groups include fused bicyclic cycloalkyl groups, spiro bicyclic cycloalkyl groups, and bridged bicyclic cycloalkyl groups. In some embodiments, cycloalkyl groups are selected from among spiro[2.2]pentyl, bicyclo[1.1.1]pentyl, bicyclo[3.3.0]octane, bicyclo[4.3.0]nonane, bicyclo[2.1.1]hexane, bicyclo[2.2.1]heptane, bicyclo[2.2.2]octane, bicyclo[3.2.2]nonane, bicyclo[3.3.2]decane, norbornyl, 3,4-dihydronaphthalen-1(2H)-one and decalinyl. In some embodiments, the cycloalkyl is polycyclic. Polycyclic radicals include, for example, adamantyl, and. In some embodiments, the polycyclic cycloalkyl is adamantyl. Unless otherwise stated specifically in the specification, a cycloalkyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heterocycloalkyl” or “heterocyclyl” or “heterocyclic ring” refers to a stable 3- to 14-membered non-aromatic ring radical comprising 2 to 10 carbon atoms and from one to 4 heteroatoms selected from the group consisting of nitrogen, oxygen, and sulfur. Unless stated otherwise specifically in the specification, the heterocycloalkyl radical may be a monocyclic, bicyclic ring (which may include a fused bicyclic heterocycloalkyl (when fused with an aryl or a heteroaryl ring, the heterocycloalkyl is bonded through a non-aromatic ring atom), bridged heterocycloalkyl or spiro heterocycloalkyl), or polycyclic. In some embodiments, the heterocycloalkyl is monocyclic or bicyclic. In some embodiments, the heterocycloalkyl is monocyclic. In some embodiments, the heterocycloalkyl is bicyclic. The nitrogen, carbon or sulfur atoms in the heterocyclyl radical may be optionally oxidized. The nitrogen atom may be optionally quaternized. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring. In some embodiments, heterocycloalkyls have from 2 to 10 carbons, 1-2 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl group may be optionally substituted.

“Heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, a bicyclic heteroaryl is a C₆-C₉heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halogen, acyl, acyloxy, —CO₂H, —CO₂alkyl, nitro, and amino, including mono- and di-substituted amino groups (e.g., —NH₂, —NHR, —NR₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

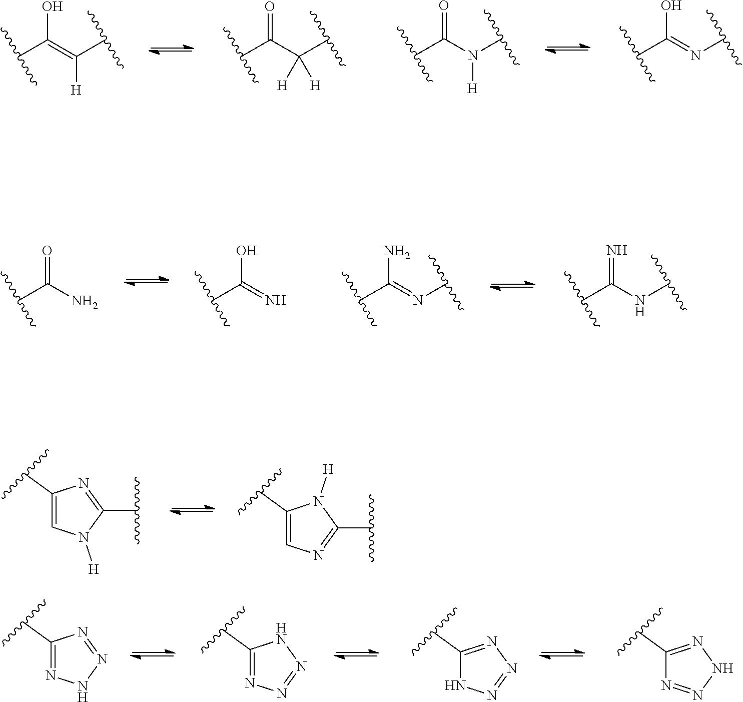

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmaceutical combination” as used herein, means a product that results from the mixing or combining of more than one active ingredient and includes both fixed and non-fixed combinations of the active ingredients. The term “fixed combination” means that the active ingredients, e.g., a compound of Formula (I) and a co-agent, are both administered to a patient simultaneously in the form of a single entity or dosage. The term “non-fixed combination” means that the active ingredients, e.g., a compound of Formula (I) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g., the administration of three or more active ingredients.

The term “subject” or “patient” encompasses mammals. Examples of mammals include, but are not limited to, humans. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In one aspect, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound disclosed herein with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Pharmaceutical formulations described herein are administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds disclosed herein are administered orally.

In some embodiments, the compounds disclosed herein are administered topically. In such embodiments, the compound disclosed herein is formulated into a variety of topically administrable compositions, such as solutions, suspensions, lotions, gels, pastes, shampoos, scrubs, rubs, smears, medicated sticks, medicated bandages, balms, creams or ointments. In one aspect, the compounds disclosed herein are administered topically to the skin.

In another aspect, the compounds disclosed herein are administered by inhalation.

In another aspect, the compounds disclosed herein are formulated for intranasal administration. Such formulations include nasal sprays, nasal mists, and the like.

In another aspect, the compounds disclosed herein are formulated as eye drops.

In any of the aforementioned aspects are further embodiments in which the effective amount of the compound disclosed herein is: (a) systemically administered to the mammal; and/or (b) administered orally to the mammal; and/or (c) intravenously administered to the mammal; and/or (d) administered by inhalation to the mammal; and/or (e) administered by nasal administration to the mammal; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of the compound disclosed herein, including further embodiments in which (i) the compound is administered once; (ii) the compound is administered to the mammal multiple times over the span of one day; (iii) the compound is administered continually; or (iv) the compound is administered continuously.

In any of the aforementioned aspects are further embodiments comprising multiple administrations of the effective amount of the compound disclosed herein, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound disclosed herein is temporarily suspended or the dose of the compound being administered is temporarily reduced; at the end of the drug holiday, dosing of the compound is resumed. In one embodiment, the length of the drug holiday varies from 2 days to 1 year.

In certain embodiments, the compound disclosed herein is administered in a local rather than systemic manner.

In some embodiments, the compound disclosed herein is administered topically. In some embodiments, the compound disclosed herein is administered systemically.

In some embodiments, the pharmaceutical formulation is in the form of a tablet. In other embodiments, pharmaceutical formulations of the compounds disclosed herein are in the form of a capsule.

In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a compound disclosed herein is formulated for use as an aerosol, a mist or a powder.

For buccal or sublingual administration, the compositions may take the form of tablets, lozenges, or gels formulated in a conventional manner.

In some embodiments, compounds disclosed herein are prepared as transdermal dosage forms.

In one aspect, a compound disclosed herein is formulated into a pharmaceutical composition suitable for intramuscular, subcutaneous, or intravenous injection.

In some embodiments, the compound disclosed herein is be administered topically and can be formulated into a variety of topically administrable compositions, such as solutions, suspensions, lotions, gels, pastes, medicated sticks, balms, creams or ointments.

In some embodiments, the compounds disclosed herein are formulated in rectal compositions such as enemas, rectal gels, rectal foams, rectal aerosols, suppositories, jelly suppositories, or retention enemas.

### Methods of Dosing and Treatment Regimens

In one aspect, the compounds disclosed herein are used in the preparation of medicaments for the treatment of diseases or conditions described herein. In addition, a method for treating any of the diseases or conditions described herein in a subject in need of such treatment, involves administration of pharmaceutical compositions that include at least one compound disclosed herein or a pharmaceutically acceptable salt, active metabolite, prodrug, or solvate thereof, in therapeutically effective amounts to said subject.

In certain embodiments, the compositions containing the compound disclosed herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds disclosed herein are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

Doses employed for adult human treatment are typically in the range of 0.01 mg-5000 mg per day or from about 1 mg to about 1000 mg per day. In one embodiment, the desired dose is conveniently presented in a single dose or in divided doses.

### Combination Treatments

In certain instances, it is appropriate to administer at least one compound disclosed herein in combination with another therapeutic agent.

In one specific embodiment, a compound disclosed herein is co-administered with a second therapeutic agent, wherein the compound disclosed herein and the second therapeutic agent modulate different aspects of the disease, disorder or condition being treated, thereby providing a greater overall benefit than administration of either therapeutic agent alone.

For combination therapies described herein, dosages of the co-administered compounds vary depending on the type of co-drug(s) employed, on the specific drug(s) employed, on the disease or condition being treated and so forth. In additional embodiments, when co-administered with one or more other therapeutic agents, the compound provided herein is administered either simultaneously with the one or more other therapeutic agents, or sequentially.

If administration is simultaneous, the multiple therapeutic agents are, by way of example only, provided in a single, unified form, or in multiple forms.

In some embodiments, the one or more agents used in the treatment of a metabolic disorder include, but are not limited to, a statin, an insulin sensitizing drug, (such as sitagliptin, vildagliptin, saxagliptin, linagliptin, anaglptin, teneligliptin, alogliptin, gemiglptin, or dutoglpitin), meglitinide, sulfonylurea, peroxisome proliferator-activated receptor (alpha-glucosidase inhibitor, amylin agonist, dipeptidyl-peptidase 4 (DPP-4) inhibitor PPAR)-gamma agonist (e.g., a thiazolidinedione (TZD) [such as ioglitazone, rosiglitazone, rivoglitazone, or troglitazone], aleglitazar, farglitazar, muraglitazar, or tesaglitazar), a glucagon-like peptide (GLP) agonist, anti-inflammatory agent (e.g., oral corticosteroid), or a combination thereof. In some embodiments, the one or more agents used in the treatment of a metabolic disorder include, but are not limited to, a statin, HMG-CoA reductase inhibitor, fish oil, fibrate, niacin or other treatment for dyslipidemia. In some embodiments retinoic acid is also administered. In one example, nicotinamide ribonucleoside and/or nicotinamide ribonucleoside analogs are also administered.

In some embodiments, the additional therapeutic agent is a peroxisome proliferator activated receptor (PPAR) agonist (gamma, dual, or pan), a dipeptidyl peptidase (IV) inhibitor, a glucagon-like peptide-1 (GLP-I) analog, insulin or an insulin analog, an insulin secretagogue, a sodium glucose co-transporter 2 (SGLT2) inhibitor, a human amylin analog, a biguanide, a glucophage, an alpha-glucosidase inhibitor, a meglitinide, a thiazolidinedione, a sulfonylurea, or any combination thereof.

In some embodiments, the additional therapeutic agent is an angiotensin-converting enzyme (ACE) inhibitor, angiotensin II receptor blocker (ARB), beta-blocker, diuretic, calcium channel blocker, inhibitor of renin-angiotensin system (RAS), blood-thinning medication, a statin, a fibrate, or any combination thereof.

### Methods of Inhibition

In one aspect, described herein is a method of inhibiting low molecular weight protein tyrosine phosphatase (LMPTP) activity comprising contacting the low molecular weight protein tyrosine phosphatase (LMPTP) with a compound disclosed herein, or a pharmaceutically acceptable salt or solvate thereof. In another aspect is a method of inhibiting low molecular weight protein tyrosine phosphatase (LMPTP) activity in a mammal comprising administering to the mammal a compound disclosed herein, or a pharmaceutically acceptable salt or solvate thereof.

In some embodiments, the mammal has type 2 diabetes, cardiovascular disease, coronary artery disease, hyperlipidemia, lipodystrophy, insulin resistance, rheumatic disease, atherosclerosis, myocardial infarction, stroke, high blood pressure (hypertension), obesity, elevated fasting plasma glucose, high serum triglycerides, elevated blood cholesterol, heart failure, metabolic syndrome, or a combination thereof. In some embodiments, the mammal has insulin resistance, metabolic syndrome, type 2 diabetes, cardiovascular disease, or a combination thereof. In some embodiments, the mammal has insulin resistance. In some embodiments, the mammal has metabolic syndrome. In some embodiments, the mammal has type 2 diabetes. In some embodiments, the mammal has cardiovascular disease. In some embodiments, the mammal has an impaired glucose tolerance. In some embodiments, the mammal is pre-diabetic. In some embodiments, the mammal is obese. In come embodiments, the compound modulates glucose and lipid metabolism.

In some embodiments, the mammal has a disease or condition that would benefit from inhibition of LMPTP activity. In some embodiments, the disease or condition is described herein.

In some embodiments, the method includes administering a second agent (e.g., therapeutic agent). In some embodiments, the method includes administering a second agent (e.g., therapeutic agent) in a therapeutically effective amount. Examples of a second agent include therapeutic agents known in the art for the treatment of diabetes, heart disease, coronary artery disease, hyperlipidemia, lipodystrophy, insulin resistance, rheumatic disease, atherosclerosis, myocardial infarction, stroke, high blood pressure (hypertension), obesity, elevated fasting plasma glucose, high serum triglycerides, elevated blood cholesterol, cardiac hypertrophy, heart failure (e.g., hypertrophy-induced heart failure) or metabolic syndrome. Thus, in some embodiments, the method includes administering to a subject in need thereof an effective amount of a compound described herein in combination with a second therapeutic agent for the treatment of diabetes, heart disease, coronary artery disease, hyperlipidemia, lipodystrophy, insulin resistance, rheumatic disease, atherosclerosis, myocardial infarction, stroke, high blood pressure (hypertension), obesity, elevated fasting plasma glucose, high serum triglycerides, elevated blood cholesterol, cardiac hypertrophy, heart failure (e.g., hypertrophy-induced heart failure) or metabolic syndrome.

### Methods of Treatment

In one aspect, described herein is a method of treating a disease or condition including administering to a subject in need thereof an effective amount of a compound disclosed herein. In one aspect, the disease or condition being treated is a metabolic disease or condition.

In some embodiments, the disease or condition is type 2 diabetes, cardiovascular disease, coronary artery disease, hyperlipidemia, lipodystrophy, insulin resistance, rheumatic disease, atherosclerosis, myocardial infarction, stroke, high blood pressure (hypertension), obesity, elevated fasting plasma glucose, high serum triglycerides, elevated blood cholesterol, heart failure, metabolic syndrome, or a combination thereof. In some embodiments, the mammal has an impaired glucose tolerance. In some embodiments, the mammal is pre-diabetic. In some embodiments, the mammal is obese. In come embodiments, the compound modulates glucose and lipid metabolism.

In one aspect is provided a method of treating a disease associated with low molecular weight protein tyrosine phosphatase (LMPTP) activity including administering to a subject in need thereof an effective amount of a compound described herein. In some embodiments, the disease is associated with aberrant low molecular weight protein tyrosine phosphatase (LMPTP) activity. For example, studies have shown that inhibition of low molecular weight protein tyrosine phosphatase (LMPTP) activity may be a target for cardiac diseases (e.g., heart failure). See, e.g., Wade et al., J. Pathol., 2015, pages 1-13 (DOI: 10.1002/path.4594), which is hereby incorporated by reference in its entirety.

In some embodiments, the method includes administering a second agent (e.g., therapeutic agent). In some embodiments, the method includes administering a second agent (e.g., therapeutic agent) in a therapeutically effective amount. Examples of a second agent include therapeutic agents known in the art for the treatment of diabetes, heart disease, coronary artery disease, hyperlipidemia, lipodystrophy, insulin resistance, rheumatic disease, atherosclerosis, myocardial infarction, stroke, high blood pressure (hypertension), obesity, elevated fasting plasma glucose, high serum triglycerides, elevated blood cholesterol, cardiac hypertrophy, heart failure (e.g., hypertrophy-induced heart failure) or metabolic syndrome. Thus, in some embodiments, the method includes administering to a subject in need thereof an effective amount of a compound described herein in combination with a second therapeutic agent for the treatment of diabetes, heart disease, coronary artery disease, hyperlipidemia, lipodystrophy, insulin resistance, rheumatic disease, atherosclerosis, myocardial infarction, stroke, high blood pressure (hypertension), obesity, elevated fasting plasma glucose, high serum triglycerides, elevated blood cholesterol, cardiac hypertrophy, heart failure (e.g., hypertrophy-induced heart failure) or metabolic syndrome.

### EXAMPLES

### Preparation of Compounds

Abbreviations

| | |
|---|---|
| CAN: | Ceric ammonium nitrate |
| DCM: | Dichloromethane |
| DIEA: | Diisopropylethylamine |
| DMF: | Dimethyl formamide |
| DMSO: | Dimethyl sulfoxide |
| EA: | Ethyl acetate |
| ESI: | Electrospray ionization |
| HATU: | 1-[Bis(dimethylamino)methylene]-1H-1,2,3-triazolo[4,5-b]pyridinium 3-oxid |
| | hexafluorophosphate |
| HPLC: | High performance liquid chromatography |
| HRMS: | High resolution mass spectrometry |
| h or hr(s): | Hour(s) |
| MeOH: | Methanol |
| Ms: | Mesyl, or methanesulfonyl |
| min(s): | Minutes |
| m/z: | Mass-to-charge ratio |
| 1H NMR: | Proton nuclear magnetic resonance |
| 13C NMR: | Carbon nuclear magnetic resonance |
| PE: | Petroleum ether |
| rt: | Room temperature |
| TFA: | Trifluoroacetic acid |

### Example 1: Synthesis of 8-bromo-3-((3,5-dichloropyridin-4-yl)methyl)-3H-purin-6-amine

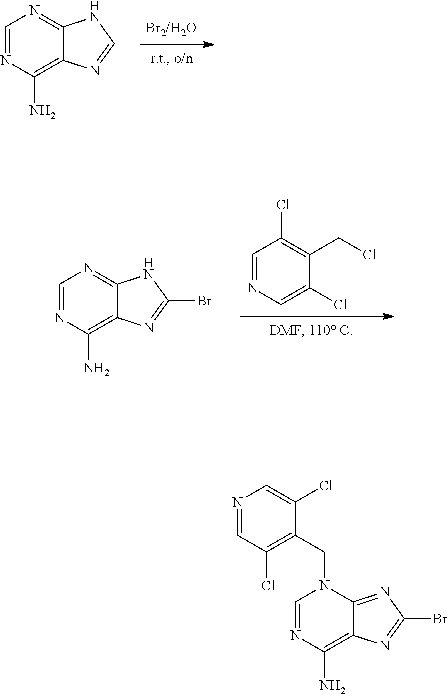 [SMILES: Nc1ncnc2[nH]cnc12 ; Nc1ncnc2[nH]c(Br)nc12 ; ClCc1c(Cl)cncc1Cl ; Nc1ncn(Cc2c(Cl)cncc2Cl)c2nc(Br)nc1-2]

### Step 1

To a solution of 9H-purin-6-amine (2.2 g, 0.016 mol) in H₂O (200 mL) was added Br₂ (6 mL). The reaction was stirred at r.t. overnight. The reaction mixture was concentrated in vacuo. The residue was washed with water (15 mL×2) and EA (15 mL×2) and dried to give 8-bromo-9H-purin-6-amine (1.8 g, yield: 52.9%) as a yellow solid.

### Step 2

To a solution of 8-bromo-9H-purin-6-amine (800 mg, 3.75 mmol) in DMF (5 mL) was added 3,5-dichloro-4-(chloromethyl)pyridine (1.6 g, 8.26 mmol). The reaction was stirred at 110° C. overnight. After cooling down to room temperature, the reaction mixture was concentrated and purified by prep-HPLC to give 8-bromo-3-((3,5-dichloropyridin-4-yl)methyl)-3H-purin-6-amine (620 mg, yield: 44.6%) as a white solid.

¹H NMR (400 MHz, DMSO-d₆): δ=8.68 (s, 2H), 8.34 (s, 2H), 814 (s, 1H), 5.72 (s, 2H); MS: m/z 372.9 (M+H)⁺.

### Example 2: Synthesis of 3-((3,5-dichloropyridin-4-yl)methyl)-8-(o-tolyl)-3H-purin-6-amine

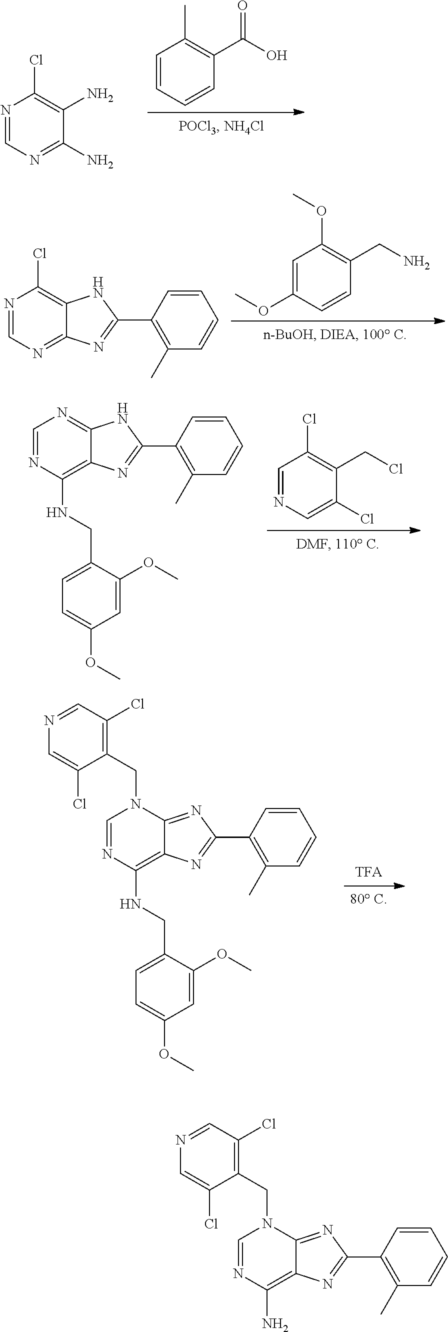

### Step 1

To a solution of 6-chloropyrimidine-4,5-diamine (500 mg, 3.47 mmol) in POCl₃ (10 mL) was added 2-methylbenzoic acid (473 g, 3.47 mmol) and NH₄C₁ (551 mg, 10.41 mmol) at room temperature. The reaction mixture was then heated to 110° C. and stirred overnight. After cooling down to room temperature, the reaction was evaporated to remove POCl₃. The residue was dissolved in water (20 mL) and neutralized with NH₃.H₂O to pH 7-8. The suspension was filtered through funnel. The filtered cake was dried to give 6-chloro-8-(o-tolyl)-7H-purine (749 mg, yield: 88.4%) as a yellow solid. MS: m/z 245.2 (M+H⁺).

### Step 2

To a solution of 6-chloro-8-(o-tolyl)-7H-purine (749 mg, 3.07 mmol) in n-BuOH (10 mL) was added dropwise DIEA (792 mg, 6.14 mmol) and 2,4-dimethoxy-benzyl amine (512 mg, 3.07 mmol) at room temperature. The reaction mixture was then heated to 100° C. and stirred overnight. After cooling down to room temperature, the reaction mixture was concentrated in vacuum. The residue was washed with EA (20 mL×3) and dried to give N-(2,4-dimethoxybenzyl)-8-(o-tolyl)-9H-purin-6-amine (522 mg, yield: 45.4%) as a yellow solid. MS: m/z 376.4 (M+H⁺).

### Step 3

To a solution of N-(2,4-dimethoxybenzyl)-8-(o-tolyl)-9H-purin-6-amine (370 mg, 0.99 mmol) in DMF (10 mL) was added 3,5-dichloro-4-(chloromethyl)pyridine (385 mg, 1.97 mmol) at room temperature. The reaction mixture was then heated to 110° C. and stirred overnight. After cooling down to room temperature and filtered. The filtered cake was washed with MeOH (10 mL×3) and dried to give 3-((3,5-dichloropyridin-4-yl)methyl)-N-(2,4-dimethoxybenzyl)-8-(o-tolyl)-3H-purin-6-amine (635 mg, yield: 100%) as a yellow solid. MS: m/z 535.3 (M+H⁺).

### Step 4

A solution of 3-((3,5-dichloropyridin-4-yl)methyl)-N-(2,4-dimethoxybenzyl)-8-(o-tolyl)-3H-purin-6-amine (500 mg, 0.94 mmol) in TFA (10 mL) was stirred at 80° C. for 4 hrs. After cooling down to room temperature, the reaction mixture was concentrated in vacuum. The residue was washed with NaHCO₃ aqueous (10 mL×3) and DCM (10 mL×3) and dried to give 3-((3,5-dichloropyridin-4-yl)methyl)-8-(o-tolyl)-3H-purin-6-amine (162 mg, yield: 46.4%) as a gray solid.

¹H NMR (400 MHz, DMSO-d₆): δ=8.65 (s, 2H), 8.52 (s, 1H), 8.04-8.01 (m, 3H), 7.20-7.17 (m, 3H), 5.81 (s, 2H), 3.36 (s, 3H). MS: m/z 385.0 (M+H⁺).

The following compound was prepared using analogous procedures to Example 2.

| Name | Structure | Characterization |
|---|---|---|
| 4-(6-amino-3-((3,5- dichloropyridin-4- yl)methyl)-3H-purin-8- yl)tetrahydro-2H- thiopyran 1,1-dioxide | 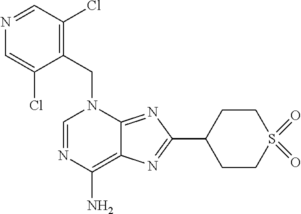 [SMILES: Nc1ncn(Cc2c(Cl)cncc2Cl)c2nc(C3CCS(=O)(=O)CC3)nc1-2] | ¹H NMR (400 MHz, DMSO-d₆): δ = 8.64 (s, 2H), 8.38 (s, 1H), 7.87 (brs, 2H), 5.73 (s, 2H), 3.12-3.06 (m, 5H), 2.24-2.18 (m, 4H). MS: m/z 427.0 (M + H⁺). |

### Example 3: Synthesis of 3-((3,5-dichloropyridin-4-yl)methyl)-8-(piperidin-4-yl)-3H-purin-6-amine and 3-((3,5-dichloropyridin-4-yl)methyl)-8-(1-methylpiperidin-4-yl)-3H-purin-6-amine

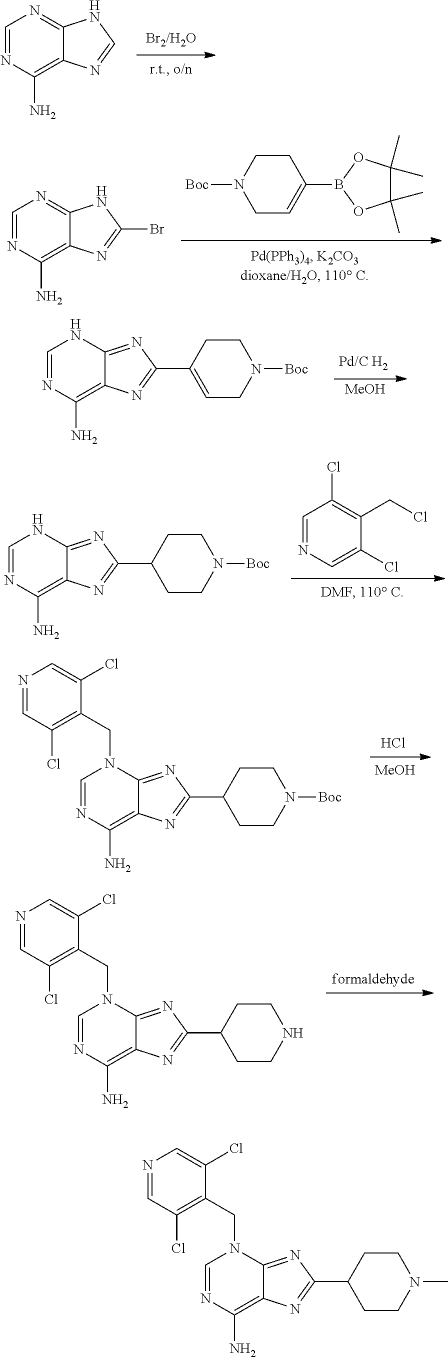

### Step 1

To a solution of 9H-purin-6-amine (2.2 g, 0.016 mol) in H₂O (200 mL), was added Br₂ (6 mL). The reaction was stirred at r.t. overnight. The reaction mixture was concentrated in vacuo. The residue was washed with water (15 mL×2) and EA (15 mL×2) to give 8-bromo-9H-purin-6-amine (1.8 g, yield: 52.9%) as a yellow solid.

### Step 2

To a solution of 8-bromo-9H-purin-6-amine (1 g, 4.6 mmol) in dioxane (5 mL) and H₂O (1 mL) was added tert-butyl 4-(4,4,5,5-tetramethyl-1,3,2-dioxaborolan-2-yl)-5,6-dihydropyridine-1(2H)-carboxylate (1.7 g, 5.6 mol), Pd (PPh₃)₄ (0.52 g, 0.46 mmol) and K₂CO₃ (1.2 g, 9.2 mmol). The reaction was stirred at 110° C. overnight. After cooling down to room temperature, the reaction mixture was concentrated and purified by silica gel column (PE/EA=1/1˜EA) to give tert-butyl 4-(6-amino-3H-purin-8-yl)-5,6-dihydropyridine-1(2H)-carboxylate (420 mg, yield: 28%) as a white solid. MS: m/z 317.1 (M+H)⁺.

### Step 3

To a solution of tert-butyl 4-(6-amino-3H-purin-8-yl)-5,6-dihydropyridine-1(2H)-carboxylate (420 mg, 1.32 mmol) in MeOH (8 mL) was added Pd/C (40 mg). The reaction was stirred at 80° C. overnight under H₂ atmosphere. After cooling down to room temperature, filtered and the filtrate was concentrated to give tert-butyl 4-(6-amino-3H-purin-8-yl)piperidine-1-carboxylate (280 mg, yield: 66.3%) as a white solid. MS: m/z 319.2 (M+H)⁺.

### Step 4

To a solution of tert-butyl 4-(6-amino-3H-purin-8-yl)piperidine-1-carboxylate (260 mg, 0.81 mmol) in DMF (5 mL) was added 3,5-dichloro-4-(chloromethyl)pyridine (354 mg, 1.78 mmol). The reaction was stirred at 110° C. overnight. After cooling down to room temperature, the reaction mixture was concentrated and purified by silica gel column (PE/EA=1/1) to give tert-butyl 4-(6-amino-3-((3,5-dichloropyridin-4-yl)methyl)-3H-purin-8-yl)piperidine-1-carboxylate (280 mg, yield: 71.7%) as a white solid. MS: m/z 478.1 (M+H)⁺.

### Step 5

To a solution of tert-butyl 4-(6-amino-3-((3,5-dichloropyridin-4-yl)methyl)-3H-purin-8-yl)piperidine-1-carboxylate (260 mg, 0.54 mmol) in HCl/MeOH (5 mL). The reaction was stirred at r.t. for 1 h. Then the reaction mixture was concentrated and purified by prep-HPLC to give 3-((3,5-dichloropyridin-4-yl)methyl)-8-(piperidin-4-yl)-3H-purin-6-amine (140 mg, yield: 68.2%) as a white solid. MS: m/z 377.8 (M+H)⁺.

¹H NMR (400 MHz, DMSO-d₆): δ=8.65 (s, 2H), 8.26 (s, 1H), 7.73 (brs, 2H), 5.70 (s, 2H), 2.96-2.93 (m, 2H), 2.70-2.66 (m, 1H), 2.59-2.56 (m, 2H), 1.84-1.78 (m, 2H), 1.62-1.54 (m, 2H). MS: m/z 377.8 (M+H)⁺.

### Step 6

To a solution of 3-((3,5-dichloropyridin-4-yl)methyl)-8-(piperidin-4-yl)-3H-purin-6-amine (120 mg, 0.25 mmol) in MeOH (5 mL) was added formaldehyde (8 mg, 0.27 mmol) and NaBH₃CN (12 mg, 0.3 mmol). The reaction mixture was stirred at r.t. overnight. Then the reaction mixture was concentrated and purified by prep-HPLC to give 3-((3,5-dichloropyridin-4-yl)methyl)-8-(1-methylpiperidin-4-yl)-3H-purin-6-amine (27 mg, yield: 21%) as a white solid.

¹H NMR (400 MHz, DMSO-d₆): δ=8.64 (s, 2H), 8.30 (s, 1H), 7.73 (brs, 2H), 5.70 (s, 2H), 2.76-2.73 (m, 2H), 2.56-2.53 (m, 1H), 2.17 (s, 3H), 1.98-1.97 (m, 2H), 1.84-1.80 (m, 2H), 1.74-1.68 (m, 2H). MS: m/z 391.8 (M+H)⁺.

### Example 4: Synthesis of 3-((3,5-dichloropyridin-4-yl)methyl)-8-(3-(trifluoromethyl)piperidin-4-yl)-3H-purin-6-amine

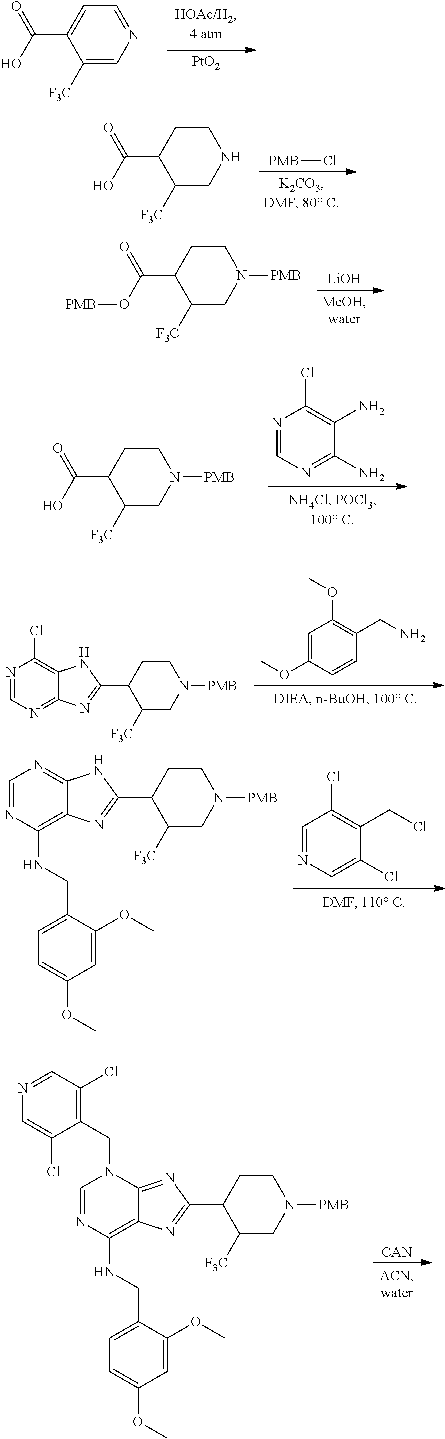

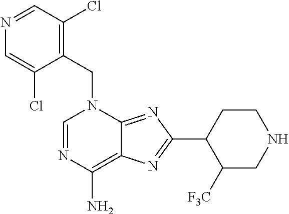 [SMILES: Nc1ncn(Cc2c(Cl)cncc2Cl)c2nc(C3CCNCC3C(F)(F)F)nc1-2]

### Step 1

A solution of 3-(trifluoromethyl)isonicotinic acid (2 g, 10.47 mmol) and PtO₂ (1 g, 50% wt.) in HOAc (20 mL) was stirred at room temperature under H₂ (4 atm) overnight. Then the reaction mixture was concentrated under reduce pressure to give 3-(trifluoromethyl)piperidine-4-carboxylic acid (1.9 g, yield: 95%) as a white solid.

### Step 2

A solution of 3-(trifluoromethyl)piperidine-4-carboxylic acid (430 mg, 2.18 mmol), PMBCl (1.36 g, 8.73 mmol) and K₂CO₃ (903 mg, 6.55 mmol) in DMF (15 mL) was stirred at 80° C. overnight. After cooling down to room temperature, the reaction mixture was concentrated to give residue which was purification by silica gel column (PE/EA=5/1) to give 4-methoxybenzyl 1-(4-methoxybenzyl)-3-(trifluoromethyl)piperidine-4-carboxylate (470 mg, yield: 49%) as yellow oil. MS: m/z 438.3 (M+H)⁺.

### Step 3

To a solution of 4-methoxybenzyl 1-(4-methoxybenzyl)-3-(trifluoromethyl)piperidine-4-carboxylate (800 mg, 1.8 mmol) in MeOH (15 mL) and water (5 mL) was added LiOH (86 mg, 3.6 mmol). The reaction mixture was stirred at 60° C. for 6 hrs. After cooling down to room temperature, MeOH was removed under reduced pressure. The remaining aqueous solution was neutralized with 1N HCl and extracted with i-propanol/CH₃Cl (⅓, 100 mL×3). The extracts were dried over anhydrous Na₂SO₄, filtered and concentrated to give 1-(4-methoxybenzyl)-3-(trifluoromethyl)piperidine-4-carboxylic acid (640 mg, yield: 99%) as yellow oil. MS: m/z 318.3 (M+H)⁺.

### Step 4

To a solution of 1-(4-methoxybenzyl)-3-(trifluoromethyl)piperidine-4-carboxylic acid (590 mg, 1.86 mmol), 6-chloropyrimidine-4,5-diamine (223 mg, 1.55 mmol) and NH₄Cl (246 mg, 4.65 mmol) in POCl₃ (15 mmol) was stirred at 100° C. overnight. After cooling down to room temperature, the POCl₃ was removed under reduce pressure. The residue was neutralized with NH₃.H₂O and purified by silica gel column (DCM/MeOH=10/1) to give 6-chloro-8-(1-(4-methoxybenzyl)-3-(trifluoromethyl)piperidin-4-yl)-7H-purine (300 mg, yield: 45%) as a yellow solid.

¹H NMR (400 MHz, DMSO-d₆): δ 13.85 (s, 1H), 8.70 (s, 1H), 7.26 (d, J=7.6 Hz, 2H), 6.92 (d, J=8.8 Hz, 2H), 3.75 (s, 3H), 3.59-3.51 (m, 2H), 3.16-3.14 (m, 3H), 2.90 (d, J=11.2 Hz, 1H), 2.13-2.11 (m, 2H), 2.11-1.97 (m, 2H). MS: m/z 426.2 (M+H)⁺.

### Step 5

To a solution of 6-chloro-8-(1-(4-methoxybenzyl)-3-(trifluoromethyl)piperidin-4-yl)-7H-purine (250 mg, 0.588 mmol), DMBNH₂ (118 mg, 0.71 mmol) and DIEA (183 mg, 1.42 mmol) in n-BuOH (5 mL) was stirred at 100° C. overnight. After cooling down to room temperature, the reaction mixture was concentrated to give a residue which was purified by silica gel column (DCM/MeOH=10/1) to give N-(2,4-dimethoxybenzyl)-8-(1-(4-methoxybenzyl)-3-(trifluoromethyl)piperidin-4-yl)-9H-purin-6-amine (300 mg, yield: 91%) as a yellow solid. MS: m/z 557.3 (M+H)⁺.

### Step 6

To a solution of N-(2,4-dimethoxybenzyl)-8-(1-(4-methoxybenzyl)-3-(trifluoromethyl)piperidin-4-yl)-9H-purin-6-amine (270 mg, 0.49 mmol) and 3,5-dichloro-4-(chloromethyl)pyridine (95 mg, 0.49 mmol) in DMF (8 mL) was stirred at 110° C. overnight. After cooling down to room temperature, the reaction mixture was concentrated to give a residue which was purified by prep-TLC (DCM/MeOH=20/1) to give 3-((3,5-dichloropyridin-4-yl)methyl)-N-(2,4-dimethoxybenzyl)-8-(1-(4-methoxybenzyl)-3-(trifluoromethyl)piperidin-4-yl)-3H-purin-6-amine (150 mg, yield: 42.9%) as a yellow solid.

### Step 7

To a solution of 3-((3,5-dichloropyridin-4-yl)methyl)-N-(2,4-dimethoxybenzyl)-8-(1-(4-methoxybenzyl)-3-(trifluoromethyl)piperidin-4-yl)-3H-purin-6-amine (140 mg, 0.196 mmol) in ACN (2 mL) and water (2 mL) was added CAN (537 mg, 0.98 mmol). After stirring at room temperature overnight, the reaction mixture was concentrated to give a residue which was purified by prep-HPLC (05-95, NH₄HCO₃) to give 3-((3,5-dichloropyridin-4-yl)methyl)-8-(3-(trifluoromethyl)piperidin-4-yl)-3H-purin-6-amine (1.4 mg, 1.6%) as a white solid.

¹H NMR (400 MHz, CDCl₃): δ=8.61 (s, 2H), 7.82 (s, 1H), 5.82 (s, 2H), 3.45-3.28 (m, 1H), 3.27-3.12 (m, 1H), 2.91-2.89 (m, 1H), 2.89-2.71 (m, 4H), 2.23-2.11 (m, 2H). MS: m/z 446.1 (M+H)⁺.

### Example 5: Synthesis of 3-((3,5-dichloropyridin-4-yl)methyl)-8-((3R,4S)-3-methylpiperidin-4-yl)-3H-purin-6-amine

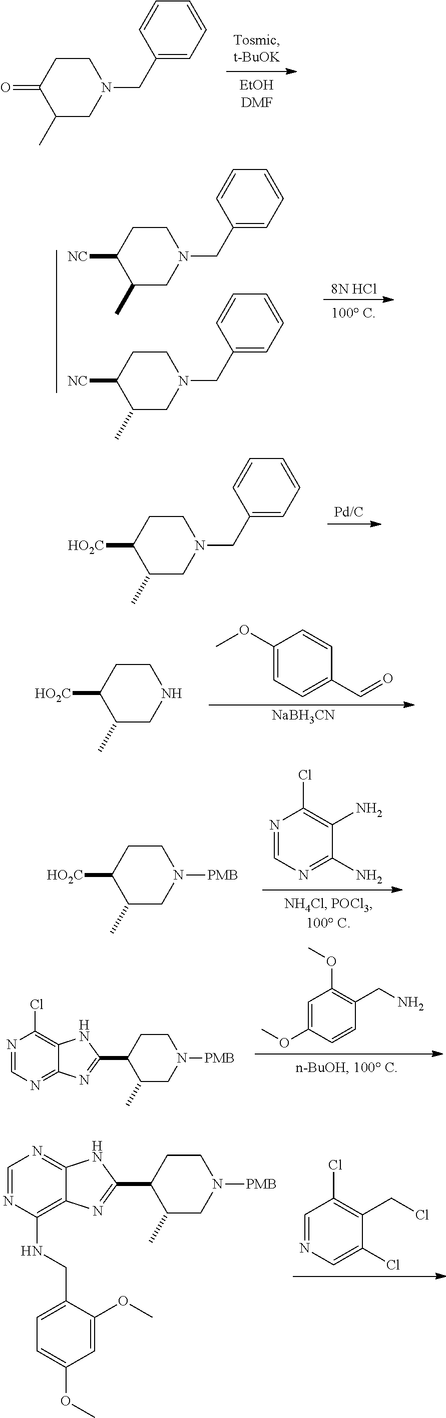

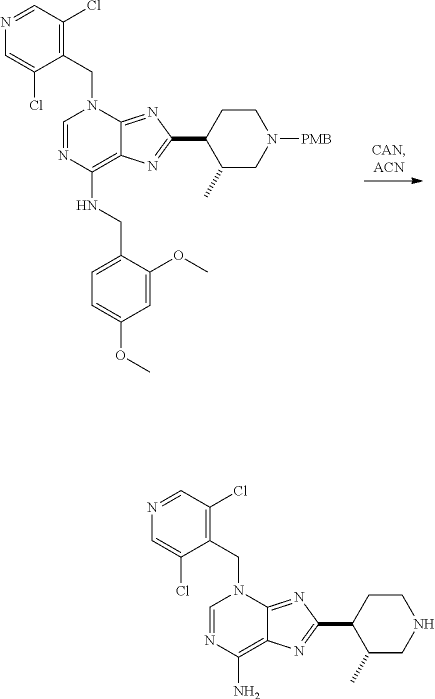

### Step 1

To a solution of 1-benzyl-3-methylpiperidin-4-one (10.0 g, 49.1 mmol) in DMF (100 mL) was added t-BuOK (16.5 g, 147.0 mmol) under 0° C. The solution was stirred at 0° C. for further 15 min. Then to the reaction mixture was added tosmic (14.4 g, 73.7 mmol) and EtOH (6.6 mL). The solution mixture was stirred at 50° C. for 2 hrs. The reaction mixture was concentrated under reduce pressure and purified by flash (EA in PE=17%) to give (3R,4S)-1-benzyl-3-methylpiperidine-4-carbonitrile (4.88 g, yield: 46%) and (3S,4S)-1-benzyl-3-methylpiperidine-4-carbonitrile (3.66 g, yield: 35%) as a yellow solid.

¹H NMR (400 MHz, CDCl₃): δ=7.34-7.24 (m, 5H), 3.48 (s, 2H), 2.90-2.85 (m, 2H), 2.08-1.85 (m, 5H), 1.67-1.59 (m, 1H), 1.07 (d, J=6.4 Hz, 3H). MS: m/z 215.3 (M+H)⁺.

### Step 2

A solution of (3R,4S)-1-benzyl-3-methylpiperidine-4-carbonitrile (3 g, 14.0 mmol) in HCl (8 N) (30 mL) was stirred at 100° C. overnight. The solvent was removed under reduce pressure. The residue was purified by flash chromatography on reverse phase silica gel (ACN/H₂O=8%) to give (3R,4S)-1-benzyl-3-methylpiperidine-4-carboxylic acid (3.042 g, yield: 93.3%) as a white solid. MS: m/z 234.3 (M+H)⁺.

### Step 3

To a solution of (3R,4S)-1-benzyl-3-methylpiperidine-4-carboxylic acid (3 g, 12.8 mmol) in MeOH (40 mL) was added Pd/C (1.5 g). The reaction mixture was stirred at room temperature under H₂ overnight. The solution was filtered. The filtrate was concentrated under reduce pressure to give (3R,4S)-3-methylpiperidine-4-carboxylic acid (1.627 g, yield: 88%) as a white solid.

¹H NMR (400 MHz, DMSO-d₆): δ=9.23 (s, 2H), 3.36-3.22 (m, 1H), 3.18-3.14 (m, 1H), 2.85-2.78 (m, 1H), 2.61-2.55 (m, 1H), 2.22-2.15 (m, 1H), 2.01-1.92 (m, 2H), 1.80-1.69 (m, 1H), 0.87 (d, J=6.8 Hz, 3H).

### Step 4

To a solution of (3R,4S)-3-methylpiperidine-4-carboxylic acid (1.6 g, 11.0 mmol) in MeOH (16 mL) was added 4-methoxybenzaldehyde (3 g, 22.0 mmol). The reaction mixture was stirred at 30° C. for 2 hrs. Then to the reaction mixture was added NaBH₃CN (2.1 g, 33.0 mmol). The mixture was stirred at 30° C. overnight. The reaction mixture was concentrated. The residue was purified by flash chromatography on reverse phase silica gel (H₂O) to give (3R,4S)-1-(4-methoxybenzyl)-3-methylpiperidine-4-carboxylic acid (1.4 g, yield: 50%) as yellow oil.

### Step 5

A solution of (3R,4S)-1-(4-methoxybenzyl)-3-methylpiperidine-4-carboxylic acid (845 mg, 3.2 mmol), 6-chloropyrimidine-4,5-diamine (462 mg, 3.2 mmol) and NH₄Cl (510 mg, 9.6 mmol) in POCl₃ (8 ml) was stirred at 100° C. overnight. The solution was removed under reduce pressure. The residue was neutralized with NH₃.H₂O and purified by silica gel column (DCM/MeOH=10/1) to give 6-chloro-8-((3R,4S)-1-(4-methoxybenzyl)-3-methylpiperidin-4-yl)-7H-purine (528 mg, yield: 54.2%) as a white solid. MS: m/z 372.3 (M+H)⁺.

### Step 6

A solution of 6-chloro-8-((3R,4S)-1-(4-methoxybenzyl)-3-methylpiperidin-4-yl)-7H-purine (300 mg, 0.8 mmol), DMBNH₂ (148.5 mg, 0.8 mmol) and DIEA (156.4 mg, 1.2 mmol) in n-BuOH (3 mL) was stirred at 100° C. overnight. The solution was removed under reduce pressure. The residue was purified by silica gel column (DCM/MeOH=10/1) to give N-(2,4-dimethoxybenzyl)-8-((3R,4S)-1-(4-methoxybenzyl)-3-methylpiperidin-4-yl)-9H-purin-6-amine (342 mg, yield: 84%) as a yellow solid. MS: m/z 503.4 (M+H)⁺.

### Step 7

A solution of N-(2,4-dimethoxybenzyl)-8-((3R,4S)-1-(4-methoxybenzyl)-3-methylpiperidin-4-yl)-9H-purin-6-amine (250 mg, 0.5 mmol) and 3,5-dichloro-4-(chloromethyl)pyridine (200 mg, 0.5 mmol) in DMF (3 mL) was stirred at 110° C. overnight. The solution was removed under reduce pressure. The residue was purified by prep-TLC (DCM/MeOH=20/1) to give 3-((3,5-dichloropyridin-4-yl)methyl)-N-(2,4-dimethoxybenzyl)-8-((3R,4S)-1-(4-methoxybenzyl)-3-methylpiperidin-4-yl)-3H-purin-6-amine (180 mg, yield: 54.7%) as a yellow solid. MS: m/z 662.4 (M+H)⁺.

### Step 8

To a solution of 3-((3,5-dichloropyridin-4-yl)methyl)-N-(2,4-dimethoxybenzyl)-8-((3R,4S)-1-(4-methoxybenzyl)-3-methylpiperidin-4-yl)-3H-purin-6-amine (160 mg, 0.2 mmol) in ACN (2 mL) and water (2 mL) was added CAN (663 mg, 1.2 mmol). The reaction was stirred at room temperature overnight. The solution was removed under reduce pressure. The residue was purified by prep-HPLC (05-95, NH₄HCO₃) to give 3-((3,5-dichloropyridin-4-yl)methyl)-8-((3R,4S)-3-methylpiperidin-4-yl)-3H-purin-6-amine (4 mg, 1.6%) as a yellow solid.

¹H NMR (400 MHz, CDCl₃): δ=8.61 (s, 2H), 7.69 (s, 1H), 5.83 (s, 2H), 3.21-3.13 (m, 2H), 2.77-2.63 (m, 2H), 2.41 (t, J=12.0 Hz, 1H), 2.07-1.72 (m, 3H), 0.72 (d, J=6.8 Hz, 3H). MS: m/z 392.1 (M+H)⁺.

The following compound was prepared using analogous procedures to Example 5.

| Name | Structure | Characterization |
|---|---|---|
| 3-((3,5-dichloropyridin- 4-yl)methyl)-8-((3S,4S)- 3-methylpiperidin-4-yl)- 3H-purin-6-amine | 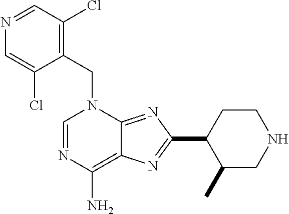 | ¹H NMR (400 MHz, CD₃OD): δ = 8.60 (s, 1H), 8.50 (s, 2H), 5.84 (s, 2H), 3.37-3.31 (m, 2H), 3.03-3.02 (m, 2H), 2.90-2.89 (m, 1H), 2.78- 2.75 (m, 1H), 2.02-1.91 (m, 2H), 0.68 (d, J = 6.4 Hz, 3H). MS: m/z 392.1 (M + H⁺). |

The following compounds were prepared using various analogous procedures to Examples 1 to 5.

| Name | Structure | Characterization |
|---|---|---|
| 3-[(3,5-dichloro(4- pyridyl))methyl]-8-(1,3- dimethylpyrazol-5-yl)-3- hydropurine-6-ylamine | 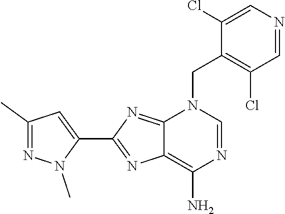 | MS: m/z 407.2 (M + H⁺). |
| 8-benzimidazol-2-yl-3-[(3,5- dichloro(4-pyridyl))methyl]-3- hydropurine-6-ylamine | 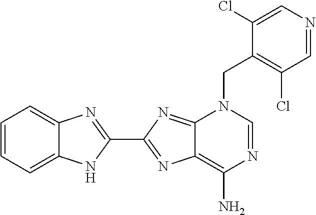 [SMILES: Nc1ncn(Cc2c(Cl)cncc2Cl)c2nc(-c3nc4ccccc4[nH]3)nc1-2] | MS: m/z 412.2 (M + H⁺). |
| 3-[(3,5-dichloro(4- pyridyl))methyl]-8-(2,5- difluorophenyl)-3-hydropurine-6- ylamine | 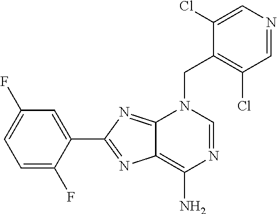 [SMILES: Nc1ncn(Cc2c(Cl)cncc2Cl)c2nc(-c3cc(F)ccc3F)nc1-2] | MS: m/z 408.2 (M + H⁺). |
| 3-[(3,5-dichloro(4- pyridyl))methyl]-8-[4- (trifluoromethyl)(3-pyridyl)]-3- hydropurine-6-ylamine | 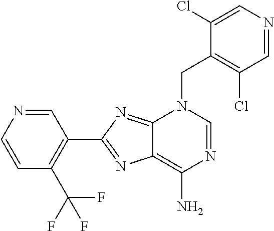 [SMILES: Nc1ncn(Cc2c(Cl)cncc2Cl)c2nc(-c3cnccc3C(F)(F)F)nc1-2] | MS: m/z 441.2 (M + H⁺). |
| 3-[(2,4-dichloro(3- pyridyl))methyl]-8-phenyl-3- hydropurine-6-ylamine | 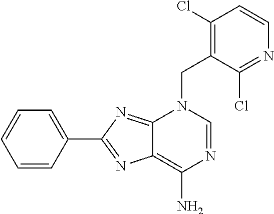 [SMILES: Nc1ncn(Cc2c(Cl)ccnc2Cl)c2nc(-c3ccccc3)nc1-2] | MS: m/z 372.2 (M + H⁺). |
| ({6-amino-3-[(3,5-dichloro(4- pyridyl))methyl](3-hydropurin-8- yl)}methyl)diethylamine | 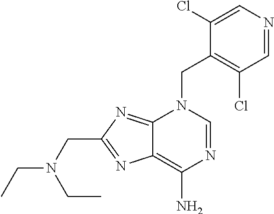 [SMILES: CCN(CC)Cc1nc2c(N)ncn(Cc3c(Cl)cncc3Cl)c-2n1] | MS: m/z 381.3 (M + H⁺). |
| 8-(4-amino(3-pyridyl))-3-[(3,5- dichloro(4-pyridyl))methyl]-3- hydropurine-6-ylamine | 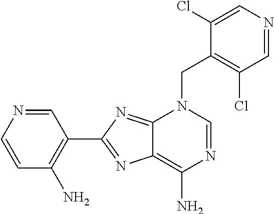 [SMILES: Nc1ccncc1-c1nc2c(N)ncn(Cc3c(Cl)cncc3Cl)c-2n1] | MS: m/z 388.2 (M + H⁺). |
| 3-[(3,5-dichloro(4- pyridyl))methyl]-8-(2-5,6,7,8- tetrahydronaphthyl)-3-hydropurine- 6-ylamine | 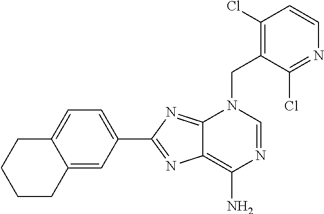 [SMILES: Nc1ncn(Cc2c(Cl)ccnc2Cl)c2nc(-c3ccc4c(c3)CCCC4)nc1-2] | MS: m/z 426.3 (M + H⁺). |
| 8-(2H-benzo[d]1,3-dioxolen-5-yl)- 3-[(3,5-dichloro(4- pyridyl))methyl]-3-hydropurine-6- ylamine | 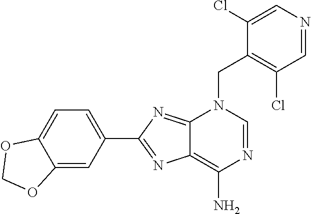 [SMILES: Nc1ncn(Cc2c(Cl)cncc2Cl)c2nc(-c3ccc4c(c3)OCO4)nc1-2] | MS: m/z 416.3 (M + H⁺). |
| 8-benzothiazol-6-yl-3-[(3,5- dichloro(4-pyridyl))methyl]-3- hydropurine-6-ylamine | 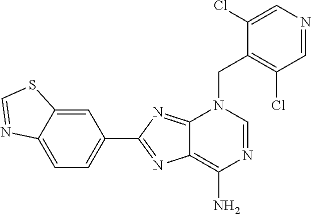 [SMILES: Nc1ncn(Cc2c(Cl)cncc2Cl)c2nc(-c3ccc4ncsc4c3)nc1-2] | MS: m/z 429.3 (M + H⁺). |
| 3-[(3,5-dichloro(4- pyridyl))methyl]-8-[(4- fluorophenyl)methyl]-3- hydropurine-6-ylamine | 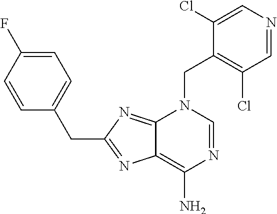 [SMILES: Nc1ncn(Cc2c(Cl)cncc2Cl)c2nc(Cc3ccc(F)cc3)nc1-2] | MS: m/z 404.3 (M + H⁺). |
| 8-adamantan-2-yl-3-[(3,5- dichloro(4-pyridyl))methyl]-3- hydropurine-6-ylamine | 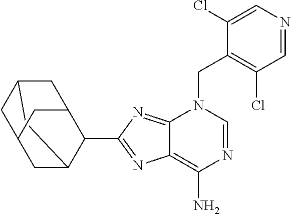 [SMILES: Nc1ncn(Cc2c(Cl)cncc2Cl)c2nc(C3C4CC5CC(C4)CC3C5)nc1-2] | MS: m/z 430.3 (M + H⁺). |
| 3-[(2,4-dichloro(3- pyridyl))methyl]-8-(1,3- dimethylpyrazol-5-yl)-3- hydropurine-6-ylamine | 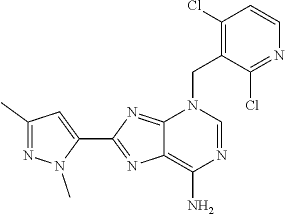 [SMILES: Cc1cc(-c2nc3c(N)ncn(Cc4c(Cl)ccnc4Cl)c-3n2)n(C)n1] | MS: m/z 390.2 (M + H⁺). |
| 3-[(2,4-dichloro(3- pyridyl))methyl]-8-(2,5- difluorophenyl)-3-hydropurine-6- ylamine | 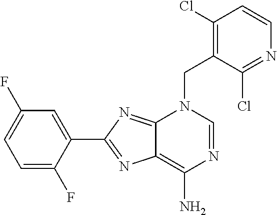 [SMILES: Nc1ncn(Cc2c(Cl)ccnc2Cl)c2nc(-c3cc(F)ccc3F)nc1-2] | MS: m/z 408.2 (M + H⁺). |
| 8-adamantan-2-yl-3-[(2,4- dichioro(3-pyridyl))methyl]-3- hydropurine-6-ylamine | 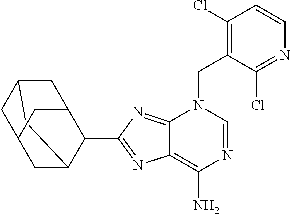 [SMILES: Nc1ncn(Cc2c(Cl)ccnc2Cl)c2nc(C3C4CC5CC(C4)CC3C5)nc1-2] | MS: m/z 430.3 (M + H⁺). |
| 3-[(2,4-dichloro(3- pyridyl))methyl]-8-(2-5,6,7,8- tetrahydronaphthyl)-3-hydropurine- 6-ylamine | 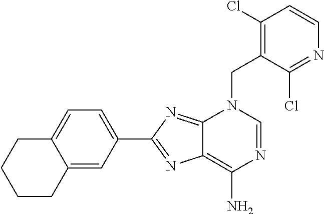 [SMILES: Nc1ncn(Cc2c(Cl)ccnc2Cl)c2nc(-c3ccc4c(c3)CCCC4)nc1-2] | MS: m/z 426.3 (M + H⁺). |
| 3-[(2,4-dichloro(3- pyridyl))methyl]-8-[(4- fluorophenyl)methyl]-3- hydropurine-6-ylamine | 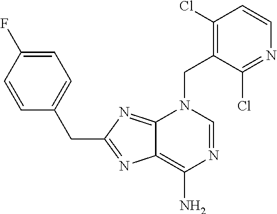 [SMILES: Nc1ncn(Cc2c(Cl)ccnc2Cl)c2nc(Cc3ccc(F)cc3)nc1-2] | MS: m/z 404.3 (M + H⁺). |
| 8-(2H-benzo[d]1,3-dioxolen-5-yl)- 3-[(2,4-dichloro(3- pyridyl))methyl]-3-hydropurine-6- ylamine | 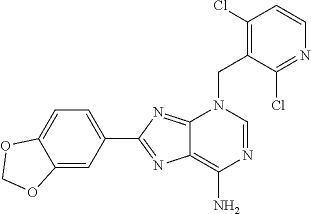 [SMILES: Nc1ncn(Cc2c(Cl)ccnc2Cl)c2nc(-c3ccc4c(c3)OCO4)nc1-2] | MS: m/z 404.3 (M + H⁺). |
| 8-benzothiazol-6-yl-3-[(2,4- dichioro(3-pyridyl))methyl]-3- hydropurine-6-ylamine | 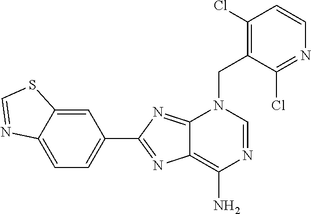 [SMILES: Nc1ncn(Cc2c(Cl)ccnc2Cl)c2nc(-c3ccc4ncsc4c3)nc1-2] | MS: m/z 429.3 (M + H⁺). |
| 8-benzo[b]furan-5-yl-3-[(3,5- dichloro(4-pyridyl))methyl]-3- hydropurine-6-ylamine | 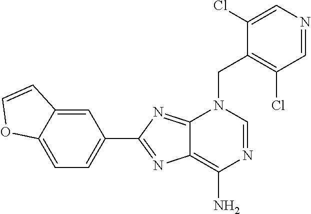 [SMILES: Nc1ncn(Cc2c(Cl)cncc2Cl)c2nc(-c3ccc4occc4c3)nc1-2] | MS: m/z 412.2 (M + H⁺). |
| 3-[(3,5-dichloro(4- pyridyl))methyl]-8-(8- hydropyrazolo[1,5-a]pyridin-3-yl)- 3-hydropurine-6-ylamine | 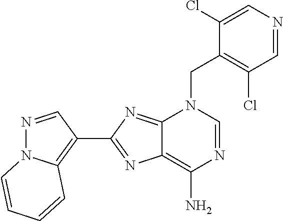 [SMILES: Nc1ncn(Cc2c(Cl)cncc2Cl)c2nc(-c3cnn4ccccc34)nc1-2] | MS: m/z 412.2 (M + H⁺). |
| 3-[(3,5-dichloro(4- pyridyl))methyl]-8-(1- methylbenzimidazol-6-yl)-3- hydropurine-6-ylamine | 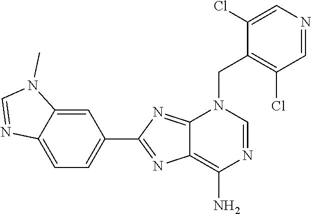 [SMILES: Cn1cnc2ccc(-c3nc4c(N)ncn(Cc5c(Cl)cncc5Cl)c-4n3)cc21] | MS: m/z 426.3 (M + H⁺). |
| 3-[(3,5-dichloro(4- pyridyl))methyl]-8-(1- methylbenzimidazol-5-yl)-3- hydropurine-6-ylamine | 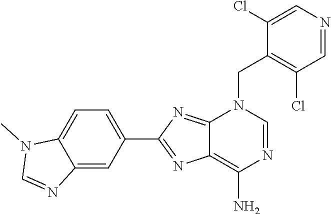 [SMILES: Cn1cnc2cc(-c3nc4c(N)ncn(Cc5c(Cl)cncc5Cl)c-4n3)ccc21] | MS: m/z 426.3 (M + H⁺). |
| 8-benzo[3,4-b]furan-6-yl-3-[(3,5- dichloro(4-pyridyl))methyl]-3- hydropurine-6-ylamine | 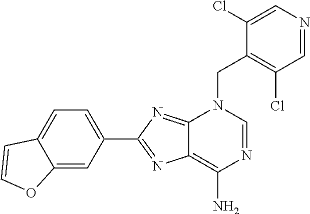 [SMILES: Nc1ncn(Cc2c(Cl)cncc2Cl)c2nc(-c3ccc4ccoc4c3)nc1-2] | MS: m/z 412.3 (M + H⁺). |
| 8-benzo[b]thiophen-5-yl-3-[(3,5- dichloro(4-pyridyl))methyl]-3- hydropurine-6-ylamine | 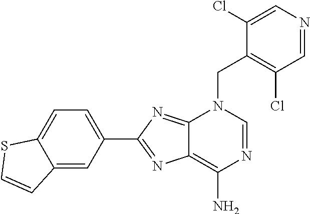 [SMILES: Nc1ncn(Cc2c(Cl)cncc2Cl)c2nc(-c3ccc4sccc4c3)nc1-2] | MS: m/z 428.3 (M + H⁺). |
| 8-benzimidazol-5-yl-3-[(3,5- dichloro(4-pyridyl))methyl]-3- hydropurine-6-ylamine | 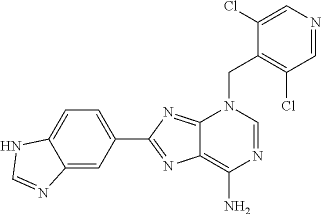 [SMILES: Nc1ncn(Cc2c(Cl)cncc2Cl)c2nc(-c3ccc4[nH]cnc4c3)nc1-2] | MS: m/z 412.2 (M + H⁺). |
| 3-[(3,5-dichloro(4- pyridyl))methyl]-8-(8- hydropyrazolo[1,5-a]pyridin-5-yl)- 3-hydropurine-6-ylamine | 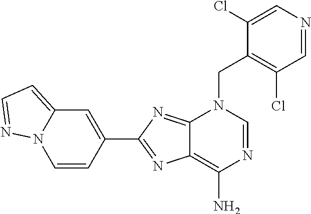 [SMILES: Nc1ncn(Cc2c(Cl)cncc2Cl)c2nc(-c3ccn4nccc4c3)nc1-2] | MS: m/z 412.3 (M + H⁺). |
| 3-[(3,5-dibromo(4- pyridyl))methyl]-8-(2- methylphenyl)-3-hydropurine-6- ylamine | 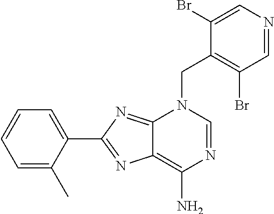 [SMILES: Cc1ccccc1-c1nc2c(N)ncn(Cc3c(Br)cncc3Br)c-2n1] | MS: m/z 475.2 (M + H⁺). |
| 3-[(4-chloro-2-fluoro(3- pyridyl))methyl]-8-(2- methylphenyl)-3-hydropurine-6- ylamine | 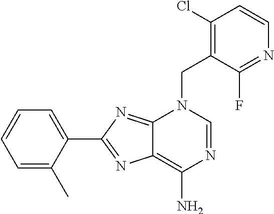 [SMILES: Cc1ccccc1-c1nc2c(N)ncn(Cc3c(Cl)ccnc3F)c-2n1] | MS: m/z 369.8 (M + H⁺). |
| 3-[(4-methyl(1,3-thiazol-5- yl))methyl]-8-(2-methylphenyl)-3- hydropurine-6-ylamine | 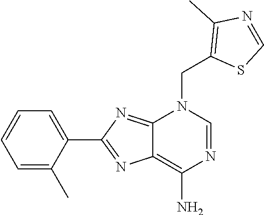 [SMILES: Cc1ccccc1-c1nc2c(N)ncn(Cc3scnc3C)c-2n1] | MS: m/z 337.4 (M + H⁺). |
| 3-[(3-chloro(2-thienyl))methyl]-8- (2-methylphenyl)-3-hydropurine-6- ylamine | 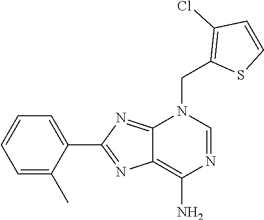 [SMILES: Cc1ccccc1-c1nc2c(N)ncn(Cc3sccc3Cl)c-2n1] | MS: m/z 356.8 (M + H⁺). |
| 3-[(3-chloro(4-pyridyl))methyl]-8- (2-methylphenyl)-3-hydropurine-6- ylamine | 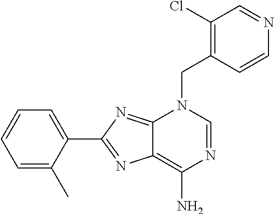 [SMILES: Cc1ccccc1-c1nc2c(N)ncn(Cc3ccncc3Cl)c-2n1] | MS: m/z 351.8 (M + H⁺). |
| 3-[(4,6-dichloropyrimidin-5- yl)methyl]-8-(2-methylphenyl)-3- hydropurine-6-ylamine | 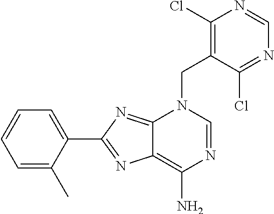 [SMILES: Cc1ccccc1-c1nc2c(N)ncn(Cc3c(Cl)ncnc3Cl)c-2n1] | MS: m/z 387.2 (M + H⁺). |
| 3-[(4-bromo-2-chloro(3- pyridyl))methyl]-8-(2- methylphenyl)-3-hydropurine-6- ylamine | 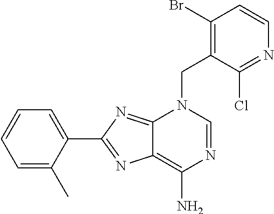 [SMILES: Cc1ccccc1-c1nc2c(N)ncn(Cc3c(Br)ccnc3Cl)c-2n1] | MS: m/z 430.7 (M + H⁺). |
| 3-[(4-bromo-1-methylpyrazol-5- yl)methyl]-8-(2-methylphenyl)-3- hydropurine-6-ylamine | 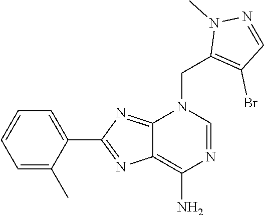 [SMILES: Cc1ccccc1-c1nc2c(N)ncn(Cc3c(Br)cnn3C)c-2n1] | MS: m/z 399.3 (M + H⁺). |
| 3-[(2,4-dichloro(3- pyridyl))methyl]-8-(2- methylphenyl)-3-hydropurine-6- ylamine | 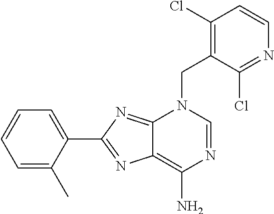 [SMILES: Cc1ccccc1-c1nc2c(N)ncn(Cc3c(Cl)ccnc3Cl)c-2n1] | MS: m/z 386.3 (M + H⁺). |
| 3-[(3,5-dibromo(4- pyridyl))methyl]-8-phenyl-3- hydropurine-6-ylamine | 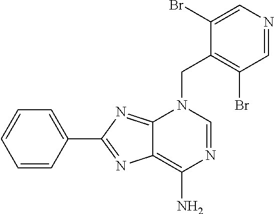 [SMILES: Nc1ncn(Cc2c(Br)cncc2Br)c2nc(-c3ccccc3)nc1-2] | MS: m/z 461.1 (M + H⁺). |
| 3-[(4-chloro-2-fluoro(3- pyridyl))methyl]-8-phenyl-3- hydropurine-6-ylamine | 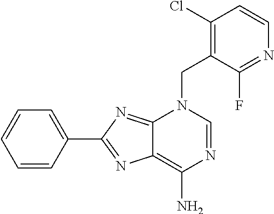 [SMILES: Nc1ncn(Cc2c(Cl)ccnc2F)c2nc(-c3ccccc3)nc1-2] | MS: m/z 354.8 (M + H⁺). |
| 3-[(4-methyl(1,3-thiazol-5- yl))methyl]-8-phenyl-3- hydropurine-6-ylamine | 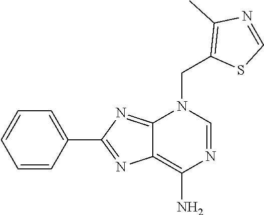 [SMILES: Cc1ncsc1Cn1cnc(N)c2nc(-c3ccccc3)nc1-2] | MS: m/z 323.4 (M + H⁺). |
| 3-[(3-chloro(2-thienyl))methyl]-8- phenyl-3-hydropurine-6-ylamine | 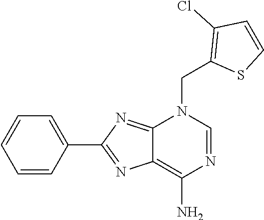 [SMILES: Nc1ncn(Cc2sccc2Cl)c2nc(-c3ccccc3)nc1-2] | MS: m/z 342.8 (M + H⁺). |
| 3-[(4,6-dichloropyrimidin-5- yl)methyl]-8-phenyl-3- hydropurine-6-ylamine | 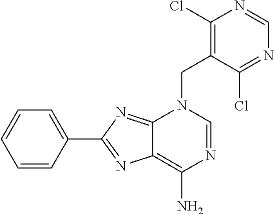 [SMILES: Nc1ncn(Cc2c(Cl)ncnc2Cl)c2nc(-c3ccccc3)nc1-2] | MS: m/z 373.2 (M + H⁺). |
| 3-[(4-bromo-2-chloro(3- pyridyl))methyl]-8-phenyl-3- hydropurine-6-ylamine | 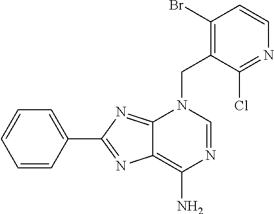 [SMILES: Nc1ncn(Cc2c(Br)ccnc2Cl)c2nc(-c3ccccc3)nc1-2] | MS: m/z 415.7 (M + H⁺). |
| 3-[(4-bromo-1-methylpyrazol-5- yl)methyl]-8-phenyl-3- hydropurine-6-ylamine | 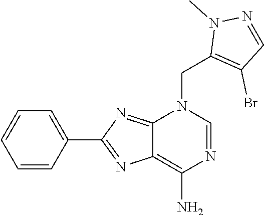 [SMILES: Cn1ncc(Br)c1Cn1cnc(N)c2nc(-c3ccccc3)nc1-2] | MS: m/z 385.2 (M + H⁺). |
| 3-[(2,4-dimethyl(1,3-thiazol-5- yl))methyl]-8-(2-methylphenyl)-3- hydropurine-6-ylamine | 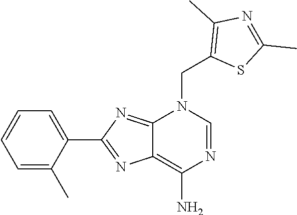 [SMILES: Cc1nc(C)c(Cn2cnc(N)c3nc(-c4ccccc4C)nc2-3)s1] | MS: m/z 351.4 (M + H⁺). |
| 3-[(3-fluoro(4-pyridyl))methyl]-8- (2-methylphenyl)-3-hydropurine-6- ylamine | 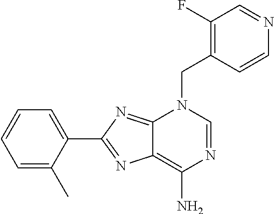 [SMILES: Cc1ccccc1-c1nc2c(N)ncn(Cc3ccncc3F)c-2n1] | MS: m/z 335.4 (M + H⁺). |
| 3-[(5-chloro(3-pyridyl))methyl]-8- (2-methylphenyl)-3-hydropurine-6- ylamine | 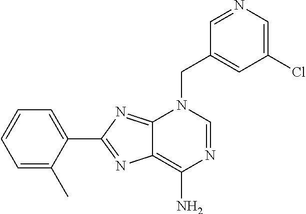 [SMILES: Cc1ccccc1-c1nc2c(N)ncn(Cc3cncc(Cl)c3)c-2n1] | MS: m/z 351.8 (M + H⁺). |
| 3-[(2-methyl(3-pyridyl))methyl]-8- (2-methylphenyl)-3-hydropurine-6- ylamine | 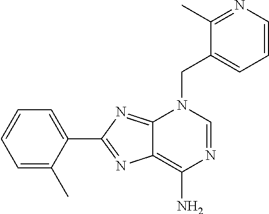 [SMILES: Cc1ccccc1-c1nc2c(N)ncn(Cc3cccnc3C)c-2n1] | MS: m/z 331.4 (M + H⁺). |
| 3-[(3,5-dibromo(4- pyridyl))methyl]-8-(8- hydropyrazolo[1,5-a]pyridin-3-yl)- 3-hydropurine-6-ylamine | 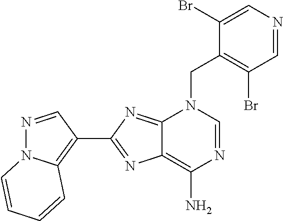 [SMILES: Nc1ncn(Cc2c(Br)cncc2Br)c2nc(-c3cnn4ccccc34)nc1-2] | MS: m/z 500.1 (M + H⁺). |
| 8-(2H-benzo[d]1,3-dioxolen-5-yl)- 3-[(3,5-dibromo(4- pyridyl))methyl]-3-hydropurine-6- ylamine | 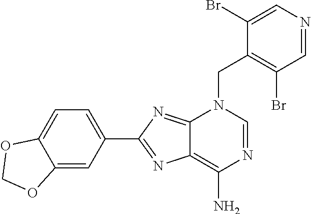 [SMILES: Nc1ncn(Cc2c(Br)cncc2Br)c2nc(-c3ccc4c(c3)OCO4)nc1-2] | MS: m/z 505.1 (M + H⁺). |
| 3-[(3,5-dibromo(4- pyridyl))methyl]-8-(2-5,6,7,8- tetrahydronaphthyl)-3-hydropurine- 6-ylamine | 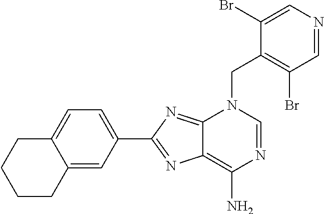 [SMILES: Nc1ncn(Cc2c(Br)cncc2Br)c2nc(-c3ccc4c(c3)CCCC4)nc1-2] | MS: m/z 515.2 (M + H⁺). |
| 3-[(3,5-dibromo(4- pyridyl))methyl]-2-methyl-8-(2- methylphenyl)-3-hydropurine-6- ylamine | 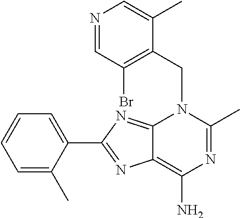 | MS: m/z 489.2 (M + H⁺). |
| 3-[(3,5-dichloro(4- pyridyl))methyl]-2-methyl-8-(2- methylphenyl)-3-hydropurine-6- ylamine | 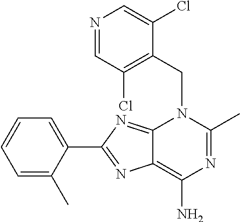 [SMILES: Cc1ccccc1-c1nc2c(N)nc(C)n(Cc3c(Cl)cncc3Cl)c-2n1] | MS: m/z 400.3 (M + H⁺). |
| 3-[(4-bromo-1-methylpyrazol-5- yl)methyl]-2-methyl-8-(2- methylphenyl)-3-hydropurine-6- ylamine | 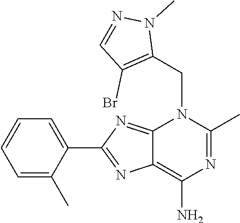 [SMILES: Cc1ccccc1-c1nc2c(N)nc(C)n(Cc3c(Br)cnn3C)c-2n1] | MS: m/z 413.3 (M + H⁺). |
| 3-[(2,4-dichloro(3- pyridyl))methyl]-2-methyl-8-(2- methylphenyl)-3-hydropurine-6- ylamine | 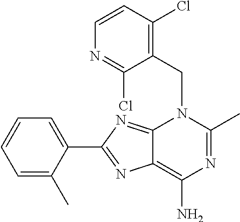 [SMILES: Cc1ccccc1-c1nc2c(N)nc(C)n(Cc3c(Cl)ccnc3Cl)c-2n1] | MS: m/z 400.3 (M + H⁺). |
| 3-[(3,5-dibromo(4- pyridyl))methyl]-8-(5,6,7,8- tetrahydronaphthyl)-3-hydropurine- 6-ylamine | 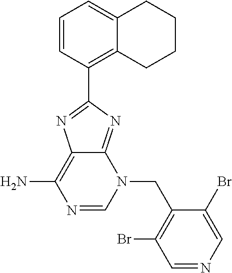 [SMILES: Nc1ncn(Cc2c(Br)cncc2Br)c2nc(-c3cccc4c3CCCC4)nc1-2] | MS: m/z 515.2 (M + H⁺). |
| 3-[(3,5-dichloro(4- pyridyl))methyl]-8-(5,6,7,8- tetrahydronaphthyl)-3-hydropurine- 6-ylamine | 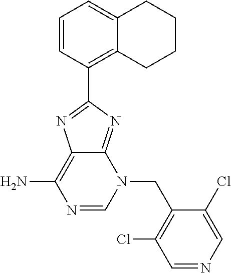 [SMILES: Nc1ncn(Cc2c(Cl)cncc2Cl)c2nc(-c3cccc4c3CCCC4)nc1-2] | MS: m/z 426.3 (M + H⁺). |
| 3-[(3,5-diiodo(4-pyridyl))methyl]- 8-(2-methylphenyl)-3-hydropurine- 6-ylamine | 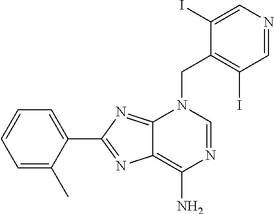 | MS: m/z 473.3 (M + H⁺). |
| 3-[(4-iodo-2-methoxy(3- pyridyl))methyl]-8-(2- methylphenyl)-3-hydropurine-6- ylamine | 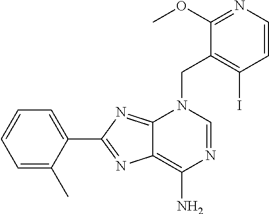 | MS: m/z 569.2 (M + H⁺). |
| 8-(2-methylphenyl)-3-[(2,3,5- trichioro(4-pyridyl))methyl]-3- hydropurine-6-ylamine | 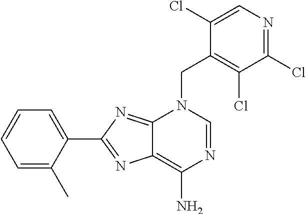 [SMILES: Cc1ccccc1-c1nc2c(N)ncn(Cc3c(Cl)cnc(Cl)c3Cl)c-2n1] | MS: m/z 420.7 (M + H⁺). |
| 3-[(4-iodo-2-methoxy(3- pyridyl))methyl]-8-phenyl-3- hydropurine-6-ylamine | 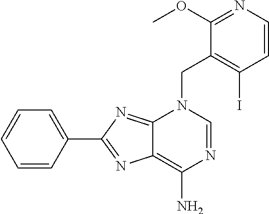 [SMILES: COc1nccc(I)c1Cn1cnc(N)c2nc(-c3ccccc3)nc1-2] | MS: m/z 459.3 (M + H⁺). |
| 8-(2,6-dimethoxyphenyl)-3-[(2,3,5- trichioro(4-pyridyl))methyl]-3- hydropurine-6-ylamine | 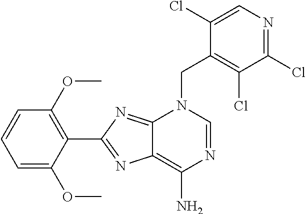 [SMILES: COc1cccc(OC)c1-c1nc2c(N)ncn(Cc3c(Cl)cnc(Cl)c3Cl)c-2n1] | MS: m/z 466.7 (M + H⁺). |
| 3-[(2,4-dimethyl(3- pyridyl))methyl]-8-(2- methylphenyl)-3-hydropurine-6- ylamine | 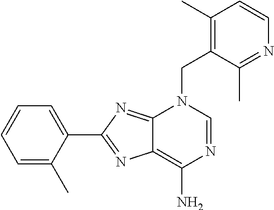 [SMILES: Cc1ccccc1-c1nc2c(N)ncn(Cc3c(C)ccnc3C)c-2n1] | MS: m/z 345.4 (M + H⁺). |
| 3-[(2,4-dimethyl(3- pyridyl))methyl]-8-phenyl-3- hydropurine-6-ylamine | 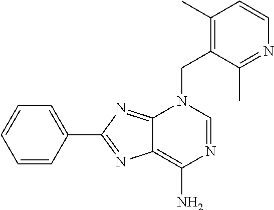 [SMILES: Cc1ccnc(C)c1Cn1cnc(N)c2nc(-c3ccccc3)nc1-2] | MS: m/z 331.4 (M + H⁺). |
| 3-[(3,5-dimethoxy(4- pyridyl))methyl]-8-(2- methylphenyl)-3-hydropurine-6- ylamine | 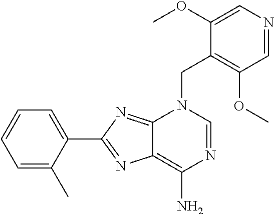 [SMILES: COc1cncc(OC)c1Cn1cnc(N)c2nc(-c3ccccc3C)nc1-2] | MS: m/z 377.4 (M + H⁺). |
| 3-[(2-amino-4,6-dichloropyrimidin- 5-yl)methyl]-8-(2-methylphenyl)-3- hydropurine-6-ylamine | 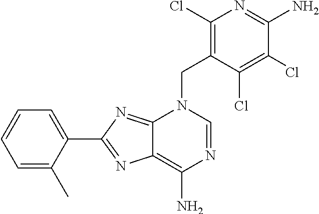 | MS: m/z 402.3 (M + H⁺). |
| 8-(2,6-dimethoxyphenyl)-3-[(2,4,5- trichioro(3-pyridyl))methyl]-3- hydropurine-6-ylamine | 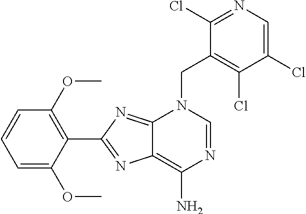 [SMILES: COc1cccc(OC)c1-c1nc2c(N)ncn(Cc3c(Cl)ncc(Cl)c3Cl)c-2n1] | MS: m/z 466.7 (M + H⁺). |
| 3-[(2,4-dimethoxy(3- pyridyl))methyl]-8-(2- methylphenyl)-3-hydropurine-6- ylamine | 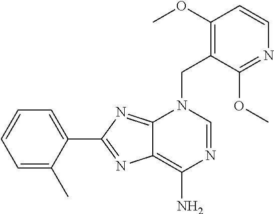 [SMILES: COc1ccnc(OC)c1Cn1cnc(N)c2nc(-c3ccccc3C)nc1-2] | MS: m/z 377.4 (M + H⁺). |

### PHARMACEUTICAL COMPOSITIONS

### Example A-1: Parenteral Pharmaceutical Composition

To prepare a parenteral pharmaceutical composition suitable for administration by injection (subcutaneous, intravenous), 1-1000 mg of a water-soluble salt of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into Size 4 capsule, or size 1 capsule (hypromellose or hard gelatin) and the capsule is closed.

### Example A-5: Topical Gel Composition

To prepare a pharmaceutical topical gel composition, a compound described herein, or a pharmaceutically acceptable salt thereof, is mixed with hydroxypropyl celluose, propylene glycol, isopropyl myristate and purified alcohol USP. The resulting gel mixture is then incorporated into containers, such as tubes, which are suitable for topical administration.

### BIOLOGICAL EXAMPLES

### Example B-1: Enzyme Assay of Inhibition of LMPTP-A

Phosphatase assays were performed in buffer containing 50 mM Bis-Tris, pH 6.0, 1 mM DTT and 0.01% Triton X-100 at 37° C. For assays conducted with 3-O-methylfluorescein phosphate (OMFP) as substrate, fluorescence was monitored continuously at λex=485 and λem=525 nm. For assays conducted with para-nitrophenylphosphate (pNPP) as substrate, the reaction was stopped by addition of 2× reaction volume of 1 M NaOH, and absorbance was measured at 405 nm. IC₅₀ values of compounds disclosed herein were determined from plots of inhibitor concentration versus percentage of enzyme activity. For inhibitor selectivity assays, each PTP was incubated with either 0.4 mM OMFP or 5 mM pNPP in the presence of 40 μM compound or DMSO. Equal units of enzyme activity, comparable to the activity of 10 nM human LMPTP-A, were used. For the inhibitor reversibility assay, 50 nM human LMPTP-A was pre-incubated with 10 μM of compound disclosed herein or DMSO for 5 min. The enzyme was diluted 100× in phosphatase assay buffer containing 0.4 mM OMFP and fluorescence was measured at the indicated time points.

Representative data for exemplary compounds disclosed herein is presented in Table 1.

TABLE 1

| Name | IC₅₀ |
|---|---|
| 3-[(3,5-dichloro(4-pyridyl))methyl]-8-(1,3-dimethylpyrazol-5-yl)-3-hydropurine-6-ylamine | A |
| 8-benzimidazol-2-yl-3-[(3,5-dichloro(4-pyridyl))methyl]-3-hydropurine-6-ylamine | A |
| 3-[(3,5-dichloro(4-pyridyl))methyl]-8-(2,5-difluorophenyl)-3-hydropurine-6-ylamine | A |
| 3-[(3,5-dichloro(4-pyridyl))methyl]-8-[4-(trifluoromethyl)(3-pyridyl)]-3-hydropurine-6-ylamine | A |
| 3-[(2,4-dichloro(3-pyridyl))methyl]-8-phenyl-3-hydropurine-6-ylamine | A |
| ({6-amino-3-[(3,5-dichloro(4-pyridyl))methyl](3-hydropurin-8-yl)}methyl)diethylamine | B |
| 8-(4-amino(3-pyridyl))-3-[(3,5-dichloro(4-pyridyl))methyl]-3-hydropurine-6-ylamine | B |
| 3-[(3,5-dichloro(4-pyridyl))methyl]-8-(2-5,6,7,8-tetrahydronaphthyl)-3-hydropurine-6-ylamine | A |
| 8-(2H-benzo[d]1,3-dioxolen-5-yl)-3-[(3,5-dichloro(4-pyridyl))methyl]-3-hydropurine-6-ylamine | A |
| 3-[(3,5-dichloro(4-pyridyl))methyl]-8-[(4-fluorophenyl)methyl]-3-hydropurine-6-ylamine | A |
| 8-adamantan-2-yl-3-[(3,5-dichloro(4-pyridyl))methyl]-3-hydropurine-6-ylamine | A |
| 3-[(2,4-dichloro(3-pyridyl))methyl]-8-(1,3-dimethylpyrazol-5-yl)-3-hydropurine-6-ylamine | A |
| 3-[(2,4-dichloro(3-pyridyl))methyl]-8-(2,5-difluorophenyl)-3-hydropurine-6-ylamine | A |
| 8-adamantan-2-yl-3-[(2,4-dichloro(3-pyridyl))methyl]-3-hydropurine-6-ylamine | A |
| 3-[(2,4-dichloro(3-pyridyl))methyl]-8-(2-5,6,7,8-tetrahydronaphthyl)-3-hydropurine-6-ylamine | A |
| 3-[(2,4-dichloro(3-pyridyl))methyl]-8-[(4-fluorophenyl)methyl]-3-hydropurine-6-ylamine | A |
| 8-benzothiazol-6-yl-3-[(2,4-dichloro(3-pyridyl))methyl]-3-hydropurine-6-ylamine | A |
| 8-benzo[b]furan-5-yl-3-[(3,5-dichloro(4-pyridyl))methyl]-3-hydropurine-6-ylamine | A |
| 3-[(3,5-dichloro(4-pyridyl))methyl]-8-(8-hydropyrazolo[1,5-a]pyridin-3-yl)-3-hydropurine-6-ylamine | A |
| 3-[(3,5-dichloro(4-pyridyl))methyl]-8-(1-methylbenzimidazol-6-yl)-3-hydropurine-6-ylamine | A |
| 3-[(3,5-dichloro(4-pyridyl))methyl]-8-(1-methylbenzimidazol-5-yl)-3-hydropurine-6-ylamine | A |
| 8-benzo[3,4-b]furan-6-yl-3-[(3,5-dichloro(4-pyridyl))methyl]-3-hydropurine-6-ylamine | A |
| 8-benzo[b]thiophen-5-yl-3-[(3,5-dichloro(4-pyridyl))methyl]-3-hydropurine-6-ylamine | A |
| 8-benzimidazol-5-yl-3-[(3,5-dichloro(4-pyridyl))methyl]-3-hydropurine-6-ylamine | A |
| 3-[(3,5-dichloro(4-pyridyl))methyl]-8-(8-hydropyrazolo[1,5-a]pyridin-5-yl)-3-hydropurine-6-ylamine | A |
| 3-[(3,5-dibromo(4-pyridyl))methyl]-8-(2-methylphenyl)-3-hydropurine-6-ylamine | A |
| 3-[(4-chloro-2-fluoro(3-pyridyl))methyl]-8-(2-methylphenyl)-3-hydropurine-6-ylamine | A |
| 3-[(4-methyl(1,3-thiazol-5-yl))methyl]-8-(2-methylphenyl)-3-hydropurine-6-ylamine | A |
| 3-[(3-chloro(2-thienyl))methyl]-8-(2-methylphenyl)-3-hydropurine-6-ylamine | A |
| 3-[(3-chloro(4-pyridyl))methyl]-8-(2-methylphenyl)-3-hydropurine-6-ylamine | A |
| 3-[(4,6-dichloropyrimidin-5-yl)methyl]-8-(2-methylphenyl)-3-hydropurine-6-ylamine | A |
| 3-[(4-bromo-2-chloro(3-pyridyl))methyl]-8-(2-methylphenyl)-3-hydropurine-6-ylamine | A |
| 3-[(4-bromo-1-methylpyrazol-5-yl)methyl]-8-(2-methylphenyl)-3-hydropurine-6-ylamine | A |
| 3-[(2,4-dichloro(3-pyridyl))methyl]-8-(2-methylphenyl)-3-hydropurine-6-ylamine | A |
| 3-[(3,5-dibromo(4-pyridyl))methyl]-8-phenyl-3-hydropurine-6-ylamine | A |
| 3-[(4-chloro-2-fluoro(3-pyridyl))methyl]-8-phenyl-3-hydropurine-6-ylamine | A |
| 3-[(4-methyl(1,3-thiazol-5-yl))methyl]-8-phenyl-3-hydropurine-6-ylamine | B |
| 3-[(3-chloro(2-thienyl))methyl]-8-phenyl-3-hydropurine-6-ylamine | A |
| 3-[(4,6-dichloropyrimidin-5-yl)methyl]-8-phenyl-3-hydropurine-6-ylamine | A |
| 3-[(4-bromo-2-chloro(3-pyridyl))methyl]-8-phenyl-3-hydropurine-6-ylamine | A |
| 3-[(4-bromo-1-methylpyrazol-5-yl)methyl]-8-phenyl-3-hydropurine-6-ylamine | A |
| 3-[(2,4-dimethyl(1,3-thiazol-5-yl))methyl]-8-(2-methylphenyl)-3-hydropurine-6-ylamine | A |
| 3-[(3-fluoro(4-pyridyl))methyl]-8-(2-methylphenyl)-3-hydropurine-6-ylamine | A |
| 3-[(5-chloro(3-pyridyl))methyl]-8-(2-methylphenyl)-3-hydropurine-6-ylamine | A |
| 3-[(2-methyl(3-pyridyl))methyl]-8-(2-methylphenyl)-3-hydropurine-6-ylamine | A |
| 3-[(3,5-dibromo(4-pyridyl))methyl]-8-(8-hydropyrazolo[1,5-a]pyridin-3-yl)-3-hydropurine-6-ylamine | A |
| 3-[(3,5-dibromo(4-pyridyl))methyl]-8-(2-5,6,7,8-tetrahydronaphthyl)-3-hydropurine-6-ylamine | A |
| 3-[(3,5-dibromo(4-pyridyl))methyl]-2-methyl-8-(2-methylphenyl)-3-hydropurine-6-ylamine | B |
| 3-[(3,5-dichloro(4-pyridyl))methyl]-2-methyl-8-(2-methylphenyl)-3-hydropurine-6-ylamine | B |
| 3-[(4-bromo-1-methylpyrazol-5-yl)methyl]-2-methyl-8-(2-methylphenyl)-3-hydropurine-6-ylamine | B |
| 3-[(2,4-dichloro(3-pyridyl))methyl]-2-methyl-8-(2-methylphenyl)-3-hydropurine-6-ylamine | B |
| 3-[(3,5-dibromo(4-pyridyl))methyl]-8-(5,6,7,8-tetrahydronaphthyl)-3-hydropurine-6-ylamine | C |
| 3-[(3,5-dichloro(4-pyridyl))methyl]-8-(5,6,7,8-tetrahydronaphthyl)-3-hydropurine-6-ylamine | C |
| 3-[(3,5-diiodo(4-pyridyl))methyl]-8-(2-methylphenyl)-3-hydropurine-6-ylamine | A |
| 3-[(4-iodo-2-methoxy(3-pyridyl))methyl]-8-(2-methylphenyl)-3-hydropurine-6-ylamine | A |
| 8-(2-methylphenyl)-3-[(2,3,5-trichloro(4-pyridyl))methyl]-3-hydropurine-6-ylamine | A |
| 3-[(4-iodo-2-methoxy(3-pyridyl))methyl]-8-phenyl-3-hydropurine-6-ylamine | A |
| 8-(2,6-dimethoxyphenyl)-3-[(2,3,5-trichloro(4-pyridyl))methyl]-3-hydropurine-6-ylamine | A |
| 3-[(2,4-dimethyl(3-pyridyl))methyl]-8-(2-methylphenyl)-3-hydropurine-6-ylamine | A |
| 3-[(2,4-dimethyl(3-pyridyl))methyl]-8-phenyl-3-hydropurine-6-ylamine | A |
| 3-[(3,5-dimethoxy(4-pyridyl))methyl]-8-(2-methylphenyl)-3-hydropurine-6-ylamine | A |
| 3-[(2-amino-4,6-dichloropyrimidin-5-yl)methyl]-8-(2-methylphenyl)-3-hydropurine-6-ylamine | A |
| 8-(2,6-dimethoxyphenyl)-3-[(2,4,5-trichloro(3-pyridyl))methyl]-3-hydropurine-6-ylamine | A |
| 3-[(2,4-dimethoxy(3-pyridyl))methyl]-8-(2-methylphenyl)-3-hydropurine-6-ylamine | A |

A: IC₅₀ is <1 μM; B: IC₅₀ is >1 μM and <20 μM; and C: IC₅₀ >20 μM.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound of Formula (I), or a pharmaceutically acceptable salt or solvate thereof, 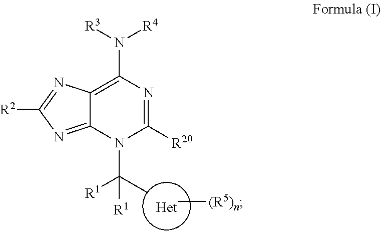
wherein:
Ring Het is heteroaryl;
each R¹ is independently hydrogen, —F, or —CH₃;
R² is halogen, —CN, —OH, —OR^{a}, —SH, —SR^{a}, C₁-C₆ alkyl, C₂-C₆ alkenyl, or C₂-C₆ alkynyl; wherein alkyl, alkenyl, and alkynyl is unsubstituted or substituted with one, two, or three R⁶;
each R⁶ is independently halogen, —CN, —OH, —OR^{a}, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —C(═O)N(R^{b})₂, —NR^{b}C(═O)R^{a};
or R² is 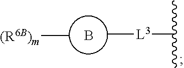
L³ is absent or C₁-C₆ alkylene;
Ring B is phenyl, heteroaryl, cycloalkyl, or heterocycloalkyl;
each R^{6a} is independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, or cycloalkyl which is unsubstituted or substituted with one, two, or three halogen, C₁-C₆ alkyl, or C₁-C₆ haloalkyl;
or two R^{6a} groups join together with the intervening atoms of ring B that connect the two R^{6a} groups to form a ring that is unsubstituted or substituted with one, two, or three halogen, —CN, —OH, C₁-C₆ alkyl, C₁-C₆ alkoxy, C₁-C₆ haloalkyl or C₁-C₆ haloalkoxy;
m is 0, 1, 2, 3, 4, or 5;
R³ is hydrogen, C₁-C₆ alkyl, C₂-C₆ alkenyl, C₂-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are independently unsubstituted or substituted with one, two, or three R¹⁰;
R⁴ is hydrogen, C₁-C₆ alkyl, C₂-C₆ alkenyl, C₂-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are independently unsubstituted or substituted with one, two, or three R¹¹;
or R⁴ is -L¹-L²-R⁷;
L¹ is —C(═O)—, —S(═O)—, —S(═O)₂—, or C₁-C₄ alkylene;
L² is absent or —CH₂—;
R⁷ is C₁-C₆ alkyl, C₂-C₆ alkenyl, C₂-C₆ alkynyl, cycloalkyl, heterocycloalkyl, aryl, monocyclic heteroaryl that contains 1-4 N atoms and 0-2 O or S atoms, monocyclic heteroaryl that contains 0-4 N atoms and 1 S atom, or bicyclic heteroaryl that contains 0-4 N atoms and 0-2 O or S atoms; wherein each alkyl, alkenyl, alkynyl, cycloalkyl, heterocycloalkyl, aryl, and heteroaryl is unsubstituted or substituted with one, two, or three R⁸;
each R⁸ is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, or cycloalkyl which is unsubstituted or substituted with one, two, or three halogen, C₁-C₆ alkyl, or C₁-C₆ haloalkyl;
or R³ and R⁴ are taken together with the nitrogen atom to which they are attached to form a heterocycloalkyl which is unsubstituted or substituted with one, two, or three R¹²;
each R⁵ is independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR⁶C(═O)R^{a}, —NR^{b}C(═O)OR^{b}, C₁-C₆ alkyl, C₁-C₆ haloalkyl, C₁-C₆ hydroxyalkyl, or cycloalkyl which is unsubstituted or substituted with one, two, or three halogen, C₁-C₆ alkyl, or C₁-C₆ haloalkyl;
n is 0, 1, 2, 3, 4, or 5;
each R¹⁰, R¹¹, and R¹² is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, or cycloalkyl which is unsubstituted or substituted with one, two, or three halogen, C₁-C₆ alkyl, or C₁-C₆ haloalkyl;
R²⁰ is hydrogen, halogen, —CN, —OH, —OR^{a}, —SH, —SR^{a}, C₁-C₆ alkyl, C₁-C₆ haloalkyl, C₁-C₆ hydroxyalkyl, cycloalkyl, or heterocycloalkyl;
each R^{a} is independently C₁-C₆ alkyl, C₂-C₆ alkenyl, C₂-C₆ alkynyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, cycloalkyl, heterocycloalkyl, aryl, and heteroaryl is independently unsubstituted or substituted with one, two, or three halogen, —OH, C₁-C₆ alkyl, or C₁-C₆ haloalkyl; and
each R^{b} is independently hydrogen, C₁-C₆ alkyl, C₂-C₆ alkenyl, C₂-C₆ alkynyl, cycloalkyl, heterocycloalkyl, aryl, or heteroaryl; wherein the alkyl, alkenyl, alkynyl, cycloalkyl, heterocycloalkyl, aryl, and heteroaryl is independently unsubstituted or substituted with one, two, or three halogen, —OH, C₁-C₆ alkyl, or C₁-C₆ haloalkyl;
or two R^{b} groups on a nitrogen atom are taken together with the nitrogen atom to which they are attached to form a heterocycloalkyl which is unsubstituted or substituted with one, two, or three halogen, C₁-C₆ alkyl, or C₁-C₆ haloalkyl;
provided that the compound is not 3-[(3,5-dichloro(4-pyridyl))methyl]-8-phenyl-3-hydropurine-6-ylamine.

2. The compound of claim 1, or a pharmaceutically acceptable salt or solvate thereof, wherein:
each R¹ is hydrogen; and
R²⁰ is hydrogen, halogen, C₁-C₆ alkyl, or C₁-C₆ haloalkyl.

3. (canceled)

4. (canceled)

5. The compound of claim 1, or a pharmaceutically acceptable salt or solvate thereof, wherein:
R³ is hydrogen;
R⁴ is hydrogen or C₁-C₆ alkyl; or
R⁴ is -L¹-L²-R⁷.

6. (canceled)

7. The compound of claim 5, or a pharmaceutically acceptable salt or solvate thereof, wherein:
L¹ is —C(═O)— or C₁-C₄ alkylene;
L² is absent or —CH₂—; and
R⁷ is aryl or monocyclic heteroaryl, each of which is optionally substituted with one, two or three R⁸.

8. (canceled)

9. The compound of claim 1, or a pharmaceutically acceptable salt or solvate thereof, wherein:
R³ and R⁴ are taken together with the nitrogen atom to which they are attached to form a heterocycloalkyl which is unsubstituted or substituted with one, two, or three R¹².

10. (canceled)

11. (canceled)

12. The compound of claim 1, or a pharmaceutically acceptable salt or solvate thereof, wherein:
Ring Het is pyridinyl, pyrazinyl, pyrimidinyl, pyridazinyl, triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl.

13.-21. (canceled)

22. The compound of claim 1, or a pharmaceutically acceptable salt or solvate thereof, wherein the compound of Formula (I) has the structure of Formula (II), or a pharmaceutically acceptable salt or solvate thereof: 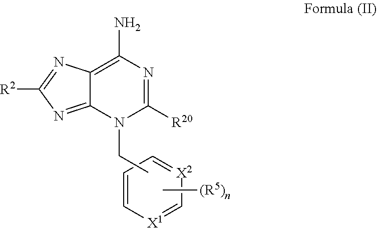
wherein:
X¹ is CH, CR⁵, or N; and
X² is CH, CR⁵, or N.

23.-30. (canceled)

31. The compound of claim 1, or a pharmaceutically acceptable salt or solvate thereof, wherein the compound of Formula (I) has the structure of Formula (V), or a pharmaceutically acceptable salt or solvate thereof: 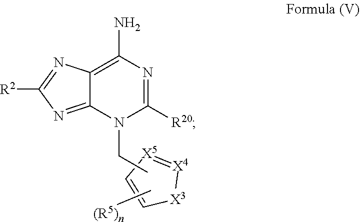
wherein,
X³ is CR⁵, CH, N, S, or O;
X⁴ is CR⁵, CH, or N; and
X⁵ is CR⁵, CH, or N.

32. (canceled)

33. (canceled)

34. (canceled)

35. (canceled)

36. The compound of claim 1, or a pharmaceutically acceptable salt or solvate thereof, wherein:
R² is 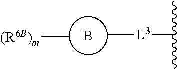
L³ is absent or C₁-C₆ alkylene;
Ring B is phenyl, heteroaryl, cycloalkyl, or heterocycloalkyl;
each R^{6a} is independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, or cycloalkyl which is unsubstituted or substituted with one, two, or three halogen, C₁-C₆ alkyl, or C₁-C₆ haloalkyl;
or two R^{6a} groups join together with the intervening atoms of ring B that connect the two R^{6a} groups to form a ring that is unsubstituted or substituted with one, two, or three halogen, —CN, —OH, C₁-C₆ alkyl, C₁-C₆ alkoxy, C₁-C₆ haloalkyl, or C₁-C₆ haloalkoxy.

37. The compound of claim 36, or a pharmaceutically acceptable salt or solvate thereof, wherein:
Ring B is phenyl, heteroaryl, or cycloalkyl.

38. (canceled)

39. The compound of claim 36, or a pharmaceutically acceptable salt or solvate thereof, wherein:
Ring B is phenyl, pyridinyl, pyrazinyl, pyrimidinyl, pyridazinyl, triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl.

40. (canceled)

41. The compound of claim 36, or a pharmaceutically acceptable salt or solvate thereof, wherein: 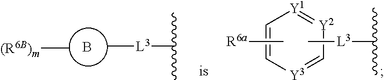
wherein:
Y¹ is CH, CR^{6a}, or N;
Y² is CH, CR^{6a}, or N; and
Y³ is CH, CR^{6a}, or N.

42. The compound of claim 1, or a pharmaceutically acceptable salt or solvate thereof, wherein the compound of Formula (I) has the structure of Formula (II-1), or a pharmaceutically acceptable salt or solvate thereof: 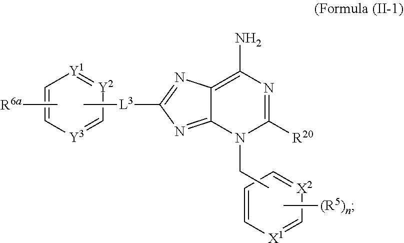
wherein:
X¹ is CH, CR⁵, or N;
X² is CH, CR⁵, or N;
Y¹ is CH, CR^{6a}, or N;
Y² is CH, CR^{6a}, or N; and
Y³ is CH, CR^{6a}, or N.

43. The compound of claim 1, or a pharmaceutically acceptable salt or solvate thereof, wherein the compound of Formula (I) has the structure of Formula (V-1), or a pharmaceutically acceptable salt or solvate thereof: 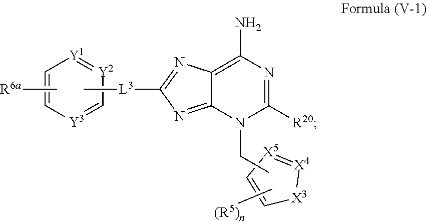
wherein:
X³ is CH, CR⁵, N, S, or O;
X⁴ is CH, CR⁵, or N;
X⁵ is CH, CR⁵, or N;
Y¹ is CH, CR^{6a}, or N;
Y² is CH, CR^{6a}, or N; and
Y³ is CH, CR^{6a}, or N.

44. The compound of claim 43, or a pharmaceutically acceptable salt or solvate thereof, wherein:
L³ is absent, or —CH₂—.

45. (canceled)

46. (canceled)

47. The compound of claim 36, or a pharmaceutically acceptable salt or solvate thereof, wherein:
two R^{6a} groups join together with the intervening atoms of ring B that connect the two R^{6a} groups to form a ring that is unsubstituted or substituted with one, two, or three halogen, —CN, —OH, C₁-C₆ alkyl, C₁-C₆ alkoxy, C₁-C₆ haloalkyl, or C₁-C₆ haloalkoxy.

48.-60. (canceled)

61. A compound that is:
3-[(3,5-dichloro(4-pyridyl))methyl]-8-(1,3-dimethylpyrazol-5-yl)-3-hydropurine-6-ylamine;
8-benzimidazol-2-yl-3-[(3,5-dichloro(4-pyridyl))methyl]-3-hydropurine-6-ylamine;
3-[(3,5-dichloro(4-pyridyl))methyl]-8-(2,5-difluorophenyl)-3-hydropurine-6-ylamine;
3-[(3,5-dichloro(4-pyridyl))methyl]-8-[4-(trifluoromethyl)(3-pyridyl)]-3-hydropurine-6-ylamine;
3-[(2,4-dichloro(3-pyridyl))methyl]-8-phenyl-3-hydropurine-6-ylamine;
({6-amino-3-[(3,5-dichloro(4-pyridyl))methyl](3-hydropurin-8-yl)}methyl)diethylamine;
8-(4-amino(3-pyridyl))-3-[(3,5-dichloro(4-pyridyl))methyl]-3-hydropurine-6-ylamine;
3-[(3,5-dichloro(4-pyridyl))methyl]-8-(2-5,6,7,8-tetrahydronaphthyl)-3-hydropurine-6-ylamine;
8-(2H-benzo[d]1,3-dioxolen-5-yl)-3-[(3,5-dichloro(4-pyridyl))methyl]-3-hydropurine-6-ylamine;
3-[(3,5-dichloro(4-pyridyl))methyl]-8-[(4-fluorophenyl)methyl]-3-hydropurine-6-ylamine;
8-adamantan-2-yl-3-[(3,5-dichloro(4-pyridyl))methyl]-3-hydropurine-6-ylamine;
3-[(2,4-dichloro(3-pyridyl))methyl]-8-(1,3-dimethylpyrazol-5-yl)-3-hydropurine-6-ylamine;
8-adamantan-2-yl-3-[(2,4-dichloro(3-pyridyl))methyl]-3-hydropurine-6-ylamine;
3-[(2,4-dichloro(3-pyridyl))methyl]-8-(2-5,6,7,8-tetrahydronaphthyl)-3-hydropurine-6-ylamine;
3-[(2,4-dichloro(3-pyridyl))methyl]-8-[(4-fluorophenyl)methyl]-3-hydropurine-6-ylamine;
8-(2H-benzo[d]1,3-dioxolen-5-yl)-3-[(2,4-dichloro(3-pyridyl))methyl]-3-hydropurine-6-ylamine;
8-benzothiazol-6-yl-3-[(2,4-dichloro(3-pyridyl))methyl]-3-hydropurine-6-ylamine;
8-benzo[b]furan-5-yl-3-[(3,5-dichloro(4-pyridyl))methyl]-3-hydropurine-6-ylamine;
3-[(3,5-dichloro(4-pyridyl))methyl]-8-(8-hydropyrazolo[1,5-a]pyridin-3-yl)-3-hydropurine-6-ylamine;
3-[(3,5-dichloro(4-pyridyl))methyl]-8-(1-methylbenzimidazol-6-yl)-3-hydropurine-6-ylamine;
3-[(3,5-dichloro(4-pyridyl))methyl]-8-(1-methylbenzimidazol-5-yl)-3-hydropurine-6-ylamine;
8-benzo[3,4-b]furan-6-yl-3-[(3,5-dichloro(4-pyridyl))methyl]-3-hydropurine-6-ylamine;
8-benzo[b]thiophen-5-yl-3-[(3,5-dichloro(4-pyridyl))methyl]-3-hydropurine-6-ylamine;
8-benzimidazol-5-yl-3-[(3,5-dichloro(4-pyridyl))methyl]-3-hydropurine-6-ylamine;
3-[(3,5-dichloro(4-pyridyl))methyl]-8-(8-hydropyrazolo[1,5-a]pyridin-5-yl)-3-hydropurine-6-ylamine;
3-[(3,5-dibromo(4-pyridyl))methyl]-8-(2-methylphenyl)-3-hydropurine-6-ylamine;
3-[(4-chloro-2-fluoro(3-pyridyl))methyl]-8-(2-methylphenyl)-3-hydropurine-6-ylamine;
3-[(4-methyl(1,3-thiazol-5-yl))methyl]-8-(2-methylphenyl)-3-hydropurine-6-ylamine;
3-[(3-chloro(2-thienyl))methyl]-8-(2-methylphenyl)-3-hydropurine-6-ylamine;
3-[(3-chloro(4-pyridyl))methyl]-8-(2-methylphenyl)-3-hydropurine-6-ylamine;
3-[(4,6-dichloropyrimidin-5-yl)methyl]-8-(2-methylphenyl)-3-hydropurine-6-ylamine;
3-[(4-bromo-2-chloro(3-pyridyl))methyl]-8-(2-methylphenyl)-3-hydropurine-6-ylamine;
3-[(4-bromo-1-methylpyrazol-5-yl)methyl]-8-(2-methylphenyl)-3-hydropurine-6-ylamine;
3-[(2,4-dichloro(3-pyridyl))methyl]-8-(2-methylphenyl)-3-hydropurine-6-ylamine;
3-[(3,5-dibromo(4-pyridyl))methyl]-8-phenyl-3-hydropurine-6-ylamine;
3-[(4-chloro-2-fluoro(3-pyridyl))methyl]-8-phenyl-3-hydropurine-6-ylamine;
3-[(4-methyl(1,3-thiazol-5-yl))methyl]-8-phenyl-3-hydropurine-6-ylamine;
3-[(3-chloro(2-thienyl))methyl]-8-phenyl-3-hydropurine-6-ylamine;
3-[(4,6-dichloropyrimidin-5-yl)methyl]-8-phenyl-3-hydropurine-6-ylamine;
3-[(4-bromo-2-chloro(3-pyridyl))methyl]-8-phenyl-3-hydropurine-6-ylamine;
3-[(4-bromo-1-methylpyrazol-5-yl)methyl]-8-phenyl-3-hydropurine-6-ylamine;
3-[(2,4-dimethyl(1,3-thiazol-5-yl))methyl]-8-(2-methylphenyl)-3-hydropurine-6-ylamine;
3-[(3-fluoro(4-pyridyl))methyl]-8-(2-methylphenyl)-3-hydropurine-6-ylamine;
3-[(5-chloro(3-pyridyl))methyl]-8-(2-methylphenyl)-3-hydropurine-6-ylamine;
3-[(2-methyl(3-pyridyl))methyl]-8-(2-methylphenyl)-3-hydropurine-6-ylamine;
3-[(3,5-dibromo(4-pyridyl))methyl]-8-(8-hydropyrazolo[1,5-a]pyridin-3-yl)-3-hydropurine-6-ylamine;
3-[(3,5-dibromo(4-pyridyl))methyl]-8-(2-5,6,7,8-tetrahydronaphthyl)-3-hydropurine-6-ylamine;
3-[(3,5-dibromo(4-pyridyl))methyl]-2-methyl-8-(2-methylphenyl)-3-hydropurine-6-ylamine;
3-[(3,5-dichloro(4-pyridyl))methyl]-2-methyl-8-(2-methylphenyl)-3-hydropurine-6-ylamine;
3-[(4-bromo-1-methylpyrazol-5-yl)methyl]-2-methyl-8-(2-methylphenyl)-3-hydropurine-6-ylamine;
3-[(2,4-dichloro(3-pyridyl))methyl]-2-methyl-8-(2-methylphenyl)-3-hydropurine-6-ylamine;
3-[(3,5-dibromo(4-pyridyl))methyl]-8-(5,6,7,8-tetrahydronaphthyl)-3-hydropurine-6-ylamine;
3-[(3,5-dichloro(4-pyridyl))methyl]-8-(5,6,7,8-tetrahydronaphthyl)-3-hydropurine-6-ylamine;
3-[(3,5-diiodo(4-pyridyl))methyl]-8-(2-methylphenyl)-3-hydropurine-6-ylamine;
3-[(4-iodo-2-methoxy(3-pyridyl))methyl]-8-(2-methylphenyl)-3-hydropurine-6-ylamine;
8-(2-methylphenyl)-3-[(2,3,5-trichloro(4-pyridyl))methyl]-3-hydropurine-6-ylamine;
3-[(4-iodo-2-methoxy(3-pyridyl))methyl]-8-phenyl-3-hydropurine-6-ylamine;
8-(2,6-dimethoxyphenyl)-3-[(2,3,5-trichloro(4-pyridyl))methyl]-3-hydropurine-6-ylamine;
3-[(2,4-dimethyl(3-pyridyl))methyl]-8-(2-methylphenyl)-3-hydropurine-6-ylamine;
3-[(2,4-dimethyl(3-pyridyl))methyl]-8-phenyl-3-hydropurine-6-ylamine;
3-[(2,4-dimethyl(3-pyridyl))methyl]-8-phenyl-3-hydropurine-6-ylamine;
3-[(3,5-dimethoxy(4-pyridyl))methyl]-8-(2-methylphenyl)-3-hydropurine-6-ylamine;
3-[(2-amino-4,6-dichloropyrimidin-5-yl)methyl]-8-(2-methylphenyl)-3-hydropurine-6-ylamine;
8-(2,6-dimethoxyphenyl)-3-[(2,4,5-trichloro(3-pyridyl))methyl]-3-hydropurine-6-ylamine; or
3-[(2,4-dimethoxy(3-pyridyl))methyl]-8-(2-methylphenyl)-3-hydropurine-6-ylamine;
or a pharmaceutically acceptable salt or solvate thereof.

62. A pharmaceutical composition comprising a compound of claim 1, or a pharmaceutically acceptable salt or solvate thereof, and a pharmaceutically acceptable excipient.

63. (canceled)

64. (canceled)

65. A method of treating a disease or condition in a mammal that would benefit by inhibition of low molecular weight protein tyrosine phosphatase (LMPTP) activity comprising administering to the mammal a compound of claim 1, or a pharmaceutically acceptable salt or solvate thereof.

66. (canceled)

67. A method of treating a metabolic disease or condition in a mammal, comprising administering to the mammal a compound of claim 1, or a pharmaceutically acceptable salt or solvate thereof.

68.-79. (canceled)
